(19) United States
(12) Patent Application Publication
(10) Pub. No.: US 2026/0228419 A1
(43) Pub. Date: Aug. 6, 2026

(54) PROVIDING ACCURATE, COMPLETE, AND CONTEXTFUL INFORMATION FROM MEDICAL NOTES WITH LLMS.

(71) Applicant: Predicta Med LTD, Tel Aviv (IL)

(72) Inventors: Dimid Duchovny, Herzliya (IL); Benjamin Getz, Herzliya (IL)

(73) Assignee: Predicta Med LTD, Tel Aviv (IL)

(21) Appl. No.: 19/409,588

(22) Filed: Dec. 4, 2025

Related U.S. Application Data
(60) Provisional application No. 63/728,116, filed on Dec. 4, 2024.

(51) Int. Cl. G06F 40/166; G06N 5/02; G16H 10/60

(52) U.S. Cl. CPC G06F 40/166; G06N 5/02; G16H 10/60

(57) ABSTRACT

The present disclosure provides a computer-implemented method for analyzing medical notes using knowledge graph enhanced language models. The method comprises receiving a medical note, processing it with a large language model to identify medical entities, determining entity types, accessing a corresponding knowledge graph containing structured medical knowledge, generating augmented context by retrieving relevant information from the knowledge graph, and reprocessing the medical note with the large language model using the augmented context to generate enhanced analysis results. Medical entities may comprise diagnoses, medications, laboratory results, or procedures. The knowledge graph may comprise ICD-10 or other standardized medical coding systems. Enhanced results may comprise medical coding verification, diagnosis accuracy checking, or consistency validation.

### CROSS-REFERENCE TO RELATED APPLICATIONS

This application claims priority from U.S. Provisional Patent No. 63/728,116 filed 4 Dec. 2024, which is hereby incorporated by reference in its entirety.

### FIELD OF INVENTION

The present disclosure relates to medical information processing systems using artificial intelligence, and more particularly to systems and methods for analyzing medical notes using knowledge graph enhanced large language models to provide accurate medical coding, diagnosis verification, and patient information summarization.

### BACKGROUND

Medical documentation and coding represent fundamental components of modern healthcare systems, where physicians generate extensive patient notes during clinical encounters to record symptoms, diagnoses, treatments, and other medical information. These notes serve multiple purposes including continuity of care, legal documentation, billing, and quality assurance. However, the complexity and volume of medical documentation present ongoing challenges for healthcare providers.

Large language models have emerged as powerful tools for natural language processing tasks across various domains, including healthcare applications. These models can process and analyze text-based medical information, extract relevant data points, and assist with various documentation tasks. However, when applied to medical contexts, general-purpose language models may lack the specialized domain knowledge and contextual understanding required for accurate medical information processing.

Medical coding systems, such as the International Classification of Diseases (ICD) and Systematized Nomenclature of Medicine Clinical Terms (SNOMED CT), provide standardized frameworks for categorizing medical conditions, procedures, and treatments. Accurate coding is fundamental for billing, research, quality metrics, and clinical decision support. The complexity of these coding systems, combined with the nuanced nature of medical language, can make accurate code assignment challenging.

Knowledge graphs have been developed in the biomedical domain to represent relationships between medical concepts, diseases, symptoms, treatments, and other healthcare entities. These structured knowledge representations can provide contextual information and domain-specific relationships that may enhance the accuracy of automated medical information processing systems.

Healthcare providers face increasing demands for documentation accuracy and completeness while managing time constraints during patient encounters. Tools that can assist physicians in reviewing, summarizing, and validating medical documentation may help address these competing demands while maintaining quality of care.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a computer-implemented method for analyzing medical notes using knowledge graph enhanced language models is provided. The method includes receiving a medical note containing medical information. The method includes processing the medical note with a large language model to identify medical entities within the medical note. The method includes determining a type of medical entity from the identified medical entities. The method includes accessing a knowledge graph corresponding to the determined type of medical entity, wherein the knowledge graph contains structured medical knowledge relevant to the type of medical entity. The method includes generating an augmented context by retrieving relevant information from the knowledge graph based on the identified medical entities. The method includes reprocessing the medical note with the large language model using the augmented context to generate enhanced medical analysis results.

According to other aspects of the present disclosure, the method may include one or more of the following features. The medical entities may include diagnoses, medications, laboratory results, or medical procedures. The knowledge graph may include ICD-10 Clinical Modification codes, SNOMED CT codes, or other standardized medical coding systems. The method may operate in a simple mode where an action for accessing the knowledge graph is predetermined based on the type of medical entity identified. The simple mode may utilize predefined query templates that are automatically populated with extracted medical entity values. The method may operate in a complex mode where an AI agent-based large language model formulates queries to access the knowledge graph as a tool. The AI agent-based large language model may utilize a ReAct framework or LangChain libraries to interact with the knowledge graph. The enhanced medical analysis results may include medical coding verification, diagnosis accuracy checking, or consistency validation. The method may include generating alerts for potential inconsistencies between the medical note content and the knowledge graph information. The method may include checking for missing laboratory test results based on diagnosis codes and their relationships in the knowledge graph.

According to another aspect of the present disclosure, a system for providing medical information analysis is provided. The system includes a processor and a memory storing instructions that, when executed by the processor, cause the system to receive medical documentation from patient medical files. The system processes the medical documentation to extract medical entities using a large language model. The system accesses biomedical knowledge graphs to obtain contextual information related to the extracted medical entities. The system generates a summary of medical information by combining the extracted medical entities with the contextual information from the knowledge graphs. The system provides the summary to assist physician preparation for patient visits.

According to other aspects of the present disclosure, the system may include one or more of the following features. The system may analyze new physician notes in real-time during patient encounters. The system may provide notifications about potential coding errors or diagnostic inconsistencies or any entity type, e.g. a CKD (chronic kidney failure) patient was prescribed a beta-lactam that is eliminated in the kidneys without dosage adjustment identified during the real-time analysis. The system may support multiple query languages for accessing different types of knowledge graphs, including SQL, SPARQL, and Cypher. The system may perform differential diagnosis analysis by querying knowledge graphs for diseases with similar symptoms or characteristics to extracted medical entities and/or anything in the patient's EHR, e.g. structured data obtained via FHIR.

According to another aspect of the present disclosure, a computer-readable medium including instructions that, when executed by a processor, cause the processor to perform a method for analyzing medical notes using knowledge graph enhanced language models is provided. The method includes receiving a medical note containing medical information. The method includes processing the medical note with a large language model to identify medical entities within the medical note. The method includes determining a type of medical entity from the identified medical entities. The method includes accessing a knowledge graph corresponding to the determined type of medical entity, wherein the knowledge graph contains structured medical knowledge relevant to the type of medical entity. The method includes generating an augmented context by retrieving relevant information from the knowledge graph based on the identified medical entities. The method includes reprocessing the medical note with the large language model using the augmented context to generate enhanced medical analysis results.

According to other aspects of the present disclosure, the computer-readable medium may include one or more of the following features. The medical entities may include diagnoses, medications, laboratory results, or medical procedures. The knowledge graph may include ICD-10 Clinical Modification codes, SNOMED CT codes, or other standardized medical coding systems. The accessing of the knowledge graph may operate in a simple mode where an action for accessing the knowledge graph is predetermined based on the type of medical entity identified. The enhanced medical analysis results may include medical coding verification, diagnosis accuracy checking, or consistency validation.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a flowchart for a method for analyzing medical notes using knowledge graph enhanced language models, according to aspects of the present disclosure.

FIG. 2 depicts a flowchart for the method of FIG. 1 with predetermined knowledge graph access, according to an embodiment.

FIG. 3 illustrates a flowchart for a method for validating medical diagnoses using cross-coding system mapping, according to aspects of the present disclosure.

FIG. 4 depicts a block diagram of a system for medical information analysis and its operational environment, according to an embodiment.

FIG. 5 illustrates an example of a flow chart related to an AI agent.

FIG. 6. Illustrates an example of an AI agent ReAct loop.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

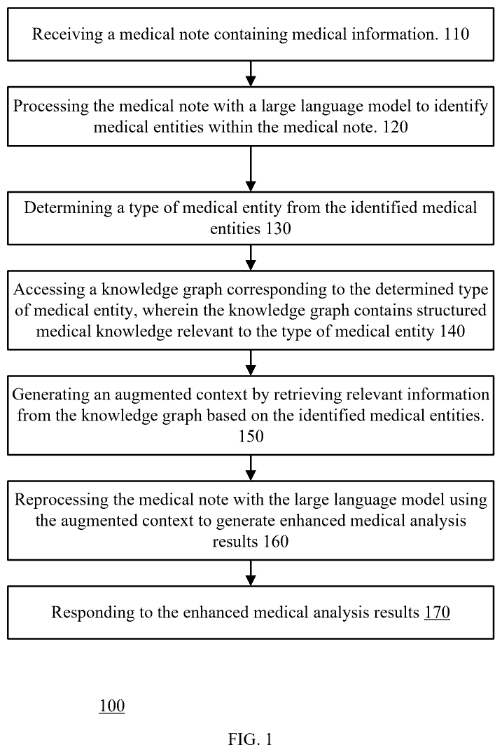

Referring to FIG. 1, a method 100 for analyzing medical notes using knowledge graph enhanced language models may provide a systematic approach for processing medical documentation through multiple stages that combine natural language processing capabilities with structured medical knowledge retrieval operations. The method 100 may coordinate interactions between language model components and knowledge graph systems to generate enhanced analytical results that demonstrate improved accuracy and clinical relevance compared to analysis performed without knowledge graph augmentation. Any reference to a knowledge graph should be applied mutatis mutandis to medical guidelines. The medical guidelines can be chunked and be use for RAG. Any reference to coding in the context of knowledge graphs should be applied mutatis mutandis to terms, concepts and identifiers.

The method 100 may begin with step 110 of receiving a medical note containing medical information. The medical note may be received through data interfaces configured to accept various medical document formats including structured electronic health record data, unstructured text documents, and standardized medical messaging formats. The receiving step may involve initial document validation, format standardization, and content preparation to ensure that medical notes are presented in formats suitable for language model analysis. The medical note may contain clinical information including patient symptoms, diagnostic assessments, treatment plans, medication prescriptions, laboratory test orders, and other healthcare-related documentation generated during patient encounters.

As shown in FIG. 1, step 110 may be followed by step 120 of processing the medical note with a large language model to identify medical entities within the medical note. The processing operation may utilize transformer-based architectures or other neural network or any other machine-learning or statistical method approaches configured to understand medical terminology, clinical abbreviations, and healthcare-specific language patterns. The language model may analyze the document content to locate diagnostic terms, medication references, laboratory test mentions, procedure descriptions, and other clinically relevant entities. Each identified entity may be classified according to predefined medical entity categories and assigned confidence scores that indicate the reliability of the extraction and classification operations. The processing may involve named entity recognition techniques that classify different portions of the medical text according to medical entity types such as diagnoses, medications, laboratory results, or medical procedures.

With continued reference to FIG. 1, step 120 may be followed by step 130 of determining a type of medical entity from the identified medical entities. The determination process may analyze the characteristics and classifications assigned during the entity identification phase to categorize each extracted entity according to standardized medical entity types. The entity type determination may influence subsequent knowledge graph access operations by identifying which knowledge repositories contain relevant contextual information for specific entity categories. Different entity types may require different knowledge graph sources and query strategies based on the structure and organization of the corresponding knowledge graph systems. The determination may involve analyzing entity attributes and/or relationships between entities, medical terminology classifications, and contextual indicators to assign appropriate entity type labels.

As further shown in FIG. 1, step 130 may be followed by step 140 of accessing a knowledge graph corresponding to the determined type of medical entity, wherein the knowledge graph contains structured medical knowledge relevant to the type of medical entity. The knowledge graph access operation may involve selecting appropriate knowledge repositories based on the entity types identified during the determination step. The knowledge graph may include ICD-10 Clinical Modification codes, SNOMED CT codes, or other standardized medical coding systems organized in hierarchical structures or semantic relationship networks. The accessing operation may utilize various query languages including SQL for relational database implementations, Cypher for property graph systems, SPARQL for RDF-based knowledge sources, or custom programming interfaces for specialized medical knowledge representations. The knowledge graph access may operate in a simple mode where actions are predetermined based on entity types, or in a complex mode where AI agent-based language models formulate queries dynamically.

Referring again to FIG. 1, step 140 may be followed by step 150 of generating an augmented context by retrieving relevant information from the knowledge graph based on the identified medical entities. The augmented context generation may involve systematic retrieval of medical knowledge that provides contextual information, hierarchical relationships, and domain-specific details related to the extracted medical entities. The retrieval process may execute knowledge graph queries that obtain comprehensive code hierarchies, related diagnostic classifications, clinical criteria, and semantic relationships associated with the identified entities. The retrieved information may be processed and formatted to create structured contextual additions that can be effectively integrated into augmented language model prompts. The augmented context may include diagnostic code options organized in hierarchical order, medication information according to therapeutic classifications, or laboratory test details structured to include test specifications and reference ranges.

As shown in FIG. 1, step 150 may be followed by step 160 of reprocessing the medical note with the large language model using the augmented context to generate enhanced medical analysis results. The reprocessing operation may utilize augmented prompts that combine the original medical documentation with the retrieved knowledge graph information to enable more accurate and comprehensive analysis. The enhanced processing may leverage the contextual information to make more informed decisions about medical entity classification, diagnostic coding selection, and clinical relationship identification. The language model may analyze the clinical details in the original medical note in conjunction with the specific diagnostic classifications, medication information, or laboratory test details provided in the augmented context. The enhanced medical analysis results may include medical coding verification, diagnosis accuracy checking, or consistency validation that demonstrates improved specificity and clinical relevance compared to initial analysis results or to any other information in the patient's EHR.

With continued reference to FIG. 1, step 160 may be followed by step 170 of responding to the enhanced medical analysis results. The responding may include generating alerts, checking for missing laboratory test results based on diagnosis codes and their relationships in the knowledge graph, or determining quality and accuracy of a medical file of a patient. The response operations may involve evaluation of the enhanced analysis results to identify situations that warrant clinical attention, documentation review, or corrective actions. The responding step may generate structured outputs that present analysis findings in formats suitable for clinical review and decision-making, including summaries of identified medical entities, coding recommendations, consistency assessments, and potential issues organized according to clinical priority and significance.

The method 100 may provide technical benefits including improved coding accuracy through the integration of structured medical knowledge with natural language processing capabilities. The knowledge graph augmentation may enable more precise diagnostic code selection by providing hierarchical code structures and clinical criteria that support accurate classification of medical conditions. The method 100 may reduce processing time compared to manual review processes by automating the analysis of medical documentation and providing immediate feedback about potential coding errors or diagnostic inconsistencies. Enhanced clinical decision support capabilities may result from the systematic verification of documented diagnoses against expected laboratory tests and clinical findings, enabling healthcare providers to identify potential gaps in diagnostic workup or documentation quality issues during patient encounters.

Referring to FIG. 1, the step 110 of receiving a medical note containing medical information may involve comprehensive data ingestion capabilities that accommodate diverse medical documentation formats and sources. The receiving operation may utilize data interfaces configured to accept structured electronic health record data, unstructured text documents, and standardized medical messaging formats such as HL7 FHIR or CDA documents. The data interfaces may include application programming interfaces that enable direct integration with electronic health record systems, file upload mechanisms for batch processing of medical documents, and real-time streaming interfaces for continuous processing of medical documentation as documents are created or modified within healthcare information systems.

The receiving step may include document validation operations that verify the integrity and completeness of medical documentation before processing. Document validation may involve format verification to ensure that received documents conform to expected data structures, content validation to confirm the presence of required medical information fields, and encoding verification to ensure proper character encoding and text formatting. The validation process may identify and flag documents that contain formatting errors, incomplete information, or encoding issues that could affect subsequent language model processing operations.

Format standardization operations within the step 110 may convert diverse medical document formats into consistent data structures suitable for language model analysis. The standardization process may extract text content from structured formats such as XML or JSON documents, normalize character encodings to ensure consistent text representation, and segment document content into logical sections such as chief complaint, history of present illness, assessment, and plan. Content preparation may involve removing formatting artifacts, standardizing medical abbreviations, and organizing document sections to facilitate systematic analysis by natural language processing components.

As shown in FIG. 1, the step 120 of processing the medical note with a large language model to identify medical entities within the medical note may utilize advanced natural language processing techniques specifically adapted for medical terminology and clinical documentation patterns. The processing operation may employ transformer-based language model architectures that have been trained on medical literature, clinical documentation datasets, and healthcare-specific terminology to develop domain-aware text analysis capabilities. The language model may utilize attention mechanisms that focus on medically relevant portions of text and contextual understanding capabilities that recognize relationships between different medical concepts within clinical documentation.

The large language model may implement named entity recognition techniques that systematically identify and classify medical entities within unstructured clinical text. The entity recognition process may utilize pattern matching algorithms that identify medical terminology based on lexical patterns, contextual analysis that considers surrounding text to disambiguate medical terms, and confidence scoring mechanisms that assess the reliability of entity identification and classification operations. The language model may recognize various forms of medical expression including formal medical terminology, clinical abbreviations, colloquial references to medical conditions, and specialty-specific notation systems used in different clinical domains.

Medical entity extraction operations within the step 120 may identify diagnoses that include disease conditions, medical disorders, and clinical findings documented within patient notes. Diagnosis extraction may recognize diagnostic statements through various linguistic patterns including direct diagnostic assertions such as “patient diagnosed with diabetes mellitus,” differential diagnosis discussions that consider multiple possible conditions, and provisional diagnostic considerations based on symptom presentations. The extraction process may identify both explicit diagnostic statements and implied diagnostic considerations based on symptom descriptions and clinical presentations documented within medical notes.

The processing operation may extract medications that include pharmaceutical substances, dosages, administration routes, and treatment regimens documented in medical notes. Medication extraction may identify drug names through recognition of generic and brand name variations, pharmaceutical classifications, and therapeutic categories. The language model may recognize medication entities through various forms including complete drug names, medical abbreviations for common medications, and dosage specifications that include strength, frequency, and duration information. Medication extraction may associate identified pharmaceutical substances with indication information linking prescribed medications to specific diagnostic conditions or treatment objectives.

Laboratory results extraction within the step 120 may identify test values, measurement units, reference ranges, and clinical interpretations documented in medical notes or laboratory reports. The language model may recognize numerical values associated with specific laboratory tests, qualitative results such as positive or negative findings, and clinical assessments that interpret laboratory findings in relation to patient conditions. Laboratory result entities may include both structured data from laboratory information systems and unstructured result descriptions embedded within clinical notes. The extraction process may identify temporal information associated with laboratory results to enable tracking of test result trends and monitoring of patient condition changes over time.

Medical procedures extraction may identify medical interventions, surgical procedures, diagnostic procedures, and therapeutic treatments documented within medical notes. The language model may recognize procedure entities through procedure names, medical intervention descriptions, and treatment modality references. Procedure extraction may include identification of anatomical locations where procedures are performed, procedural techniques or approaches utilized, and clinical outcomes associated with medical interventions. The processing operation may classify extracted procedures according to procedural complexity, clinical specialties, and billing classifications used in healthcare reimbursement systems.

The method 100 may process historical patient documents to generate summaries of historical and current medical information relevant to upcoming physician visits. Historical document processing may involve analysis of previous medical encounters, diagnostic assessments, treatment histories, and clinical outcomes to create comprehensive patient summaries that support continuity of care. The processing operation may identify patterns in historical medical documentation, track changes in patient conditions over time, and highlight relevant clinical information that may inform current treatment decisions. Historical document analysis may generate structured summaries that organize patient information according to medical specialties, chronological timelines, or clinical significance to support physician preparation for patient encounters.

With continued reference to FIG. 1, the step 130 of determining a type of medical entity from the identified medical entities may involve comprehensive analysis of entity characteristics and classifications to categorize each extracted entity according to standardized medical entity types. The determination process may analyze linguistic patterns, contextual indicators, and semantic features associated with each identified medical entity to assign appropriate classification labels that correspond to predefined medical entity categories. The entity type determination may utilize classification algorithms that evaluate textual features, medical terminology patterns, and contextual relationships to distinguish between different categories of medical information such as diagnoses, medications, laboratory results, and medical procedures.

The determination process may analyze entity characteristics including lexical patterns that indicate specific medical entity types based on terminology structure and medical naming conventions. Diagnostic entities may be identified through characteristic patterns such as disease terminology, anatomical references, and clinical condition descriptors that distinguish diagnostic information from other types of medical entities. Medication entities may be recognized through pharmaceutical naming patterns, drug classification indicators, and dosage-related terminology that differentiate medication information from other clinical content. Laboratory result entities may be distinguished through numerical value patterns, measurement unit indicators, and test name conventions that characterize laboratory data within medical documentation.

As further shown in FIG. 1, the entity type determination may involve contextual analysis that examines surrounding text and document structure to provide additional classification cues for ambiguous medical entities. The contextual analysis may evaluate sentence structure, paragraph organization, and document section headings to determine the clinical context in which medical entities appear. Medical entities appearing in assessment sections may be more likely to represent diagnostic information, while entities documented in medication reconciliation sections may correspond to pharmaceutical substances. Laboratory entities may be identified through association with numerical values, reference ranges, and temporal indicators that characterize laboratory test results and clinical measurements.

The determination process may utilize confidence scoring mechanisms that assess the reliability of entity type classifications based on the strength of linguistic and contextual indicators. Confidence scores may reflect the degree of certainty associated with each entity type assignment, enabling subsequent processing operations to prioritize high-confidence classifications and flag uncertain determinations for additional review. The scoring mechanism may consider multiple classification features including lexical pattern strength, contextual consistency, and agreement between different classification approaches to generate comprehensive confidence assessments for each medical entity type determination.

The step 130 may influence subsequent knowledge graph access operations by identifying which knowledge repositories contain relevant contextual information for specific entity categories. Different medical entity types may correspond to different knowledge graph systems based on the specialized medical knowledge domains represented within each knowledge repository. Diagnostic entities may trigger access to diagnostic coding knowledge graphs such as ICD-10 Clinical Modification or SNOMED CT systems or other systems that contain hierarchical diagnostic classifications and clinical criteria. Medication entities may direct the system to pharmaceutical knowledge graphs that include drug databases, therapeutic classifications, and medication interaction information.

Laboratory result entities identified during the determination process may correspond to laboratory knowledge graphs that contain test specifications, reference ranges, and clinical interpretation guidelines. Procedure entities may be associated with procedural knowledge graphs that include medical intervention classifications, anatomical specifications, and clinical outcome data. The entity type determination may enable the system to select appropriate knowledge graph sources and formulate corresponding query strategies that are optimized for specific types of medical information and knowledge representation structures.

The determination process may support multi-entity classification scenarios where individual text segments contain multiple types of medical entities that require separate classification and knowledge graph access operations. Complex medical statements may include diagnostic information combined with medication prescriptions, laboratory test orders, and procedural recommendations that each correspond to different entity types and knowledge graph systems. The determination process may identify and separate these multiple entity types to enable appropriate knowledge graph access for each category of medical information within the same medical documentation segment.

Entity type determination may utilize machine learning classification models that have been trained on annotated medical documentation datasets to recognize patterns associated with different medical entity categories. The classification models may incorporate feature extraction techniques that identify relevant textual and contextual characteristics for medical entity type determination. Training datasets may include diverse medical documentation types spanning multiple clinical specialties to ensure broad applicability of entity type classification capabilities across different healthcare domains and documentation styles.

Referring to FIG. 1, the step 140 of accessing a knowledge graph corresponding to the determined type of medical entity may involve systematic selection and querying of appropriate knowledge repositories based on the entity types identified during the determination step. The knowledge graph access operation may utilize the entity type classifications to identify which knowledge repositories contain relevant contextual information for specific entity categories. The accessing operation may involve establishing connections to knowledge graph systems, formulating appropriate queries based on identified medical entities, and retrieving structured medical knowledge that provides contextual information to support enhanced language model analysis.

As shown in FIG. 1, the knowledge graph may contain structured medical knowledge relevant to the type of medical entity organized in hierarchical structures or semantic relationship networks that enable systematic querying and retrieval of contextual information. The structured medical knowledge may include comprehensive medical coding systems, clinical terminology databases, and semantic relationship networks that represent connections between different medical concepts. The knowledge graph organization may facilitate efficient access to related medical information through hierarchical navigation, semantic relationship traversal, and cross-referencing operations between different medical knowledge domains.

The knowledge graph may comprise ICD-10 Clinical Modification codes that provide diagnostic classifications used for medical billing and clinical documentation purposes. ICD-10 Clinical Modification knowledge graphs may organize diagnostic codes in hierarchical structures where broader diagnostic categories are subdivided into more specific conditions and anatomical locations. The ICD-10-CM knowledge graph may include diagnostic codes such as M06 for rheumatoid arthritis as a general category, with more specific subcategories including M06.0 for rheumatoid arthritis without rheumatoid factor and M06.062 for rheumatoid arthritis without rheumatoid factor affecting the left knee. The hierarchical organization may enable the system to traverse diagnostic code trees to identify related conditions, parent categories, and more specific subcategories based on clinical presentations documented in medical notes.

With continued reference to FIG. 1, the knowledge graph may comprise SNOMED CT codes that provide comprehensive clinical terminology systems representing medical concepts and their relationships in detailed semantic structures. SNOMED CT knowledge graphs may organize medical concepts using multiple relationship types including hierarchical classifications, part-whole relationships, and clinical associations between different medical entities. The SNOMED CT knowledge graph may include concepts such as “Seronegative rheumatoid arthritis of left knee joint (disorder)” with semantic identifiers that provide detailed clinical descriptions and relationship mappings to related procedures, laboratory tests, and clinical findings. Relationship mappings within SNOMED CT knowledge graphs may connect diagnostic concepts with associated laboratory procedures through semantic relationships such as “Interprets” relationships that link diagnostic conditions to relevant laboratory tests.

The knowledge graph may comprise other standardized medical coding systems including CPT procedure codes, LOINC laboratory test codes, RxNorm medication identifiers, and additional medical classification frameworks used in healthcare environments. These standardized medical coding systems may provide specialized knowledge domains that support different aspects of medical documentation analysis including procedural coding verification, laboratory test validation, and medication classification. The standardized coding systems may be organized in hierarchical structures that enable systematic navigation of medical classifications and cross-referencing operations between different coding frameworks.

As further shown in FIG. 1, the knowledge graphs may include Wikidata knowledge graphs containing broader medical knowledge including disease relationships, epidemiological information, and connections between different medical conditions. Wikidata knowledge graphs may represent diseases as entities with associated properties including symptoms, risk factors, related conditions, and differential diagnosis considerations. The Wikidata knowledge graph may store relationships between diseases and their associated diagnostic codes, enabling cross-referencing between different coding systems and knowledge representations. Disease relationship queries within Wikidata knowledge graphs may identify conditions that share similar symptoms or clinical presentations, supporting differential diagnosis processes and clinical reasoning operations.

The knowledge graphs may be accessed through Python code support for custom knowledge graph implementations and specialized medical databases that utilize proprietary data formats or custom query interfaces. Custom Python interfaces may accommodate unique medical knowledge representations developed for specific clinical domains or research applications. Python code implementations may integrate multiple medical data sources, perform real-time data aggregation, or provide specialized analytical capabilities that extend beyond standard query language functionality.

The system may include database systems that provide structured storage using commercial database management systems such as Oracle Database, Microsoft SQL Server, IBM Db2, PostgreSQL, nosql DSs such as MongoDB or MariaDB. Database systems may organize medical knowledge in relational table structures that can be efficiently queried using SQL syntax for accessing diagnostic code hierarchies, medication databases, and laboratory test classifications. The database implementations may utilize indexing strategies and query optimization techniques to support rapid retrieval of medical knowledge during real-time documentation analysis operations. Commercial database management systems may provide enterprise-grade reliability, scalability, and security features that support healthcare information processing requirements.

The system may include graph database systems using Neo4j, Amazon Neptune, or Azure Cosmos DB for specialized storage and query capabilities for property graph knowledge representations. Graph database systems may represent medical concepts as nodes with associated properties and relationships as edges that connect related medical entities. The graph structure may enable complex traversal operations that explore multi-hop relationships between medical concepts, diagnostic codes, and clinical procedures through relationship paths. Graph database implementations may utilize Cypher query language for accessing property graph structures and performing complex relationship traversal operations that support clinical reasoning and knowledge exploration tasks.

The accessing operation may utilize various query languages including SQL for relational database implementations of medical coding systems, Cypher for property graph systems, SPARQL for RDF-based knowledge sources such as Wikidata, or custom programming interfaces for specialized medical knowledge representations. Query language selection may be determined automatically based on the type of knowledge graph being accessed or configured based on organizational preferences and system architecture requirements. The multi-language query capability may enable the system to access diverse medical knowledge sources and integrate information from multiple knowledge representation systems without requiring standardization of underlying data storage architectures.

Referring to FIG. 1, the simple mode operation may provide a streamlined approach for accessing knowledge graphs through predetermined actions that are automatically triggered based on the type of medical entity identified during the entity determination process. The simple mode may utilize template-based query systems that execute predetermined knowledge graph interactions according to predefined rules that specify which knowledge graph queries should be executed based on the characteristics of medical entities extracted from clinical documentation. The predetermined approach may eliminate the computational overhead associated with dynamic query generation while ensuring consistent and reliable access to contextual medical knowledge through optimized query patterns designed for common medical documentation analysis scenarios.

The simple mode may operate through predefined rules that automatically trigger specific knowledge graph queries when particular types of medical entities are identified during the initial language model processing phase. The predefined rules may specify that diagnostic entity identification should result in execution of diagnostic code hierarchy queries, medication entity identification should trigger pharmaceutical database access operations, and laboratory result entity identification should initiate laboratory test specification retrieval processes. The rule-based triggering mechanism may provide predictable processing behavior that supports real-time medical documentation analysis in clinical environments where consistent response times and reliable analytical outcomes are required for effective clinical workflow integration.

As shown in FIG. 1, the simple mode may utilize predefined query templates that are automatically populated with extracted medical entity values to generate specific database queries or knowledge graph access operations. The predefined query templates may include placeholder variables that are replaced with actual medical entity values during the template-filling process, enabling efficient access to relevant medical knowledge without requiring complex query formulation capabilities. A template query for diagnostic code expansion may include a placeholder for the diagnostic code value that is automatically substituted with the specific code identified by the language model analysis, such as “expand_tree (type=icd10 cm, root={icd10_code})” where the placeholder variable is replaced with the extracted diagnostic code.

The template-based approach may provide computational efficiency by avoiding complex query formulation processes that would otherwise require sophisticated reasoning capabilities and dynamic query generation algorithms. The predefined nature of the query templates may enable optimization of query performance through pre-compiled query structures, indexed database access patterns, and cached query execution plans that reduce processing latency during real-time medical documentation analysis operations. The template-filling approach may maintain analytical accuracy through the use of optimized query patterns that have been specifically designed and validated for common medical documentation analysis scenarios across different clinical specialties and documentation types.

With continued reference to FIG. 1, the automatic population of predefined query templates with extracted medical entity values may involve systematic substitution processes that replace template placeholder variables with specific entity identifiers, medical codes, or terminology values identified during the language model analysis phase. The substitution process may validate entity values against expected data formats and value ranges to ensure that populated queries conform to knowledge graph system requirements and data integrity constraints. Template population may include data type conversion operations that transform extracted entity values into appropriate formats for specific knowledge graph query languages and database access protocols.

The predefined query templates may be stored within the system as structured query patterns that accommodate different types of medical entities and knowledge graph systems through parameterized query structures. Diagnostic entity templates may include patterns for hierarchical code expansion, related condition identification, and clinical criteria retrieval operations that are commonly required for diagnostic coding verification and accuracy assessment. Medication entity templates may include patterns for pharmaceutical information retrieval, therapeutic classification access, and drug interaction checking operations that support medication verification and clinical decision support processes.

As further shown in FIG. 1, the simple mode operation may support template-based query execution against multiple knowledge graph systems through unified template interfaces that abstract the underlying query language differences and data access methods. The template system may include query patterns for SQL-based relational database access, Cypher-based property graph queries, SPARQL-based semantic web access, and custom API-based knowledge graph interactions. The unified template approach may enable consistent query formulation and execution across diverse knowledge graph implementations without requiring specialized knowledge of each query language or data format.

The template-based query system may include optimization mechanisms that improve query execution performance through pre-compiled query structures, connection pooling for database access operations, and result caching for frequently accessed medical knowledge. Query optimization may reduce processing latency by maintaining prepared query statements that can be rapidly executed with different parameter values, establishing persistent connections to knowledge graph systems to eliminate connection overhead, and caching commonly accessed medical knowledge to reduce redundant database queries during high-volume processing operations.

Referring again to FIG. 1, the simple mode may provide deterministic processing behavior that enables predictable analysis outcomes and consistent processing times for routine medical documentation analysis tasks. The predetermined nature of the query operations may eliminate variability associated with dynamic query formulation and enable accurate estimation of processing resource requirements for different types of medical documentation. The deterministic approach may support clinical environments that require reliable processing times and consistent analytical outcomes for integration with existing healthcare workflow systems and electronic health record platforms.

The simple mode operation may be particularly suitable for high-volume processing scenarios where medical documentation follows standardized formats and the required analysis operations can be anticipated based on the medical documentation type or clinical context. Clinical environments that process large numbers of similar medical notes, routine diagnostic coding tasks, or standardized documentation updates may benefit from the computational efficiency and predictable processing behavior provided by the template-based approach. The reduced computational requirements of the simple mode may enable real-time processing of medical documentation in resource-constrained environments or systems handling concurrent analysis requests from multiple clinical users.

Referring to FIG. 1, the complex mode operation may provide advanced analytical capabilities where an AI agent-based large language model formulates queries to access the knowledge graph as a tool through dynamic reasoning processes that adapt to specific clinical contexts and documentation complexity. The complex mode may employ sophisticated AI agent-based processing approaches that enable language model AI agents to determine appropriate query strategies, select relevant knowledge graph sources, and adapt the analysis process based on intermediate results obtained during multi-step analytical operations. The AI agent-based approach may utilize reasoning frameworks that combine reasoning and action capabilities to support complex clinical decision-making processes that require iterative interaction with knowledge graph systems and adaptive query formulation based on evolving analytical requirements.

The AI agent-based large language model may utilize a ReAct framework that enables language model AI agents to alternate between reasoning steps and action execution phases during complex medical analysis tasks. The ReAct framework may provide structured approaches for combining reasoning and action capabilities, enabling AI agents to formulate analytical plans based on medical documentation content, execute knowledge graph queries as tools to retrieve relevant medical knowledge, and reason about retrieved information to support sophisticated clinical assessments. During reasoning phases, AI agents may analyze medical documentation to identify relevant clinical questions, evaluate potential knowledge graph sources, and formulate plans for knowledge graph exploration and verification processes that address specific analytical objectives.

As shown in FIG. 1, the ReAct framework may support action phases that involve execution of specific knowledge graph queries, retrieval of medical knowledge from selected repositories, and processing of query results to inform subsequent reasoning steps. The iterative nature of the ReAct framework may enable multi-step analysis processes where AI agents refine their understanding through successive cycles of reasoning and knowledge graph interaction. The framework may enable AI agents to modify their analytical approach based on intermediate results, pursue alternative query strategies when initial approaches yield insufficient information, and integrate information from multiple knowledge graph sources to develop comprehensive clinical assessments.

The AI agent-based large language model may utilize LangChain libraries that provide comprehensive frameworks for building AI agent-based medical analysis systems that integrate language models with knowledge graph tools and external medical resources. LangChain libraries may offer pre-built components for AI agent construction, tool integration, and workflow management that simplify the development of complex medical analysis applications. The LangChain framework may provide unified interfaces that enable language model AI agents to interact with diverse knowledge graph types including relational databases, property graphs, and semantic web resources without requiring specialized knowledge of each query language or data format.

With continued reference to FIG. 1, the LangChain libraries may support tool interfaces that abstract the underlying query languages and data access methods used by different knowledge graph systems. The tool interfaces may enable AI agents to formulate queries using natural language descriptions of information requirements, which are then automatically translated into appropriate query languages such as SQL for relational databases, Cypher for property graphs, or SPARQL for semantic web resources. The abstraction provided by LangChain tool interfaces may enable AI agents to focus on clinical reasoning and analytical logic rather than technical details of knowledge graph access and query formulation.

The AI agent-based approach may support complex reasoning chains where initial analysis results inform subsequent query operations and analytical steps through iterative refinement processes. Complex reasoning chains may begin with preliminary analysis of medical documentation to identify potential clinical issues or analytical objectives, followed by targeted knowledge graph queries that retrieve relevant medical knowledge to address specific questions or verify particular clinical relationships. Subsequent reasoning steps may analyze the retrieved knowledge in conjunction with the original medical documentation to identify additional analytical requirements, formulate follow-up queries, or integrate information from multiple knowledge sources to develop comprehensive clinical assessments.

As further shown in FIG. 1, the complex reasoning chains may enable AI agents to pursue differential diagnosis analysis by exploring alternative diagnostic possibilities based on documented symptoms and clinical findings. AI agents may formulate initial queries to retrieve diagnostic codes associated with documented symptoms, analyze the retrieved diagnostic options to identify potential differential diagnoses, and execute additional queries to explore clinical criteria and distinguishing features associated with different diagnostic possibilities. The reasoning chain may continue with queries to identify expected laboratory tests or clinical findings associated with different diagnostic options, enabling comprehensive differential diagnosis analysis that considers multiple clinical possibilities and supporting evidence.

The AI agent-based large language model may utilize multiple types of specialized AI agents that are configured to perform distinct analytical tasks within the medical documentation analysis workflow. Diagnostic coding AI agents may focus on accurate assignment and verification of diagnostic codes from medical documentation through specialized training on diagnostic terminology, coding guidelines, and clinical criteria. The diagnostic coding AI agents may be configured to analyze clinical descriptions, identify relevant diagnostic entities, and select appropriate ICD-10-CM or SNOMED CT codes based on documented clinical presentations. Diagnostic coding AI agents may interact with diagnostic code knowledge graphs to retrieve hierarchical code structures and verify the specificity and accuracy of code assignments through systematic comparison of clinical documentation with coding criteria.

Medication verification AI agents may specialize in analyzing medication-related information within medical documentation to verify prescription accuracy, identify potential drug interactions, and detect potentially incorrect medical aspects (e.g. drugs, procedures, diagnoses etc.) by using KGs and Standard Treatment Guidelines”s. The medication verification AI agents may access pharmaceutical knowledge graphs to retrieve medication information including therapeutic classifications, dosage guidelines, contraindications, and interaction data. Medication verification AI agents may compare documented prescriptions against patient diagnoses to verify therapeutic appropriateness and identify potential prescribing errors or contraindications that warrant clinical review and possible medication adjustments.

With continued reference to FIG. 1, laboratory analysis AI agents may focus on verification of laboratory test orders and interpretation of laboratory results in relation to documented diagnoses. The laboratory analysis AI agents may interact with laboratory knowledge graphs to identify expected tests based on diagnostic codes, verify the presence of appropriate laboratory data, and detect missing or contradictory test results. Laboratory analysis AI agents may be trained to recognize relationships between specific diagnoses and associated laboratory procedures through semantic relationship exploration in medical knowledge graphs, enabling systematic verification of diagnostic consistency and identification of potential gaps in clinical assessment.

Consistency validation AI agents may perform comprehensive cross-checking operations across different elements of medical documentation to identify potential inconsistencies or contradictions. The consistency validation AI agents may analyze relationships between diagnoses, medications, laboratory results, and procedures to detect logical inconsistencies or missing clinical information. Consistency validation AI agents may utilize multiple knowledge graph sources simultaneously to perform comprehensive validation operations that span different medical knowledge domains, enabling detection of subtle inconsistencies that may not be apparent through single-domain analysis approaches.

Differential diagnosis AI agents may support clinical reasoning processes by exploring alternative diagnostic possibilities based on documented symptoms and clinical findings. The differential diagnosis AI agents may query disease knowledge graphs to identify conditions with similar clinical presentations and retrieve comparative diagnostic criteria. Differential diagnosis AI agents may be configured to perform multi-hop reasoning operations that explore complex relationships between symptoms, diseases, and diagnostic tests across multiple knowledge graph systems, enabling comprehensive exploration of diagnostic alternatives and supporting evidence.

As shown in FIG. 1, the specialized AI agents may be trained using supervised learning approaches with annotated medical documentation datasets that include correct coding assignments, verified clinical relationships, and validated analytical outcomes. Training datasets may include diverse medical documentation types spanning multiple clinical specialties to ensure broad applicability of trained AI agents across different healthcare domains and documentation styles. The supervised training process may expose AI agents to various documentation styles, clinical abbreviations, and specialty-specific terminology to develop robust analytical capabilities that can handle diverse clinical scenarios and documentation patterns.

The AI agent training may utilize reinforcement learning techniques where AI agents receive feedback based on the accuracy of their analytical outputs and the clinical appropriateness of their recommendations. AI agents may receive positive reinforcement when their analytical results match validated clinical assessments and negative feedback when their outputs contain errors or inconsistencies. The reinforcement learning process may enable AI agents to develop optimal query strategies for accessing knowledge graphs and refine their reasoning approaches based on accumulated experience with different types of medical documentation and clinical scenarios.

With continued reference to FIG. 1, the AI agent training may employ multi-task training approaches that enable AI agents to develop capabilities across multiple related analytical tasks simultaneously by training on diverse medical documentation analysis objectives. Multi-task training may improve AI agent generalization capabilities and enable more efficient knowledge transfer between related analytical tasks such as diagnostic coding, medication verification, and laboratory analysis. AI agents trained using multi-task approaches may demonstrate improved performance on complex analytical scenarios that require coordination of multiple analytical capabilities and integration of information from diverse medical knowledge domains.

The AI agent-based approach may provide enhanced analytical flexibility compared to predetermined template-based processing by enabling dynamic adaptation to complex clinical scenarios that require sophisticated reasoning processes. The AI agent-based system may handle unusual clinical presentations, complex multi-system disorders, or ambiguous clinical findings through adaptive analytical capabilities that can explore multiple knowledge graph sources and pursue alternative reasoning strategies based on intermediate analytical results. The dynamic query formulation capabilities may enable AI agents to access specialized medical knowledge repositories and pursue complex analytical pathways that would not be possible through predetermined template-based approaches.

Referring to FIG. 1, the step 150 of generating an augmented context by retrieving relevant information from the knowledge graph based on the identified medical entities may involve systematic retrieval of medical knowledge that provides contextual information, hierarchical relationships, and domain-specific details related to the extracted medical entities. The augmented context generation may transform the original medical note analysis by incorporating structured medical knowledge that may not be available to the language model through training data alone and are constantly updated.

The retrieval process may execute knowledge graph queries that obtain comprehensive code hierarchies, related diagnostic classifications, clinical criteria, and semantic relationships associated with the identified entities to create enhanced analytical capabilities.

The augmented context generation may utilize the identified medical entities to formulate knowledge graph queries that retrieve comprehensive medical knowledge surrounding the initially identified medical conditions, medications, laboratory tests, or procedures. The retrieval operations may access hierarchical code structures from medical coding knowledge graphs such as ICD-10 Clinical Modification or SNOMED CT systems to provide detailed classification options that correspond to specific clinical presentations documented in the original medical documentation. The query formulation process may analyze the entity type, extracted terminology, and contextual information to determine appropriate knowledge graph sources and query strategies for retrieving relevant contextual information.

As shown in FIG. 1, the systematic retrieval of medical knowledge may involve hierarchical expansion queries that retrieve comprehensive code trees and classification structures surrounding initially identified medical entities. The hierarchical expansion operations may start with general diagnostic codes, medication categories, or procedure classifications identified during initial analysis and retrieve all related subcategories, specific classifications, and hierarchical relationships. The hierarchical expansion may provide comprehensive options for selecting appropriate specific codes based on clinical details present in the original medical documentation, enabling more precise medical entity classification and coding assignments.

The augmented context generation may include cross-referencing queries that retrieve relationships between different medical coding systems and knowledge representations to provide comprehensive contextual information spanning multiple medical knowledge domains. The cross-referencing process may map ICD-10-CM diagnostic codes to corresponding SNOMED CT concepts, link diagnostic entities to associated laboratory tests, or connect medication entities to therapeutic indications and contraindications. Cross-referencing operations may enable the augmentation process to provide multi-dimensional contextual information that supports comprehensive clinical analysis and verification operations.

With continued reference to FIG. 1, the retrieval process may include semantic relationship queries that explore conceptual connections between medical entities and related clinical concepts stored in semantic knowledge graphs and medical ontologies. The semantic relationship exploration may identify diseases with similar clinical presentations, medications with comparable therapeutic effects, or laboratory tests that provide related clinical information. Semantic relationship queries may enhance the contextual information by providing differential diagnosis considerations, alternative treatment options, or complementary diagnostic assessments that support comprehensive clinical reasoning operations.

The retrieved knowledge graph information may be processed and formatted to create structured contextual additions that can be effectively integrated into augmented language model prompts. The formatting process may organize the retrieved medical knowledge according to relevance, hierarchical relationships, and clinical significance to support optimal language model comprehension and utilization. The structured formatting may present diagnostic code options in hierarchical order, medication information according to therapeutic classifications, or laboratory test details organized by clinical significance to enable systematic utilization during enhanced analysis operations.

As further shown in FIG. 1, the contextual information organization may group related medical knowledge elements to provide coherent and logically structured augmentation content. Diagnostic code hierarchies may be presented with clear parent-child relationships and anatomical specifications that enable precise code selection based on documented clinical presentations. Medication information may be organized according to therapeutic categories, dosage forms, and clinical indications to support accurate medication verification and therapeutic appropriateness assessment. Laboratory test context may be structured to include test specifications, reference ranges, and clinical interpretation guidelines in formats that support accurate analysis and verification operations.

The augmented context generation may include relevance filtering processes that prioritize pertinent knowledge graph information based on specific clinical context and analytical objectives of the medical documentation analysis. The filtering process may select relevant diagnostic code options based on anatomical locations mentioned in the medical note, prioritize medication information based on documented clinical conditions, or emphasize laboratory test details that correspond to suspected diagnostic considerations. Relevance filtering may ensure that the augmented context provides focused and clinically appropriate information without overwhelming the language model with excessive or irrelevant details that could affect analytical accuracy.

Referring again to FIG. 1, the augmented prompt generation may combine the original medical documentation with the retrieved and formatted knowledge graph information to create comprehensive analytical inputs for enhanced language model processing. The prompt generation process may integrate the contextual information in a manner that clearly distinguishes between the original clinical content and the supplementary knowledge graph information while maintaining logical flow and analytical coherence. The augmented prompts may include explicit instructions for utilizing the contextual information to improve analytical accuracy and specificity during subsequent language model processing operations.

The augmented prompt structure may present the original medical note followed by clearly labeled contextual sections that contain the retrieved knowledge graph information organized according to medical entity types and clinical relevance. Diagnostic context sections may include hierarchical code listings with descriptions and specifications that enable precise diagnostic classification. Medication context sections may provide pharmaceutical details and therapeutic information that support medication verification and appropriateness assessment. Referring to FIG. 1, the step 160 of reprocessing the medical note with the large language model using the augmented context to generate enhanced medical analysis results may utilize augmented prompts that combine the original medical documentation with the retrieved knowledge graph information to enable more accurate and comprehensive analysis. The reprocessing operation may leverage the contextual information provided through the augmented context to make more informed decisions about medical entity classification, diagnostic coding selection, and clinical relationship identification compared to initial analysis performed without knowledge graph augmentation. The enhanced processing may analyze the clinical details in the original medical note in conjunction with the specific diagnostic classifications, medication information, or laboratory test details provided in the augmented context to generate results that demonstrate improved specificity and clinical relevance.

The enhanced processing may utilize the augmented prompts to perform more accurate medical entity classification by leveraging the structured medical knowledge retrieved from knowledge graph systems. The language model may analyze the original clinical descriptions in conjunction with hierarchical diagnostic code options, pharmaceutical classifications, or laboratory test specifications provided in the augmented context to select more precise entity classifications. The contextual information may enable the language model to resolve ambiguities in medical terminology, distinguish between similar medical conditions based on specific clinical criteria, and assign more accurate entity labels based on the comprehensive medical knowledge provided through knowledge graph augmentation.

As shown in FIG. 1, the enhanced processing may improve diagnostic coding selection by utilizing hierarchical code structures and clinical criteria retrieved from diagnostic knowledge graphs. The language model may analyze the clinical details documented in the original medical note and match them with specific diagnostic classifications provided in the augmented context to select codes that accurately represent the documented clinical conditions. The diagnostic coding selection process may consider anatomical locations, clinical presentations, diagnostic modifiers, and severity indicators provided through the knowledge graph information to identify the most appropriate specific diagnostic codes from the hierarchical options available in the augmented context.

The reprocessing operation may enhance clinical relationship identification by utilizing semantic relationships and cross-referencing information retrieved from knowledge graph systems. The language model may identify connections between documented diagnoses and associated medications, laboratory tests, or procedures based on the relationship information provided in the augmented context. The clinical relationship identification may enable the system to verify the consistency of medical documentation by confirming that documented treatments align with established clinical protocols, that laboratory tests correspond to documented diagnoses, and that prescribed medications are appropriate for documented conditions.

With continued reference to FIG. 1, the enhanced medical analysis results may comprise medical coding verification that evaluates the accuracy and specificity of diagnostic codes assigned to documented clinical conditions. The medical coding verification may compare the clinical descriptions in the original medical note with the coding hierarchies, ontology relationships, and semantic mappings and hierarchical classifications provided through knowledge graph augmentation to identify situations where more specific codes are available or where assigned codes do not accurately reflect the documented clinical presentation. The verification process may generate recommendations for more appropriate diagnostic codes along with explanations of the clinical factors that support the suggested code revisions based on the structured medical knowledge retrieved from knowledge graph systems.

The enhanced medical analysis results may comprise diagnosis accuracy checking that evaluates the consistency between documented diagnoses and available clinical evidence including laboratory test results, medication prescriptions, and clinical findings. The diagnosis accuracy checking may utilize the cross-coding mapping capabilities and semantic relationship information retrieved from knowledge graph systems to identify expected clinical evidence for documented diagnoses and verify the presence of supporting information in the medical documentation. The accuracy checking process may identify situations where documented diagnoses lack supporting clinical evidence, where laboratory results contradict documented diagnostic classifications, or where additional clinical assessments may be needed to support or verify specific diagnoses.

As further shown in FIG. 1, the enhanced medical analysis results may comprise consistency validation that performs comprehensive cross-checking operations across different elements of medical documentation to identify potential inconsistencies or contradictions. The consistency validation may analyze relationships between diagnoses, medications, laboratory results, and procedures based on the medical knowledge and relationship information provided through knowledge graph augmentation. The validation process may detect logical inconsistencies such as medications that are contraindicated for documented conditions, laboratory results that do not support documented diagnoses, or missing clinical information that would typically be expected based on documented medical conditions.

The consistency validation may utilize the semantic relationships and clinical criteria retrieved from knowledge graph systems to identify expected associations between different medical entities and flag situations where these associations are absent or contradictory in the medical documentation. The validation process may compare documented medication prescriptions against therapeutic indications for documented diagnoses, verify that laboratory test orders correspond to appropriate diagnostic workup for documented conditions, and identify missing clinical assessments that would typically be associated with specific medical conditions based on the structured medical knowledge provided through knowledge graph augmentation.

Referring again to FIG. 1, the enhanced medical analysis results may demonstrate improved accuracy and specificity compared to analysis performed without knowledge graph augmentation through more precise diagnostic code selection, enhanced clinical relationship identification, and comprehensive consistency verification. The improved analytical outcomes may result from the systematic incorporation of structured medical knowledge that provides detailed classification criteria, hierarchical code relationships, and semantic associations between different medical concepts. The enhanced results may enable healthcare providers to identify potential documentation quality issues, coding optimization opportunities, and clinical decision support recommendations that may not be apparent through analysis of medical documentation alone.

The enhanced processing may generate confidence scores that indicate the reliability of the analytical results based on the strength of supporting evidence provided through knowledge graph augmentation and the consistency of clinical information across different elements of the medical documentation. The confidence scoring may reflect the degree of certainty associated with diagnostic code assignments, the strength of clinical relationships identified through semantic analysis, and the reliability of consistency validation findings based on the comprehensive medical knowledge retrieved from knowledge graph systems. High confidence scores may indicate strong agreement between documented clinical information and established medical knowledge, while lower confidence scores may flag situations requiring additional clinical review or verification.

With continued reference to FIG. 1, the enhanced medical analysis results may include detailed explanations of analytical logic that reference the specific knowledge graph information and medical relationships that support the generated conclusions and recommendations. The explanatory information may provide healthcare providers with transparent insight into the reasoning processes used to generate analytical results, enabling informed evaluation of system recommendations and supporting clinical decision-making processes. The detailed explanations may include references to specific diagnostic criteria, clinical guidelines, or medical knowledge sources that support the analytical conclusions, providing healthcare providers with the information needed to validate and act upon system recommendations.

The reprocessing operation may support iterative analysis processes where initial enhanced results are further refined through additional processing cycles that incorporate feedback from clinical users or additional knowledge graph information. The iterative approach may enable continuous improvement of analytical accuracy through successive refinement cycles that incorporate new clinical information, updated medical knowledge, or corrections based on clinical feedback. The iterative processing capability may support complex clinical scenarios that require multiple analytical perspectives or comprehensive evaluation of alternative diagnostic or treatment options based on evolving clinical information and knowledge graph resources.

Referring to FIG. 1, the step 170 of responding to the enhanced medical analysis results may provide comprehensive response operations that evaluate the enhanced analysis results to identify situations that warrant clinical attention, documentation review, or corrective actions. The responding step may generate structured outputs that present analysis findings in formats suitable for clinical review and decision-making, including summaries of identified medical entities, coding recommendations, consistency assessments, and potential issues organized according to clinical priority and significance. The response operations may include generating alerts for potential inconsistencies between the medical note content and the knowledge graph information, checking for missing laboratory test results based on diagnosis codes and their relationships in the knowledge graph, and determining quality and accuracy of a patient medical file.

As shown in FIG. 1, the step 170 may include generating alerts for potential inconsistencies between the medical note content and the knowledge graph information through systematic comparison of documented clinical information with established medical knowledge retrieved from knowledge graph systems. The alert generation process may identify situations where documented diagnoses contradict available laboratory test results, where prescribed medications are inappropriate for documented conditions, or where clinical findings are inconsistent with established diagnostic criteria. The inconsistency detection may utilize the cross-coding mapping capabilities and semantic relationship information to compare documented medical information against expected clinical patterns and identify deviations that may indicate documentation errors or clinical issues requiring physician review.

The alert generation may create notifications that are classified according to clinical urgency and potential impact on patient care to ensure that important problems receive appropriate priority. High-priority alerts may be generated for situations involving patient safety concerns, significant diagnostic discrepancies, or missing clinical information that could affect treatment decisions. Lower-priority alerts may address documentation quality issues, coding optimization opportunities, or minor inconsistencies that do not immediately impact patient care but may affect billing accuracy or clinical quality metrics. The alert classification system may enable healthcare providers to prioritize their review activities and focus attention on the most clinically significant issues identified through the enhanced medical analysis process.

With continued reference to FIG. 1, the alert generation may include specific recommendations and supporting clinical context that provide healthcare providers with actionable information for addressing identified inconsistencies. The alerts may include detailed explanations of the identified inconsistencies, references to the specific knowledge graph information that supports the alert generation, and suggested corrective actions such as diagnostic code revisions, additional laboratory testing, or documentation clarification. The recommendation system may provide healthcare providers with clear guidance for resolving identified issues while maintaining clinical autonomy in decision-making processes.

The step 170 may include checking for missing laboratory test results based on diagnosis codes and their relationships in the knowledge graph through systematic analysis of documented diagnoses and corresponding clinical evidence requirements. The missing laboratory test checking process may utilize the semantic relationships established through cross-coding mapping to determine which specific laboratory procedures are clinically associated with documented diagnoses. When a diagnostic code is mapped to a knowledge graph concept that includes relationships to laboratory procedures, the checking system may identify these laboratory tests as clinically relevant for verifying the accuracy and appropriateness of the documented diagnosis.

As further shown in FIG. 1, the checking for missing laboratory test results may involve systematic searching of laboratory information systems, electronic health record databases, and clinical data repositories to locate test results corresponding to the identified laboratory procedures. The checking system may query structured data sources using laboratory test codes, procedure identifiers, and test name variations to comprehensively search for relevant laboratory results. The structured data analysis may examine both recent test results and historical laboratory data to identify all available measurements related to the diagnostic validation requirements established through knowledge graph analysis.

The missing laboratory test detection may occur when the checking system identifies that specific laboratory tests should be present based on documented diagnoses, but no corresponding test results are found in the patient's structured laboratory data. The missing test detection process may generate alerts or recommendations indicating that specific laboratory measurements may be needed to support or verify the documented diagnostic classifications. Missing test identification may highlight potential gaps in the diagnostic workup that could affect the accuracy or completeness of the clinical documentation and may provide specific test recommendations based on the semantic relationships retrieved from knowledge graph systems.

Referring again to FIG. 1, the step 170 may include determining quality and accuracy of a patient medical file through comprehensive evaluation of documentation completeness, coding accuracy, and clinical consistency across multiple elements of the medical record. The quality determination process may analyze the enhanced medical analysis results to assess the overall reliability and completeness of medical documentation based on the consistency between documented clinical information and established medical knowledge retrieved from knowledge graph systems. The accuracy assessment may evaluate the specificity and appropriateness of diagnostic codes, the therapeutic appropriateness of medication prescriptions, and the presence of supporting clinical evidence for documented diagnoses.

The quality and accuracy determination may generate comprehensive assessment reports that summarize the overall documentation quality based on multiple analytical criteria including coding specificity, clinical consistency, and completeness of supporting evidence. The assessment reports may include quality scores that quantify the reliability of medical documentation based on the degree of agreement between documented clinical information and established medical knowledge. The quality determination process may identify areas for documentation improvement, highlight potential clinical issues requiring physician review, and provide recommendations for enhancing the accuracy and completeness of medical records.

As shown in FIG. 1, the step 170 may implement a real-time notification system that provides immediate alerts and recommendations to physicians during active patient encounters when potential issues are identified through automated analysis of medical documentation. The real-time notification system may operate continuously during clinical documentation processes to detect coding errors, diagnostic inconsistencies, missing laboratory tests, and other clinical discrepancies as they occur within the medical note creation workflow. The real-time nature of the notification system may enable physicians to address identified issues immediately during patient visits rather than discovering problems during subsequent documentation review processes.

The real-time notification system may continuously monitor the medical documentation analysis pipeline to identify situations that warrant immediate physician attention. The notification system may evaluate the results of language model analysis, knowledge graph validation, and cross-coding verification processes to detect various types of clinical issues including inaccurate diagnostic codes, missing laboratory test orders, contradictory clinical findings, and incomplete documentation elements. Each type of identified issue may trigger specific notification protocols designed to provide appropriate clinical context and recommended actions for resolution based on the enhanced medical analysis results generated through knowledge graph augmentation.

With continued reference to FIG. 1, the real-time notification system may provide multiple communication channels for presenting alerts to physicians during clinical encounters. Notification delivery may include visual alerts within electronic health record interfaces, pop-up messages during documentation entry processes, and integrated warnings within clinical decision support systems. The delivery system may be configured to present notifications at appropriate points in clinical workflows where physicians can effectively review and respond to the identified issues without disrupting patient care activities. The notification timing may be optimized to ensure that alerts are presented at clinically appropriate moments when physicians can effectively address identified issues while maintaining efficient clinical workflow patterns.

The real-time notification system may include interactive notification interfaces that enable physicians to respond to alerts directly within the notification system to acknowledge issues, request additional information, or initiate corrective actions. Interactive capabilities may include options to accept suggested diagnostic code changes, order recommended laboratory tests, or request detailed explanations of identified inconsistencies based on the enhanced medical analysis results. The interactive interface may streamline the process of addressing identified issues and provide feedback to the system about the accuracy and utility of generated notifications, enabling continuous improvement of notification algorithms and clinical relevance.

As further shown in FIG. 1, the step 170 may include notification acknowledgment tracking that monitors physician responses to alerts to ensure that identified issues receive appropriate clinical attention and to provide feedback for optimizing the notification system performance. Acknowledgment tracking may record which notifications are reviewed, which recommendations are accepted, and which alerts are dismissed without action. The tracking information may be used to refine notification algorithms, adjust alert prioritization criteria, and improve the clinical relevance of generated notifications based on physician feedback and response patterns observed during real-time clinical use.

The response operations within the step 170 may include alert suppression mechanisms that prevent redundant notifications for issues that have already been addressed or acknowledged by physicians during the current clinical encounter. The suppression system may track resolved issues and avoid generating duplicate alerts for the same clinical problems within specified time periods. Alert suppression may reduce notification fatigue and ensure that physicians receive focused alerts about new or unresolved issues rather than repeated warnings about previously addressed problems, thereby maintaining the effectiveness and clinical utility of the real-time notification system.

Referring to FIG. 1, the step 170 may provide customizable notification preferences that enable physicians and healthcare organizations to configure alert settings according to individual practice patterns, clinical specialties, and institutional requirements. Customization options may include notification frequency settings, alert priority thresholds, preferred delivery methods, and specialty-specific notification criteria. The preference system may allow physicians to tailor the notification experience to their specific clinical needs while maintaining appropriate safety and quality assurance standards based on the enhanced medical analysis results generated through knowledge graph augmentation processes.

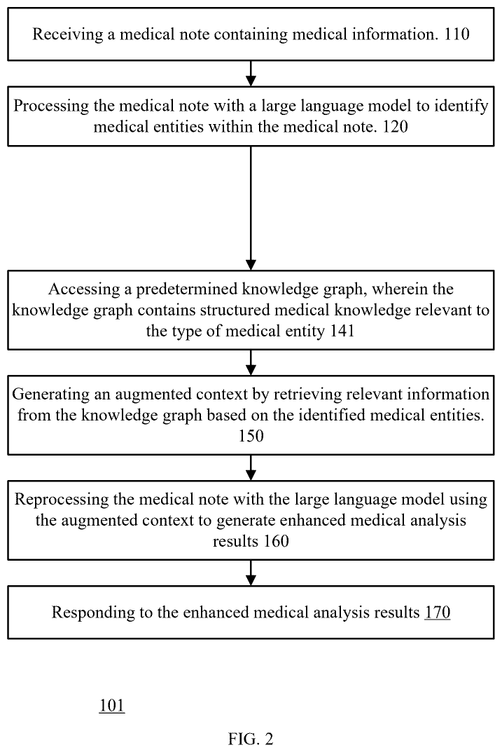

Referring to FIG. 2, a method 101 for analyzing medical notes using knowledge graph enhanced language models may provide a streamlined workflow where the knowledge graph to be accessed is predetermined, eliminating the need for knowledge graph selection based on entity type determination. The method 101 may offer a simplified processing approach that directly accesses a specified knowledge repository based on system configuration or clinical context requirements without requiring analysis of entity types or selection between multiple knowledge graph systems. The predetermined knowledge graph approach may reduce computational overhead and processing complexity while maintaining the analytical benefits of knowledge graph augmentation for medical documentation analysis operations.

The method 101 may be particularly suitable for clinical environments where a single comprehensive knowledge graph system is utilized for all medical documentation analysis operations, or where the type of medical documentation being processed consistently requires access to the same knowledge graph resources. Healthcare organizations that have standardized on specific medical coding systems or knowledge repositories may benefit from the streamlined processing approach provided by the method 101. The predetermined approach may enable optimized query formulation and execution based on the known structure and organization of the specific knowledge graph system being utilized, potentially improving processing efficiency and response times for routine medical documentation analysis tasks.

As shown in FIG. 2, the method 101 may begin with steps 110 that is followed by step 120.

Step 120 may be followed by the step 141 of accessing the predetermined knowledge graph, wherein the knowledge graph contains structured medical knowledge relevant to the type of medical entity. The predetermined knowledge graph access may utilize the extracted medical entities directly to formulate queries without requiring analysis of entity types or selection between multiple knowledge graph systems. The accessing operation may involve establishing connections to the predetermined knowledge graph system, formulating appropriate queries based on identified medical entities, and retrieving structured medical knowledge that provides contextual information to support enhanced language model analysis.

The predetermined knowledge graph may contain comprehensive medical knowledge spanning multiple entity types and clinical domains to support analysis of diverse medical documentation content. The knowledge graph may include diagnostic coding systems, medication databases, laboratory test classifications, and procedure codes organized in hierarchical structures or semantic relationship networks that enable systematic querying and retrieval of contextual information. The predetermined approach may enable optimized query formulation and execution based on the known structure and organization of the specific knowledge graph system, potentially reducing query processing time and improving overall system performance.

Referring again to FIG. 2, the step 141 may be followed by the step 150 that is followed by step 160 which in turn is followed by step 170.

Method 101 may provide computational efficiency benefits compared to approaches that require entity type determination and knowledge graph selection by eliminating the processing overhead associated with entity type analysis and knowledge repository selection operations. The predetermined approach may enable faster processing times and more predictable resource utilization patterns, making the method 101 suitable for high-volume clinical environments where consistent processing performance is required. The streamlined workflow may reduce system complexity while maintaining the analytical benefits of knowledge graph augmentation for medical documentation analysis.

As further shown in FIG. 2, the method 101 may be implemented using a computer-readable medium comprising instructions that, when executed by a processor, cause the processor to perform the method for analyzing medical notes using knowledge graph enhanced language models. The computer-readable medium may store executable instructions that implement the streamlined workflow of the method 101, including the predetermined knowledge graph access approach that eliminates entity type determination requirements. The instructions may configure the processor to perform the receiving, processing, accessing, generating, reprocessing, and responding operations in the predetermined sequence while utilizing the specified knowledge graph system for all medical documentation analysis operations.

The computer-readable medium implementation may provide deployment flexibility for healthcare organizations that prefer to utilize standardized knowledge graph systems or have established medical coding standards that align with specific knowledge repositories. The predetermined approach implemented through the computer-readable medium may enable consistent analytical outcomes and simplified system maintenance compared to more complex implementations that require management of multiple knowledge graph systems and entity type determination algorithms.

Referring to FIG. 2, the method 101 may be particularly suitable for clinical specialties that primarily utilize specific medical coding systems or knowledge repositories that align with the predetermined knowledge graph selection. Specialized clinical environments such as radiology departments that primarily use specific procedure coding systems, laboratory medicine practices that focus on particular test classifications, or specialty clinics that work with defined diagnostic categories may benefit from the streamlined approach provided by the method 101. The predetermined knowledge graph access may enable optimization of query patterns and knowledge retrieval operations for specific clinical domains while maintaining comprehensive analytical capabilities.

The method 101 may support batch processing operations where large volumes of similar medical documentation are analyzed using the same predetermined knowledge graph system. Batch processing scenarios may include retrospective analysis of medical records, quality assurance reviews of clinical documentation, or research applications that require consistent analytical approaches across large datasets. The predetermined approach may enable efficient processing of multiple documents through optimized knowledge graph access patterns and reduced computational overhead compared to approaches that require individual entity type determination for each document.

As shown in FIG. 2, the method 101 may provide simplified system configuration and maintenance requirements compared to more complex implementations that support multiple knowledge graph systems and dynamic entity type determination. Healthcare organizations may configure the method 101 by specifying the predetermined knowledge graph system and establishing appropriate access credentials and query optimization parameters. The simplified configuration approach may reduce implementation complexity and ongoing maintenance requirements while providing reliable medical documentation analysis capabilities that leverage structured medical knowledge for enhanced analytical accuracy and clinical relevance.

Referring to FIG. 2, the step 141 of accessing the predetermined knowledge graph may provide a streamlined approach for knowledge graph interaction that eliminates the computational overhead associated with entity type analysis and knowledge repository selection operations. The step 141 may utilize the extracted medical entities directly to formulate queries without requiring analysis of entity types or selection between multiple knowledge graph systems, thereby simplifying the workflow and reducing processing complexity while maintaining comprehensive access to structured medical knowledge. The predetermined knowledge graph access may enable optimized query formulation and execution based on the known structure and organization of the specific knowledge graph system being utilized.

As shown in FIG. 2, the step 141 may implement direct entity-to-query mapping processes that automatically translate extracted medical entities into appropriate knowledge graph queries without intermediate entity type classification steps. The direct mapping approach may utilize predefined query patterns that are specifically optimized for the predetermined knowledge graph system, enabling efficient access to relevant medical knowledge regardless of the specific entity types present in the medical documentation. The query formulation process may analyze the extracted entity values and automatically generate appropriate database queries, API requests, or knowledge graph traversal operations based on the known data structures and access methods of the predetermined knowledge graph system.

The predetermined knowledge graph access may support unified query interfaces that accommodate diverse medical entity types through comprehensive query patterns designed to retrieve relevant information for diagnoses, medications, laboratory results, and medical procedures from a single knowledge repository. The unified approach may eliminate the need for entity type-specific query strategies by utilizing query patterns that can effectively retrieve contextual information for multiple entity categories simultaneously. The predetermined knowledge graph may contain structured medical knowledge organized in hierarchical classifications, semantic relationship networks, or cross-referenced coding systems that enable comprehensive information retrieval through standardized query approaches.

With continued reference to FIG. 2, the step 141 may implement optimized query formulation processes that leverage the known structure and organization of the specific knowledge graph system to generate efficient database access operations. The optimization may involve pre-compiled query templates that are specifically designed for the predetermined knowledge graph architecture, enabling rapid query execution with minimal processing overhead. The query optimization may utilize indexing strategies, connection pooling, and result caching mechanisms that are tailored to the specific characteristics of the predetermined knowledge graph system, thereby improving query response times and overall system performance.

The predetermined approach may enable batch query operations that retrieve comprehensive medical knowledge for multiple extracted entities through coordinated database access operations. The batch processing capability may reduce the number of individual database queries required by combining multiple entity lookups into efficient bulk retrieval operations. As further shown in FIG. 2, the step 141 may utilize connection optimization techniques that maintain persistent connections to the predetermined knowledge graph system to eliminate connection establishment overhead during repeated query operations. The connection optimization may include connection pooling mechanisms that manage multiple concurrent database connections, load balancing strategies that distribute query operations across multiple database instances, and failover capabilities that ensure continued access to medical knowledge even when individual database components become unavailable.

The predetermined knowledge graph access may implement query result caching mechanisms that store frequently accessed medical knowledge in high-speed memory systems to reduce database access latency for common query patterns. The caching system may maintain hierarchical code structures, semantic relationship data, and cross-referencing information in memory to enable rapid retrieval during subsequent analysis operations. The cache management may include automatic cache invalidation processes that ensure cached medical knowledge remains current with updates to the underlying knowledge graph system.

Referring again to FIG. 2, the step 141 may support simple mode operation where an action for accessing the knowledge graph is predetermined based on the type of medical entity identified, even though explicit entity type determination is not performed. The simple mode may utilize pattern recognition algorithms that automatically identify appropriate query strategies based on the characteristics of extracted medical entities without requiring formal entity type classification processes. The predetermined action selection may analyze entity attributes such as terminology patterns, numerical value presence, or contextual indicators to determine appropriate knowledge graph access methods.

The simple mode operation may implement rule-based query selection processes that automatically choose appropriate query templates based on entity characteristics detected during the extraction phase. The rule-based approach may utilize decision trees or pattern matching algorithms that map entity features to specific query strategies without requiring explicit entity type labels. The predetermined action selection may enable consistent query behavior while maintaining the computational efficiency benefits of the predetermined knowledge graph access approach.

As shown in FIG. 2, the step 141 may provide computer-readable medium implementation support where instructions stored on the medium cause a processor to perform the predetermined knowledge graph access operations. The computer-readable medium may contain executable code that implements the optimized query formulation processes, connection management mechanisms, and result caching strategies specifically designed for the predetermined knowledge graph system. The instructions may configure the processor to establish persistent connections to the knowledge graph, execute optimized query patterns, and retrieve comprehensive medical knowledge without requiring entity type determination or knowledge repository selection operations.

The computer-readable medium implementation may include configuration parameters that specify the predetermined knowledge graph system characteristics, access credentials, and query optimization settings. The stored instructions may implement error handling mechanisms that provide graceful degradation when the predetermined knowledge graph system becomes temporarily unavailable, including fallback query strategies and alternative data access methods that maintain system functionality during knowledge graph maintenance or update operations.

With continued reference to FIG. 2, the step 141 may enable scalable knowledge graph access that accommodates varying processing loads through dynamic resource allocation and query optimization strategies. The scalable access may support concurrent query operations from multiple analysis processes while maintaining consistent response times and data integrity. The predetermined knowledge graph access may implement load monitoring mechanisms that automatically adjust query execution strategies based on system resource availability and processing demand patterns.

The step 141 may provide comprehensive logging and monitoring capabilities that track query performance, resource utilization, and access patterns to support system optimization and troubleshooting operations. The monitoring system may collect metrics on query execution times, cache hit rates, and connection utilization to enable continuous improvement of the predetermined knowledge graph access performance. The logging capabilities may provide detailed audit trails of knowledge graph access operations to support compliance requirements and system analysis activities.

Referring to FIG. 2, a computer-readable medium may comprise instructions that, when executed by a processor, cause the processor to perform a method for analyzing medical notes using knowledge graph enhanced language models. The computer-readable medium may store executable instructions that implement the analytical capabilities described in the method 101 through software-based processing operations that coordinate natural language processing components with knowledge graph access systems. The instructions may configure the processor to perform systematic medical documentation analysis operations that combine language model capabilities with structured medical knowledge retrieval to generate enhanced analytical results.

The computer-readable medium implementation may include instructions for receiving a medical note containing medical information through software-based data ingestion processes that accommodate diverse medical document formats and sources. The receiving instructions may configure the processor to establish data interfaces that accept structured electronic health record data, unstructured text documents, and standardized medical messaging formats through programmatic access methods. The software implementation may include document validation routines that verify data integrity and completeness, format standardization algorithms that convert diverse document formats into consistent data structures, and content preparation processes that organize medical information for subsequent language model analysis operations.

As shown in FIG. 2, the instructions may implement receiving operations through application programming interfaces that enable direct integration with electronic health record systems, file processing routines for batch analysis of medical documents, and streaming data interfaces for real-time processing of medical documentation as documents are created within healthcare information systems. The software-based receiving implementation may include error handling mechanisms that manage document format inconsistencies, encoding validation processes that ensure proper character representation, and content segmentation algorithms that organize document sections into logical components suitable for natural language processing operations.

The computer-readable medium may contain instructions for processing the medical note with a large language model to identify medical entities within the medical note through software-based natural language processing operations that utilize transformer-based architectures or other neural network approaches. The processing instructions may configure the processor to load language model parameters, initialize medical terminology recognition modules, and execute entity extraction algorithms that systematically identify and classify medical entities within clinical text. The software implementation may include named entity recognition routines that classify different portions of medical text according to predefined medical entity categories and confidence scoring mechanisms that assess the reliability of extraction and classification operations.

With continued reference to FIG. 2, the processing instructions may implement medical entity extraction through pattern matching algorithms that identify medical terminology based on lexical patterns, contextual analysis routines that consider surrounding text to disambiguate medical terms, and classification algorithms that assign entity labels based on medical terminology patterns and contextual indicators. The software-based processing may include entity validation routines that verify extraction accuracy, filtering mechanisms that remove low-confidence extractions, and output formatting processes that structure extracted medical information for subsequent knowledge graph access operations.

The instructions may implement language model processing through software modules that manage model initialization, input preprocessing, inference execution, and output postprocessing operations. The software implementation may include memory management routines that efficiently load and maintain language model parameters, batch processing capabilities that enable concurrent analysis of multiple medical documents, and optimization algorithms that improve processing performance through caching mechanisms and parallel execution strategies. The processing instructions may configure the processor to utilize specialized hardware acceleration when available, including graphics processing units or tensor processing units that provide enhanced performance for neural network inference operations.

As further shown in FIG. 2, the computer-readable medium may include instructions for determining a type of medical entity from the identified medical entities through software-based classification algorithms that analyze entity characteristics and contextual indicators. The determining instructions may configure the processor to execute classification routines that evaluate lexical patterns, semantic features, and contextual relationships to categorize extracted entities according to standardized medical entity types. The software implementation may include decision tree algorithms that map entity features to specific entity type classifications, pattern recognition routines that identify characteristic terminology patterns, and confidence assessment mechanisms that evaluate the reliability of entity type determinations.

The determining instructions may implement entity type classification through machine learning algorithms that have been trained on annotated medical documentation datasets to recognize patterns associated with different medical entity categories. The software-based classification may include feature extraction routines that identify relevant textual and contextual characteristics, classification model execution processes that apply trained algorithms to extracted entities, and result validation mechanisms that verify classification accuracy. The determining implementation may include multi-class classification capabilities that handle entities containing multiple medical entity types and disambiguation algorithms that resolve ambiguous entity classifications based on contextual analysis.

Referring again to FIG. 2, the instructions may implement entity type determination through rule-based classification systems that utilize predefined criteria for distinguishing between diagnostic entities, medication entities, laboratory result entities, and procedure entities. The rule-based approach may include conditional logic that evaluates entity characteristics against established patterns, scoring mechanisms that assess the strength of classification evidence, and threshold-based decision processes that assign entity type labels based on classification confidence levels. The software implementation may include exception handling routines that manage ambiguous or uncertain entity classifications and logging mechanisms that track classification performance for system optimization purposes.

The computer-readable medium may contain instructions for generating an augmented context by retrieving relevant information from the knowledge graph based on the identified medical entities through software-based knowledge graph access and information retrieval operations. The augmented context generation instructions may configure the processor to formulate knowledge graph queries based on extracted medical entities, execute database access operations to retrieve structured medical knowledge, and format retrieved information into contextual additions suitable for language model processing. The software implementation may include query optimization routines that improve database access performance, result processing algorithms that organize retrieved knowledge according to clinical relevance, and context formatting processes that structure augmented information for effective integration with original medical documentation.

As shown in FIG. 2, the generating instructions may implement knowledge graph access through database connectivity modules that establish connections to knowledge graph systems, query formulation algorithms that translate medical entities into appropriate database queries, and result retrieval processes that obtain comprehensive medical knowledge from structured repositories. The software-based implementation may include connection pooling mechanisms that manage database connections efficiently, transaction management routines that ensure data consistency during complex query operations, and caching systems that store frequently accessed medical knowledge to reduce database access latency.

The augmented context generation instructions may implement hierarchical expansion algorithms that retrieve comprehensive code trees and classification structures surrounding initially identified medical entities. The software implementation may include tree traversal routines that navigate hierarchical knowledge structures, relationship exploration algorithms that identify semantic connections between medical concepts, and cross-referencing processes that map entities between different medical coding systems. The generating instructions may configure the processor to execute parallel query operations that retrieve information from multiple knowledge sources simultaneously and result in aggregation algorithms that combine retrieved knowledge into coherent contextual datasets.

With continued reference to FIG. 2, the instructions may implement augmented context generation through template-based query systems that utilize predefined query patterns optimized for specific knowledge graph architectures. The template-based implementation may include query template libraries that contain optimized patterns for common medical entity types, parameter substitution routines that populate templates with extracted entity values, and query execution engines that process populated templates against knowledge graph systems. The software-based generating operations may include result validation mechanisms that verify retrieved information accuracy and relevance filtering algorithms that prioritize pertinent knowledge based on clinical context.

The computer-readable medium implementation may include instructions for coordinating the execution sequence of receiving, processing, determining, and generating operations through workflow management software that ensures proper data flow and processing dependencies. The coordination instructions may configure the processor to manage processing state information across multiple analysis steps, handle error conditions that may occur during any processing phase, and provide progress monitoring capabilities that track analysis completion status. The software implementation may include parallel processing capabilities that enable concurrent execution of independent analysis operations and synchronization mechanisms that coordinate data sharing between different processing components.

As further shown in FIG. 2, the instructions may implement comprehensive error handling and recovery mechanisms that provide robust operation when individual software components encounter processing errors or unexpected conditions. The error handling implementation may include exception management routines that capture and process error conditions, fallback processing strategies that provide alternative analysis approaches when primary methods fail, and logging systems that record error information for troubleshooting and system improvement purposes. The software-based error handling may include automatic retry mechanisms for transient failures and graceful degradation capabilities that maintain partial functionality when complete analysis operations cannot be completed.

The computer-readable medium may contain instructions for implementing performance optimization features that improve processing efficiency and resource utilization during medical documentation analysis operations. The optimization instructions may configure the processor to utilize memory management strategies that efficiently allocate and deallocate processing resources, implement caching mechanisms that reduce redundant processing operations, and execute load balancing algorithms that distribute processing tasks across available computational resources. The software implementation may include performance monitoring routines that track processing times and resource utilization patterns to enable continuous system optimization and capacity planning activities.

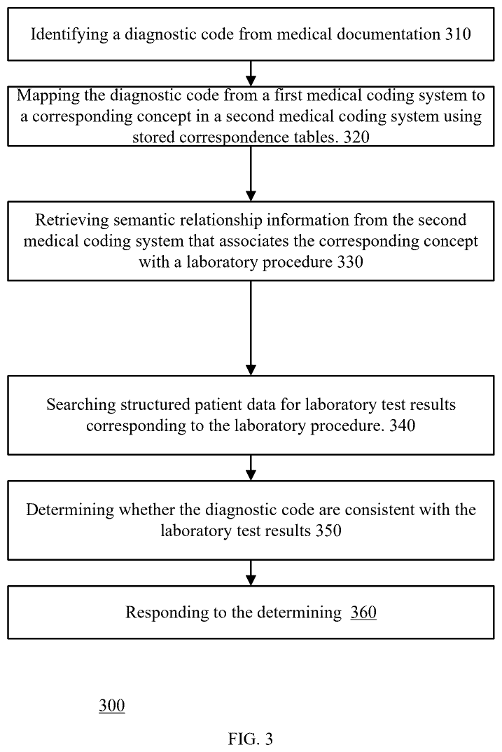

Referring to FIG. 3, any reference to laboratory test results should be applied mutatis mutandis to medications, procedures, and disease activity score. It should be noted that, can also be used to find potential inconsistencies.

Method 300 is for validating medical diagnoses using cross-coding system mapping may provide systematic procedures for verifying documented diagnoses by analyzing structured patient data to identify corresponding laboratory test results and detect missing or contradictory clinical findings. The method 300 may establish relationships and perform translations between different medical coding standards and classification systems used in healthcare environments to enable comprehensive medical documentation validation that leverages semantic relationships available in comprehensive medical terminology systems while maintaining compatibility with standard diagnostic coding systems used for billing and administrative purposes.

The method 300 may utilize cross-coding system mapping functionality that establishes relationships and performs translations between different medical coding standards such as ICD-10 Clinical Modification and SNOMED CT mapping operations. The cross-coding mapping approach may enable the validation process to transition from diagnostic coding systems optimized for billing purposes to more semantically rich terminology systems that include detailed clinical relationships and procedural associations. The mapping functionality may access predefined correspondence tables that associate codes from one medical classification system with semantically equivalent or related codes in other systems, along with relationship type indicators that specify the nature of the correspondence between different coding frameworks.

As shown in FIG. 3, the method 300 may begin with the step 310 of identifying a diagnostic code from medical documentation through systematic extraction of diagnostic codes that have been assigned to patient conditions within medical notes, billing records, or electronic health record systems. The identification operation may extract diagnostic codes represented in various medical coding systems including ICD-10 Clinical Modification, ICD-11, or other diagnostic classification frameworks through parsing of structured data fields, extraction of codes from unstructured text, or access to diagnostic code databases associated with patient records. The diagnostic code identification may provide the foundation for subsequent cross-coding mapping operations that enable access to semantic relationships and clinical criteria stored in alternative medical terminology systems.

With continued reference to FIG. 3, the step 310 may be followed by the step 320 of mapping the diagnostic code from a first medical coding system to a corresponding concept in a second medical coding system using stored correspondence tables. The mapping operation may access predefined correspondence tables that associate codes from one medical classification system with semantically equivalent or related codes in other systems through systematic cross-referencing operations. The correspondence tables may include metadata that describes the nature of the relationship between codes from different systems, such as exact matches, approximate equivalents, or hierarchical relationships that enable the validation process to leverage the semantic richness of the target coding system while maintaining compatibility with the source diagnostic coding framework.

The cross-coding mapping functionality may support ICD-10 Clinical Modification to SNOMED CT mapping operations that represent common cross-coding scenarios where diagnostic codes from the International Classification of Diseases system are mapped to corresponding concepts in the Systematized Nomenclature of Medicine Clinical Terms. The ICD-10-CM to SNOMED CT mapping may enable the validation process to access the semantic richness of SNOMED CT while maintaining compatibility with ICD-10-CM codes commonly used for billing and administrative purposes in healthcare systems. The mapping correspondence may associate ICD-10-CM diagnostic codes with semantically equivalent or related SNOMED CT concept identifiers that provide additional semantic information and relationship details not available within the ICD-10-CM system alone.

As further shown in FIG. 3, the step 320 may be followed by the step 330 of retrieving semantic relationship information from the second medical coding system that associates the corresponding concept with a laboratory procedure. The semantic relationship retrieval may access the knowledge graph structure of the second medical coding system to identify relationships between the mapped diagnostic concept and related clinical entities such as laboratory procedures, anatomical structures, and clinical findings. The retrieved relationship information may include specific relationship types such as “Interprets” relationships that connect diagnostic concepts to laboratory tests that provide clinical evidence for the diagnostic classification, enabling the validation process to identify which laboratory tests should be present in patient records to support or verify the documented diagnosis.

The semantic relationship information retrieval may utilize the mapped SNOMED CT concept identifier to access the rich relationship structure available within the SNOMED CT terminology system. The SNOMED CT concept for specific diagnostic conditions may include various relationship types that connect the diagnostic concept to related clinical entities through semantic associations that provide additional analytical capabilities extending beyond the hierarchical structure available within the ICD-10-CM system. The semantic relationships may include “Interprets” relationships that connect diagnostic concepts to specific laboratory procedures, indicating the laboratory tests that provide clinical evidence for the diagnostic classification and enabling systematic identification of expected clinical evidence for documented diagnoses.

Referring again to FIG. 3, the step 330 may be followed by the step 340 of searching structured patient data for laboratory test results corresponding to the laboratory procedure. The searching operation may query laboratory information systems, electronic health record databases, and clinical data repositories to locate test results corresponding to the identified laboratory procedures through systematic database access operations. The search may utilize laboratory test codes, procedure identifiers, and test name variations to comprehensively search for relevant laboratory results across multiple data sources and time periods. The structured data analysis may examine both recent test results and historical laboratory data to identify all available measurements related to the diagnostic validation requirements established through the semantic relationship analysis.

The searching operation may implement comprehensive data access strategies that accommodate variations in laboratory test naming conventions, coding systems, and data storage formats used by different laboratory information systems and healthcare organizations. The search algorithms may utilize multiple laboratory test identifiers including LOINC codes, laboratory-specific test codes, and text-based test name variations to ensure comprehensive identification of relevant laboratory results. The searching process may include temporal analysis capabilities that examine laboratory test timing relationships and result trend analysis to provide comprehensive assessment of available clinical evidence for documented diagnoses.

As shown in FIG. 3, the step 340 may be followed by the step 350 of determining whether the diagnostic code is consistent with the laboratory test results through systematic analysis of retrieved laboratory test values to evaluate their consistency with the documented diagnostic classification. The determination operation may analyze the retrieved laboratory test values against established reference ranges and clinical thresholds to classify the results as supporting or contradicting the documented diagnosis. The consistency evaluation may compare laboratory test results with diagnostic criteria and clinical guidelines retrieved through the cross-coding mapping process to identify situations where laboratory results support the documented diagnosis, contradict the diagnostic classification, or provide insufficient evidence for diagnostic verification.

The determination process may implement sophisticated analytical algorithms that consider multiple factors including laboratory reference ranges, patient demographics, clinical context, and temporal relationships between test results and diagnostic documentation. The consistency analysis may account for institution-specific reference ranges, age-adjusted normal values, and gender-specific thresholds when evaluating laboratory test results against documented diagnoses. The determination operation may generate confidence scores that indicate the strength of agreement between laboratory evidence and documented diagnoses based on the clinical significance of specific laboratory tests and the degree of deviation from expected values.

With continued reference to FIG. 3, the step 350 may be followed by the step 360 of responding to the determining operation through generation of validation results that indicate consistency between the diagnostic code and the laboratory test results. The responding operation may create structured outputs that present the validation findings in formats suitable for clinical review and decision-making, including summaries of identified laboratory tests, consistency assessments, and detailed explanations of any identified discrepancies. The validation result generation may include comprehensive assessment reports that summarize the overall diagnostic validation based on multiple analytical criteria including laboratory evidence strength, clinical consistency, and completeness of supporting documentation.

The responding operation may include generating alerts when inconsistency is found between documented diagnoses and available laboratory evidence through systematic identification of situations that warrant clinical attention and possible diagnostic review. The alert generation may create notifications classified according to clinical urgency that include specific recommendations for diagnostic review, additional testing, or documentation clarification to resolve identified discrepancies. The inconsistency alerts may provide detailed explanations of the identified contradictions, references to the specific laboratory values and reference ranges that contribute to the inconsistency determination, and suggested corrective actions based on the clinical evidence and diagnostic criteria accessed through the cross-coding mapping process.

As further shown in FIG. 3, the method 300 may provide technical benefits including improved diagnostic accuracy through systematic verification of documented diagnoses against objective laboratory evidence retrieved through cross-coding system mapping. The cross-coding mapping functionality may enable access to semantic relationships and clinical criteria that would not be available through single coding system analysis, thereby providing more comprehensive diagnostic validation capabilities. The method 300 may reduce diagnostic errors by identifying situations where documented diagnoses lack supporting laboratory evidence or where laboratory results contradict documented diagnostic classifications, enabling healthcare providers to address potential diagnostic issues before they affect patient care decisions.

The cross-coding system mapping approach may enable interoperability between different medical coding systems used in healthcare environments by providing systematic translation capabilities that maintain semantic accuracy while accommodating diverse coding standards and terminology systems. The method 300 may support healthcare organizations that utilize multiple coding systems by providing unified validation capabilities that leverage the strengths of different medical terminology frameworks. The systematic validation approach may improve documentation quality by identifying missing clinical evidence, inconsistent diagnostic classifications, and potential coding errors that could affect billing accuracy, clinical quality metrics, and patient care outcomes.

Referring to FIG. 3, the step 310 of identifying a diagnostic code from medical documentation may involve comprehensive extraction processes that systematically locate and retrieve diagnostic codes that have been assigned to patient conditions within various types of medical records and healthcare information systems. The identification operation may analyze structured data fields within electronic health record systems where diagnostic codes are stored in designated database fields, extract codes embedded within unstructured clinical text through pattern recognition algorithms, and access diagnostic code databases that maintain associations between patient records and assigned diagnostic classifications. The diagnostic code identification may accommodate various medical coding systems including ICD-10 Clinical Modification, ICD-11, SNOMED CT, and other diagnostic classification frameworks that may be utilized within different healthcare environments and clinical specialties.

The step 310 may implement sophisticated parsing algorithms that can identify diagnostic codes regardless of their presentation format within medical documentation. The identification process may recognize diagnostic codes presented as standalone alphanumeric identifiers, codes embedded within clinical narrative text, and codes associated with billing or administrative data structures. The parsing algorithms may utilize regular expression patterns that match standard diagnostic code formats, contextual analysis that identifies diagnostic code references within clinical text, and database query operations that retrieve codes from structured medical record systems. The identification operation may include validation mechanisms that verify the format and validity of extracted diagnostic codes against established coding system standards and reference databases.

As shown in FIG. 3, the diagnostic code identification may support multiple extraction scenarios including real-time analysis of newly created medical documentation, batch processing of historical medical records, and integration with existing healthcare information systems that maintain diagnostic code databases. The identification process may accommodate variations in diagnostic code presentation including codes with or without decimal separators, codes presented with descriptive text, and codes stored in different database field formats. The extraction algorithms may implement fuzzy matching capabilities that can identify diagnostic codes even when they contain minor formatting variations or typographical errors that might occur during manual data entry processes.

The step 310 may include confidence scoring mechanisms that assess the reliability of diagnostic code identification based on the clarity of code presentation, consistency with established coding formats, and agreement between multiple identification methods. The confidence scoring may enable subsequent processing operations to prioritize high-confidence code identifications and flag uncertain extractions for manual review or additional validation. The identification process may generate structured outputs that include the extracted diagnostic codes, their source locations within the medical documentation, confidence scores, and any associated descriptive text or clinical context that may support subsequent cross-coding mapping operations.

With continued reference to FIG. 3, the step 320 of mapping the diagnostic code from a first medical coding system to a corresponding concept in a second medical coding system using stored correspondence tables may provide systematic translation capabilities that enable access to semantic relationships and clinical criteria available in alternative medical terminology systems. The mapping operation may access predefined correspondence tables that have been established through expert analysis, algorithmic matching processes, and official mapping releases from medical coding organizations to ensure accurate and clinically appropriate translations between different coding frameworks. The correspondence tables may be maintained as structured databases that associate source codes with target codes along with relationship metadata that describes the nature and reliability of each mapping correspondence.

The mapping operation may utilize the extracted diagnostic codes from the step 310 as input parameters for database queries that search the stored correspondence tables for matching entries. The query process may implement exact matching algorithms that identify direct correspondences between codes from different systems, as well as approximate matching capabilities that can identify related concepts when exact equivalents are not available. The mapping queries may consider hierarchical relationships within coding systems to identify broader or narrower concepts when direct mappings are not established, enabling comprehensive cross-system translation even for specialized or rarely used diagnostic codes.

As further shown in FIG. 3, the stored correspondence tables may contain comprehensive mapping relationships that have been established through systematic analysis of medical coding systems by clinical experts, coding specialists, and medical terminology organizations. The correspondence tables may include mappings between ICD-10 Clinical Modification and SNOMED CT systems, relationships between different versions of ICD coding systems, and cross-references between national and international coding standards. The tables may be regularly updated to reflect changes in coding systems, new code releases, and refinements in mapping accuracy based on clinical feedback and usage patterns observed in healthcare environments.

The correspondence tables may organize mapping relationships using structured data formats that include source code identifiers, target code identifiers, relationship type indicators, and mapping confidence scores. The source code identifiers may specify the diagnostic codes from the first medical coding system that serve as the starting point for cross-system translation. The target code identifiers may specify the corresponding concepts in the second medical coding system that provide semantically equivalent or related diagnostic classifications. The relationship type indicators may describe the nature of the correspondence between source and target codes, enabling appropriate interpretation and utilization of mapping results.

Referring again to FIG. 3, the metadata that describes the nature of relationships between codes may include relationship type classifications that specify whether the mapping represents an exact equivalent, approximate equivalent, broader concept, narrower concept, or related concept correspondence. Exact equivalent relationships may indicate that the source and target codes represent essentially the same diagnostic concept with comparable clinical meaning and specificity. Approximate equivalent relationships may indicate that the codes represent similar but not identical diagnostic concepts that may have minor differences in scope, specificity, or clinical interpretation. Broader concept relationships may indicate that the target code represents a more general diagnostic category that encompasses the source code along with related conditions.

The metadata may include narrower concept relationships that indicate the target code represents a more specific diagnostic classification that falls within the broader category represented by the source code. Related concept relationships may indicate that the source and target codes represent diagnostically related conditions that share clinical characteristics, anatomical locations, or pathophysiological mechanisms without being direct equivalents. The relationship metadata may enable appropriate interpretation of mapping results and inform subsequent processing operations about the reliability and clinical appropriateness of cross-system translations.

As shown in FIG. 3, the metadata may include mapping confidence scores that quantify the reliability and accuracy of the correspondence between codes from different medical classification systems. The confidence scores may be based on expert validation processes, algorithmic similarity assessments, and clinical outcome analysis that evaluates the appropriateness of mapping relationships in real-world healthcare applications. High confidence scores may indicate mappings that have been extensively validated and demonstrate consistent clinical utility, while lower confidence scores may indicate mappings that require additional verification or may have limited applicability in certain clinical contexts.

The metadata may include temporal information that indicates when mapping relationships were established, last updated, or validated to ensure that cross-system translations reflect current coding standards and clinical practices. The temporal metadata may enable the mapping system to prioritize recently validated mappings and identify relationships that may require review or updating based on changes in coding systems or clinical guidelines. The correspondence tables may include versioning information that tracks changes in mapping relationships over time and enables historical analysis of diagnostic code translations for research or quality assurance purposes.

With continued reference to FIG. 3, the mapping operation may implement sophisticated query algorithms that can handle complex mapping scenarios including one-to-many relationships where a single source code corresponds to multiple target codes, many-to-one relationships where multiple source codes map to a single target code, and many-to-many relationships where complex diagnostic concepts require multiple code correspondences. The query algorithms may utilize ranking mechanisms that prioritize mapping results based on confidence scores, clinical relevance, and specificity to ensure that the most appropriate target codes are selected for subsequent processing operations.

The correspondence tables may be optimized for rapid query execution through indexing strategies that enable efficient lookup of mapping relationships during real-time diagnostic validation operations. The optimization may include database indexing on source code identifiers, target code identifiers, and relationship metadata to minimize query response times. The mapping system may implement caching mechanisms that store frequently accessed mapping relationships in high-speed memory to reduce database access latency during high-volume processing operations. The performance optimization may ensure that cross-system mapping operations do not introduce significant delays in diagnostic validation workflows while maintaining comprehensive access to mapping relationships across diverse medical coding systems.

Referring to FIG. 3, the step 330 of retrieving semantic relationship information from the second medical coding system that associates the corresponding concept with a laboratory procedure may provide systematic access to the knowledge graph structure of the target medical coding system to identify relationships between the mapped diagnostic concept and related clinical entities. The semantic relationship retrieval may utilize the mapped diagnostic concept identifier obtained through the step 320 to access the rich relationship structure available within comprehensive medical terminology systems such as SNOMED CT that organize medical concepts using multiple relationship types including hierarchical classifications, part-whole relationships, and clinical associations between different medical entities.

The step 330 may implement knowledge graph traversal algorithms that systematically explore the semantic relationship network surrounding the mapped diagnostic concept to identify connections to laboratory procedures, anatomical structures, clinical findings, and other medically relevant entities. The traversal algorithms may utilize graph database query languages such as Cypher or specialized knowledge graph access methods to navigate the relationship structure and retrieve comprehensive relationship information associated with the target diagnostic concept. The semantic relationship exploration may examine direct relationships that connect the diagnostic concept to immediately related entities, as well as multi-hop relationships that identify more distant but clinically relevant associations through intermediate concept nodes.

As shown in FIG. 3, the semantic relationship retrieval may identify specific relationship types such as “Interprets” relationships that connect diagnostic concepts to laboratory tests that provide clinical evidence for the diagnostic classification. The “Interprets” relationship type may represent a semantic association indicating that specific laboratory procedures provide clinical evidence or diagnostic support for particular medical conditions, enabling the validation process to identify which laboratory tests should be present in patient records to support or verify the documented diagnosis. The relationship identification may utilize relationship type filtering algorithms that focus on clinically relevant relationship categories while excluding administrative or purely taxonomic relationships that may not contribute to diagnostic validation objectives.

The step 330 may access semantic relationship databases that store comprehensive relationship information using structured data formats that include relationship identifiers, source concept identifiers, target concept identifiers, relationship type classifications, and relationship strength indicators. The relationship databases may organize semantic associations using standardized relationship vocabularies that provide consistent interpretation of relationship meanings across different medical domains and clinical specialties. The semantic relationship storage may utilize graph database architectures that enable efficient traversal of complex relationship networks and support sophisticated query operations that can explore multiple relationship types simultaneously.

With continued reference to FIG. 3, the semantic relationship retrieval may implement relationship validation mechanisms that verify the clinical appropriateness and current validity of identified relationships based on medical literature, clinical guidelines, and expert consensus. The validation processes may utilize relationship confidence scores that indicate the strength of evidence supporting specific semantic associations, enabling the retrieval system to prioritize well-established relationships while flagging uncertain or controversial associations for additional review. The relationship validation may include temporal analysis that considers the currency of relationship information and identifies relationships that may require updating based on evolving clinical knowledge or changes in diagnostic practices.

The step 330 may retrieve laboratory procedure associations through systematic analysis of “Interprets” relationships that connect the mapped diagnostic concept to specific laboratory test procedures, measurement techniques, or clinical assessment methods. The laboratory procedure identification may access comprehensive procedure databases that contain detailed information about laboratory tests including test names, procedure codes, measurement parameters, reference ranges, and clinical interpretation guidelines. The procedure association retrieval may utilize multiple identifier systems including LOINC codes, CPT procedure codes, and laboratory-specific test identifiers to ensure comprehensive identification of relevant laboratory procedures regardless of the coding systems used by different healthcare organizations.

As further shown in FIG. 3, the semantic relationship information retrieval may include relationship context analysis that examines the clinical significance and diagnostic relevance of identified laboratory procedure associations. The context analysis may evaluate the strength of diagnostic evidence provided by specific laboratory tests, the frequency of test utilization for particular diagnostic conditions, and the clinical guidelines that recommend specific laboratory assessments for documented diagnoses. The relationship context may include information about test sensitivity and specificity for particular diagnostic conditions, enabling the validation process to appropriately weight laboratory evidence based on the diagnostic utility of specific tests.

The step 330 may implement comprehensive relationship exploration algorithms that identify multiple laboratory procedures associated with a single diagnostic concept through various relationship pathways and semantic associations. The comprehensive exploration may identify primary laboratory tests that provide direct diagnostic evidence, secondary tests that support differential diagnosis processes, and monitoring tests that track disease progression or treatment response. The multi-procedure identification may enable comprehensive diagnostic validation that considers the full spectrum of laboratory evidence that may be relevant for specific medical conditions.

Referring again to FIG. 3, the semantic relationship retrieval may access relationship metadata that provides additional context about the nature and clinical significance of identified laboratory procedure associations. The relationship metadata may include information about the diagnostic phase during which specific laboratory tests are typically performed, such as initial diagnostic workup, confirmatory testing, or ongoing monitoring activities. The metadata may include clinical specialty associations that indicate which medical specialties typically order specific laboratory tests for particular diagnostic conditions, enabling the validation process to consider specialty-specific practice patterns and testing preferences.

The step 330 may utilize relationship inheritance mechanisms that identify laboratory procedure associations through hierarchical concept relationships within the semantic knowledge graph structure. The inheritance mechanisms may identify laboratory tests associated with broader diagnostic categories that encompass the specific mapped diagnostic concept, as well as tests associated with more specific diagnostic subcategories that fall within the mapped concept scope. The hierarchical relationship exploration may enable comprehensive identification of relevant laboratory procedures even when direct semantic associations are not explicitly established for specific diagnostic codes.

As shown in FIG. 3, the semantic relationship retrieval may implement relationship filtering algorithms that prioritize clinically relevant laboratory procedure associations based on diagnostic utility, clinical guidelines, and evidence-based medicine principles. The filtering algorithms may exclude laboratory procedures that have limited diagnostic value for specific conditions, tests that are primarily used for research purposes, or procedures that are not commonly available in routine clinical practice. The relationship filtering may utilize clinical decision support databases that contain evidence-based recommendations for laboratory testing in specific diagnostic scenarios.

The step 330 may provide relationship result formatting processes that organize the retrieved semantic relationship information into structured data formats suitable for subsequent processing operations within the method 300. The formatting processes may create standardized relationship records that include diagnostic concept identifiers, laboratory procedure identifiers, relationship type classifications, and clinical context information. The structured formatting may enable efficient processing of relationship information during subsequent steps of the diagnostic validation workflow while maintaining comprehensive access to the semantic richness of the underlying knowledge graph structure.

With continued reference to FIG. 3, the semantic relationship retrieval may include relationship caching mechanisms that store frequently accessed relationship information in high-speed memory systems to reduce knowledge graph access latency during repeated validation operations. The caching mechanisms may maintain relationship data for commonly encountered diagnostic concepts, enabling rapid retrieval of laboratory procedure associations without requiring repeated knowledge graph queries. The relationship caching may include cache invalidation processes that ensure cached relationship information remains current with updates to the underlying semantic knowledge graph systems.

The step 330 may implement relationship quality assessment processes that evaluate the completeness and accuracy of retrieved semantic relationship information based on clinical validation studies, expert review processes, and real-world usage patterns observed in healthcare environments. The quality assessment may generate relationship reliability scores that indicate the confidence level associated with specific laboratory procedure associations, enabling subsequent processing operations to appropriately weight relationship information based on its clinical validation status. The relationship quality evaluation may include feedback mechanisms that incorporate clinical user input and validation outcomes to continuously improve the accuracy and clinical utility of semantic relationship retrieval operations.

Referring to FIG. 3, the step 340 of searching structured patient data for laboratory test results corresponding to the laboratory procedure may implement comprehensive data access strategies that systematically query laboratory information systems, electronic health record databases, and clinical data repositories to locate test results corresponding to the laboratory procedures identified through the semantic relationship analysis. The searching operation may utilize the laboratory procedure identifiers retrieved during the step 330 to formulate database queries that comprehensively search for relevant laboratory results across multiple data sources and time periods. The structured data analysis may accommodate variations in laboratory test naming conventions, coding systems, and data storage formats used by different laboratory information systems and healthcare organizations to ensure comprehensive identification of relevant clinical evidence.

The searching operation may implement multi-system query algorithms that access diverse clinical data repositories including laboratory information management systems, electronic health record databases, clinical data warehouses, and specialized diagnostic testing systems. The query algorithms may utilize multiple laboratory test identifiers including LOINC codes for standardized laboratory test identification, CPT procedure codes for billing and administrative purposes, laboratory-specific test codes used by individual healthcare institutions, and text-based test name variations that may be employed in different clinical documentation systems. The comprehensive identifier utilization may ensure that relevant laboratory results are identified regardless of the specific coding systems or naming conventions used by different healthcare organizations or laboratory service providers.

As shown in FIG. 3, the step 340 may implement temporal analysis capabilities that examine laboratory test timing relationships and result trend analysis to provide comprehensive assessment of available clinical evidence for documented diagnoses. The temporal analysis may search for laboratory results within clinically relevant time periods surrounding the diagnostic documentation, including recent test results that may provide current clinical evidence and historical laboratory data that may establish baseline values or demonstrate disease progression patterns. The searching algorithms may utilize configurable time windows that can be adjusted based on the specific diagnostic condition, laboratory test characteristics, and clinical guidelines that specify appropriate testing intervals for particular medical conditions.

The structured patient data searching may include sophisticated pattern matching algorithms that can identify laboratory test results even when they are documented using non-standard terminology, abbreviated test names, or alternative measurement units. The pattern matching capabilities may utilize fuzzy string matching techniques that can accommodate minor spelling variations, synonym recognition algorithms that identify alternative names for the same laboratory tests, and unit conversion capabilities that normalize measurement values across different laboratory reporting formats. The robust pattern matching may ensure comprehensive identification of relevant laboratory evidence despite variations in documentation practices and data entry procedures across different healthcare systems.

With continued reference to FIG. 3, the searching operation may implement data quality assessment mechanisms that evaluate the completeness and reliability of retrieved laboratory test results based on data source credibility, result validation status, and temporal consistency with documented clinical timelines. The quality assessment may generate confidence scores for retrieved laboratory results based on factors such as the reliability of the data source, the presence of quality control indicators, and the consistency of results with established reference ranges and clinical expectations. The data quality evaluation may enable subsequent processing operations to appropriately weight laboratory evidence based on its reliability and clinical validity.

The step 340 may utilize advanced database query optimization techniques that enable efficient retrieval of laboratory results from large clinical databases containing extensive historical patient data. The query optimization may include indexing strategies that accelerate search operations across laboratory test codes and patient identifiers, parallel query execution that enables concurrent searching across multiple database systems, and result caching mechanisms that store frequently accessed laboratory data to reduce database access latency. The performance optimization may ensure that comprehensive laboratory result searching can be performed within acceptable time constraints for real-time diagnostic validation applications.

As further shown in FIG. 3, the searching operation may implement comprehensive result aggregation processes that collect and organize laboratory test results from multiple sources into structured datasets suitable for subsequent consistency analysis. The aggregation processes may consolidate results from different laboratory systems, resolve conflicts between duplicate test results, and organize temporal sequences of laboratory values to support trend analysis and clinical interpretation. The result aggregation may include data normalization procedures that standardize measurement units, reference ranges, and result formats across different laboratory reporting systems to enable consistent analysis and comparison operations.

The step 340 may include missing data detection algorithms that identify situations where expected laboratory tests are absent from the patient's clinical data based on the laboratory procedures identified through the semantic relationship analysis. The missing data detection may generate comprehensive reports that specify which laboratory tests should be present based on the documented diagnoses and semantic relationships, but are not found in the available clinical data repositories. The missing test identification may provide specific recommendations for additional laboratory testing that may be needed to support or verify documented diagnostic classifications based on the clinical evidence requirements established through the cross-coding mapping and semantic relationship analysis.

Referring again to FIG. 3, the step 350 of determining whether the diagnostic code is consistent with the laboratory test results may implement sophisticated analytical algorithms that systematically analyze the retrieved laboratory test values to evaluate their consistency with the documented diagnostic classification. The determination operation may compare laboratory test results against established reference ranges, diagnostic criteria, and clinical thresholds to classify the results as supporting, contradicting, or providing insufficient evidence for the documented diagnosis. The consistency evaluation may utilize comprehensive clinical knowledge databases that contain evidence-based diagnostic criteria, laboratory interpretation guidelines, and clinical decision support rules that specify the expected laboratory findings for specific medical conditions.

The determination process may implement multi-parameter analysis algorithms that consider multiple laboratory test results simultaneously to provide comprehensive assessment of diagnostic consistency based on the full spectrum of available clinical evidence. The multi-parameter analysis may evaluate patterns of laboratory abnormalities that are characteristic of specific diagnostic conditions, identify combinations of test results that support or contradict particular diagnoses, and assess the overall strength of laboratory evidence based on the number and significance of relevant test findings. The comprehensive analysis approach may provide more reliable diagnostic validation compared to single-test evaluation methods by considering the cumulative evidence provided by multiple laboratory measurements.

As shown in FIG. 3, the step 350 may utilize sophisticated reference range analysis that accommodates institution-specific reference ranges, age-adjusted normal values, gender-specific thresholds, ace and ethnicity, e.g., a genetic variant in the Duffy Antigen Receptor, common in African-Americans but rare in European-Americans, induces a 1-SD drop in the mean neutrophil count and is the basis of benign ethnic neutropenia, and population-specific variations in laboratory test interpretation. The reference range analysis may access comprehensive reference databases that contain normative values for different patient populations, enabling appropriate interpretation of laboratory results based on patient demographics and clinical context. The determination algorithms may implement dynamic reference range selection that automatically applies the most appropriate normative values based on patient characteristics and institutional laboratory standards.

The determination operation may include clinical significance scoring mechanisms that quantify the diagnostic relevance of laboratory test results based on the strength of association between specific test findings and documented diagnoses. The clinical significance scoring may utilize evidence-based medicine databases that contain information about test sensitivity and specificity for particular diagnostic conditions, enabling the determination process to appropriately weight laboratory evidence based on the diagnostic utility of specific tests. The scoring mechanisms may generate quantitative measures of diagnostic consistency that reflect both the magnitude of laboratory abnormalities and their clinical significance for specific medical conditions.

With continued reference to FIG. 3, the step 350 may implement temporal consistency analysis that evaluates the timing relationships between laboratory test results and diagnostic documentation to ensure that the laboratory evidence is clinically relevant for the documented diagnostic timeframe. The temporal analysis may consider the natural history of specific medical conditions, the expected timeline for laboratory abnormalities to develop or resolve, and the clinical context surrounding the diagnostic documentation. The temporal consistency evaluation may identify situations where laboratory results may not be clinically relevant due to timing discrepancies or may suggest alternative diagnostic considerations based on the temporal pattern of laboratory findings.

The determination process may include contradiction detection algorithms that systematically identify situations where laboratory test results directly contradict the documented diagnostic classification based on established clinical criteria and diagnostic guidelines. The contradiction detection may utilize clinical decision support databases that contain explicit rules for laboratory-based diagnostic exclusion, enabling identification of laboratory findings that are incompatible with specific diagnostic codes. The contradiction analysis may generate detailed explanations of the specific laboratory values and clinical criteria that contribute to the identified inconsistencies, providing healthcare providers with clear rationale for diagnostic review or revision.

As further shown in FIG. 3, the step 350 may implement confidence assessment mechanisms that generate reliability scores for diagnostic consistency determinations based on the quality and completeness of available laboratory evidence. The confidence assessment may consider factors such as the number of relevant laboratory tests available, the clinical significance of identified abnormalities, the consistency of results across multiple test parameters, and the temporal relationship between laboratory findings and diagnostic documentation. The confidence scoring may enable healthcare providers to appropriately interpret diagnostic validation results and prioritize review activities based on the reliability of the analytical conclusions.

The determination operation may include differential diagnosis analysis capabilities that evaluate laboratory test results against multiple diagnostic possibilities to identify alternative diagnoses that may be more consistent with the available clinical evidence. The differential diagnosis analysis may utilize comprehensive diagnostic databases that contain laboratory criteria for multiple medical conditions, enabling systematic comparison of documented diagnoses against alternative diagnostic possibilities based on the pattern of laboratory findings. The differential analysis may provide recommendations for additional diagnostic testing or clinical evaluation that may help resolve diagnostic uncertainty or confirm alternative diagnostic considerations.

Referring again to FIG. 3, the step 350 may provide comprehensive result documentation processes that generate detailed analytical reports summarizing the diagnostic consistency evaluation and providing supporting evidence for the determination conclusions. The result documentation may include summaries of laboratory test results analyzed, reference ranges and diagnostic criteria applied, consistency scores generated, and specific findings that support or contradict the documented diagnosis. The comprehensive documentation may enable healthcare providers to understand the analytical logic underlying the diagnostic validation results and make informed decisions about potential diagnostic revisions or additional clinical evaluation requirements.

The determination process may implement iterative analysis capabilities that enable refinement of diagnostic consistency assessments through incorporation of additional laboratory data or updated clinical information. The iterative analysis may support scenarios where initial laboratory evidence is insufficient for definitive diagnostic validation, enabling subsequent analysis cycles that incorporate new test results or additional clinical context. The iterative approach may provide continuous improvement of diagnostic validation accuracy through systematic incorporation of evolving clinical evidence and feedback from healthcare providers regarding the clinical utility and accuracy of diagnostic consistency determinations.

Referring to FIG. 3, the step 360 of responding to the determining operation may provide comprehensive validation result generation processes that create structured outputs presenting the diagnostic consistency evaluation findings in formats suitable for clinical review and decision-making. The responding operation may generate validation results that indicate consistency between the diagnostic code and the laboratory test results through systematic compilation of the analytical findings obtained during the step 350. The validation result generation may organize the diagnostic consistency assessment outcomes into structured reports that summarize the laboratory evidence analyzed, the consistency determinations made, and the clinical significance of the validation findings for healthcare provider review and potential action.

The step 360 may implement structured output generation algorithms that compile the diagnostic consistency evaluation results into comprehensive validation reports containing detailed information about the laboratory test results analyzed, the reference ranges and diagnostic criteria applied during the consistency assessment, and the specific findings that support or contradict the documented diagnosis. The structured outputs may include quantitative consistency scores that reflect the degree of agreement between laboratory evidence and documented diagnoses, confidence indicators that assess the reliability of the validation conclusions, and detailed explanations of the analytical logic underlying the diagnostic consistency determinations. The validation result formatting may organize information according to clinical priority and significance to enable efficient review by healthcare providers.

As shown in FIG. 3, the validation result generation may create comprehensive assessment summaries that present the overall diagnostic validation outcomes in formats that support clinical decision-making processes. The assessment summaries may include executive summaries that highlight the primary validation conclusions, detailed analytical sections that provide supporting evidence and rationale for the consistency determinations, and recommendation sections that suggest potential actions based on the validation findings. The structured presentation may enable healthcare providers to quickly assess the validation outcomes while providing access to detailed analytical information when more comprehensive review is required.

The step 360 may include alert generation processes that create notifications classified according to clinical urgency when inconsistencies are detected between documented diagnoses and available laboratory evidence. The alert generation may implement sophisticated classification algorithms that assess the clinical significance and potential impact of identified inconsistencies to determine appropriate alert priority levels. High-priority alerts may be generated for situations involving significant diagnostic discrepancies that could affect patient safety or treatment decisions, while lower-priority alerts may address documentation quality issues or minor inconsistencies that may not immediately impact patient care but could affect billing accuracy or clinical quality metrics.

With continued reference to FIG. 3, the alert classification system may utilize clinical decision support databases that contain evidence-based criteria for assessing the clinical significance of diagnostic inconsistencies based on the specific medical conditions involved, the magnitude of laboratory abnormalities detected, and the potential impact on patient care outcomes. The classification algorithms may consider factors such as the severity of the documented medical condition, the diagnostic utility of the contradictory laboratory findings, and the availability of alternative diagnostic explanations for the observed laboratory results. The alert prioritization may ensure that healthcare providers receive immediate notification of clinically significant issues while avoiding alert fatigue through appropriate filtering of less urgent validation findings.

The alert generation may create detailed notification messages that include specific recommendations for diagnostic review, additional testing, or documentation clarification based on the nature and significance of the identified inconsistencies. The recommendation generation may utilize clinical guideline databases that contain evidence-based protocols for addressing diagnostic uncertainty, laboratory result interpretation, and clinical decision-making in situations where documented diagnoses may not be fully supported by available clinical evidence. The specific recommendations may include suggestions for additional laboratory testing that could provide clarifying evidence, referrals to clinical specialists who may provide expert consultation on complex diagnostic scenarios, or documentation review processes that could identify potential coding errors or clinical information gaps.

As further shown in FIG. 3, the alert generation may implement recommendation specificity algorithms that tailor the suggested actions to the particular clinical context and diagnostic scenario identified through the validation process. The recommendation algorithms may analyze the specific laboratory abnormalities detected, the documented diagnostic codes involved, and the clinical specialty context to generate targeted suggestions that are most likely to resolve the identified diagnostic inconsistencies. The tailored recommendations may include specific laboratory tests that are most likely to provide diagnostic clarification, clinical assessment procedures that could confirm or refute the documented diagnosis, or documentation review activities that could identify missing clinical information or coding errors.

The step 360 may include inconsistency explanation generation processes that provide detailed descriptions of the specific laboratory values, reference ranges, and clinical criteria that contribute to the identified diagnostic inconsistencies. The explanation generation may create comprehensive analytical narratives that describe the expected laboratory findings for the documented diagnosis, the actual laboratory results observed in the patient's clinical data, and the clinical significance of the discrepancies identified through the consistency analysis. The detailed explanations may enable healthcare providers to understand the analytical rationale underlying the inconsistency detection and make informed decisions about appropriate clinical responses.

Referring again to FIG. 3, the responding operation may implement notification delivery systems that present alerts and validation results through multiple communication channels to ensure that healthcare providers receive timely notification of diagnostic validation findings. The delivery systems may include integration with electronic health record interfaces that present validation results as contextual alerts during clinical documentation review processes, email notification systems that provide detailed validation reports to designated clinical personnel, and mobile notification capabilities that enable immediate alert delivery to healthcare providers regardless of their location within the healthcare facility. The multi-channel delivery approach may ensure that validation findings reach appropriate clinical personnel in formats and timeframes that support effective clinical decision-making.

The step 360 may include alert acknowledgment tracking mechanisms that monitor healthcare provider responses to validation alerts to ensure that identified diagnostic inconsistencies receive appropriate clinical attention. The acknowledgment tracking may record which alerts are reviewed by clinical personnel, which recommendations are accepted or implemented, and which validation findings result in diagnostic revisions or additional clinical evaluation. The tracking information may be utilized to assess the clinical utility of the diagnostic validation process, identify patterns in healthcare provider responses to validation alerts, and continuously improve the accuracy and relevance of inconsistency detection algorithms.

As shown in FIG. 3, the responding operation may implement alert suppression mechanisms that prevent redundant notifications for diagnostic inconsistencies that have already been reviewed or addressed by healthcare providers. The suppression algorithms may track resolved validation issues and avoid generating duplicate alerts for the same diagnostic problems within specified time periods or clinical encounters. The alert suppression may include intelligent filtering capabilities that recognize when diagnostic revisions have been made, additional laboratory testing has been ordered to address identified inconsistencies, or clinical documentation has been updated to resolve validation concerns.

The step 360 may provide customizable notification preferences that enable healthcare organizations and individual providers to configure alert settings according to clinical specialty requirements, institutional protocols, and individual practice patterns. The customization capabilities may include threshold settings that determine the sensitivity of inconsistency detection algorithms, priority classification criteria that specify which types of diagnostic discrepancies warrant immediate notification, and delivery method preferences that specify how validation alerts should be presented to different categories of clinical personnel. The preference customization may enable healthcare organizations to tailor the diagnostic validation system to their specific clinical workflows and quality assurance requirements.

With continued reference to FIG. 3, the validation result generation may include comprehensive reporting capabilities that create detailed analytical reports suitable for quality assurance activities, clinical research applications, and healthcare performance monitoring initiatives. The comprehensive reporting may generate statistical summaries of diagnostic validation outcomes across multiple patients or clinical encounters, trend analysis reports that identify patterns in diagnostic consistency over time, and comparative analysis reports that assess validation performance across different clinical specialties or healthcare providers. The detailed reporting capabilities may support institutional quality improvement initiatives and provide data for clinical research studies focused on diagnostic accuracy and healthcare quality metrics.

The step 360 may implement result archival and retrieval systems that maintain historical records of diagnostic validation outcomes for longitudinal analysis and clinical audit purposes. The archival systems may store validation results in structured databases that enable efficient retrieval and analysis of historical validation data for specific patients, clinical conditions, or healthcare providers. The historical data maintenance may support clinical research activities, quality assurance investigations, and performance improvement initiatives that require access to comprehensive validation outcome data over extended time periods.

As further shown in FIG. 3, the responding operation may include integration capabilities that enable seamless incorporation of diagnostic validation results into existing healthcare information systems and clinical workflow processes. The integration capabilities may include application programming interfaces that enable electronic health record systems to access validation results, clinical decision support system interfaces that incorporate validation findings into broader clinical guidance frameworks, and billing system integration that ensures diagnostic coding accuracy based on validation outcomes. The comprehensive integration approach may ensure that diagnostic validation results are effectively utilized across multiple aspects of healthcare delivery and administration.

The step 360 may provide feedback collection mechanisms that gather input from healthcare providers regarding the accuracy, clinical utility, and workflow impact of diagnostic validation results and alert notifications. The feedback collection may include structured surveys that assess provider satisfaction with validation outcomes, usage analytics that track how validation results are utilized in clinical decision-making processes, and outcome tracking that monitors the clinical impact of validation-driven diagnostic revisions or additional testing. The collected feedback may be utilized to continuously improve the diagnostic validation algorithms, refine alert generation criteria, and enhance the clinical relevance of validation results for healthcare providers.

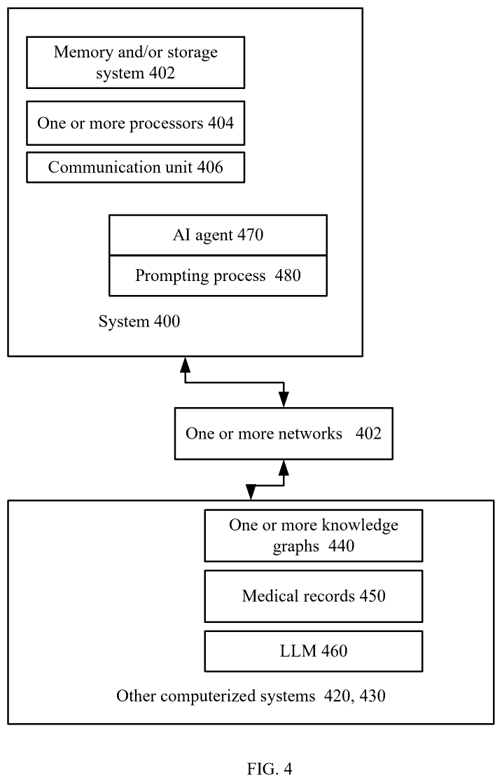

Referring to FIG. 4, a system 400 for providing medical information analysis may provide the hardware and software infrastructure necessary for implementing medical documentation analysis using knowledge graph enhanced language models. The system 400 may coordinate multiple components and external resources to perform comprehensive medical information processing operations that combine natural language processing capabilities with structured medical knowledge retrieval systems. The system 400 may be implemented using various hardware architectures and computing resources that provide the computational capabilities necessary for processing medical documentation and accessing knowledge graph systems while accommodating diverse organizational requirements, security considerations, and performance objectives.

As shown in FIG. 4, the system 400 may include memory and/or storage resources 402 that provide high-capacity data storage for language model parameters, knowledge graph data structures, medical coding databases, patient documentation repositories, and system configuration data. The memory and/or storage resources 402 may include both volatile memory for active processing operations and non-volatile storage for persistent data such as knowledge graphs, medical ontologies, and historical analysis results. The memory and/or storage resources 402 may be configured to provide rapid access to frequently used medical coding systems and knowledge graph structures to support real-time analysis operations during clinical documentation processes.

With continued reference to FIG. 4, the system 400 may include one or more processors 404 that serve as computational components executing various analysis operations, language model processing, and knowledge graph interactions. The one or more processors 404 may be configured to handle natural language processing tasks, execute query operations against knowledge graphs, and perform reasoning operations based on combined information from medical notes and structured medical knowledge. The one or more processors 404 may include central processing units for general-purpose computing tasks, graphics processing units for language model inference operations, tensor processing units for specialized artificial intelligence acceleration, or other processing hardware optimized for different aspects of the medical documentation analysis workflow.

As further shown in FIG. 4, the system 400 may include a communication unit 406 that provides network interfaces enabling high-speed data communication between system components and external resources. The communication unit 406 may support various protocols and data formats to accommodate integration with existing healthcare information systems and electronic medical record platforms. The communication unit 406 may enable the system 400 to receive medical documentation, access remote knowledge graph systems, communicate with external language model services, and deliver analysis results to clinical users and healthcare information systems through bidirectional data exchange capabilities.

Referring again to FIG. 4, the system 400 may communicate with other computerized systems 420 and computerized systems 430 through the communication unit 406 to access distributed resources and external services that support medical documentation analysis operations. The other computerized systems 420 and computerized systems 430 may store, access, or implement various resources including one or more knowledge graphs 440, medical records 450, and LLM 460 that provide specialized capabilities for different aspects of the medical information processing workflow. The distributed architecture may enable the system 400 to leverage external computational resources, specialized knowledge repositories, and cloud-based services while maintaining local control over sensitive medical data and core processing operations.

As shown in FIG. 4, the knowledge graphs 440 may include various medical coding systems and ontologies such as ICD-10 Clinical Modification codes, SNOMED CT, Wikidata, and other standardized medical terminologies stored in different database systems including relational databases, graph databases, and semantic web repositories. The medical records 450 may include electronic health record systems, laboratory information systems, and clinical data repositories that contain medical documentation, laboratory test results, medication histories, and other patient-related information. The LLM 460 may provide natural language processing capabilities for medical entity extraction, text analysis, and clinical reasoning operations through cloud-based services, on-premises language model deployments, or hybrid configurations that combine local and remote processing capabilities.

With continued reference to FIG. 4, the system 400 may implement an AI agent 470 that provides AI agent-based reasoning capabilities enabling dynamic query formulation and complex clinical analysis operations. The AI agent 470 may utilize frameworks such as ReAct or LangChain to coordinate interactions between language models and knowledge graph systems, formulate analytical plans, execute knowledge graph queries as tools, and reason about retrieved information to support complex clinical decision-making processes. The AI agent 470 may enable the system 400 to adapt analytical approaches based on specific clinical contexts and documentation complexity through sophisticated reasoning processes that extend beyond predetermined template-based analysis methods. The AI agent may include the LLM.

As further shown in FIG. 4, the system 400 may implement a prompting process 480 that manages the generation of augmented prompts combining original medical documentation with retrieved knowledge graph information. The prompting process 480 may structure contextual information to support optimal language model comprehension and utilization, format retrieved medical knowledge according to relevance and clinical significance, and coordinate the integration of multiple knowledge sources into comprehensive analytical inputs. The prompting process 480 may ensure that knowledge graph information is effectively integrated with medical documentation to enable enhanced analysis operations that leverage both unstructured clinical text and structured medical knowledge.

Referring to FIG. 4, the system 400 may be implemented using centralized computing systems with dedicated hardware resources for medical documentation analysis within healthcare facilities or data centers. Centralized computing systems may provide dedicated hardware resources including high-performance server hardware configured with multiple processors, substantial memory capacity, and high-speed storage systems to support real-time analysis of medical documentation. The centralized architecture may offer advantages including simplified data management, reduced network latency for accessing local knowledge graph systems, and enhanced data security through physical control of computing resources within healthcare facility boundaries.

The centralized implementation may utilize enterprise-grade server hardware from vendors such as Dell EMC PowerEdge servers, HPE ProLiant systems, or Lenovo ThinkSystem platforms configured with redundant components to ensure continuous availability for clinical operations. The centralized systems may include high-performance processors such as Intel Xeon Scalable processors, AMD EPYC processors, or ARM-based server processors that provide substantial parallel processing capabilities for handling multiple analysis tasks concurrently. The centralized architecture may enable healthcare organizations to maintain direct control over medical data processing while providing predictable performance characteristics and simplified system administration requirements.

As shown in FIG. 4, the system 400 may be implemented using distributed computing architectures that distribute medical documentation analysis tasks across multiple computing nodes for parallel processing capabilities. Distributed computing architectures may enable parallel processing of multiple medical notes simultaneously, reducing overall processing time and supporting high-volume clinical environments through coordinated workload distribution across available computing resources. The distributed architecture may include multiple server nodes coordinated through cluster management software, with workload distribution mechanisms that balance processing tasks across available computing resources to optimize system performance and resource utilization.

The distributed implementation may utilize technologies such as Kubernetes container orchestration, Apache Hadoop distributed processing frameworks, or custom load balancing systems to coordinate analysis operations across computing clusters. The distributed architecture may provide scalable processing capabilities by adding computing resources as needed to accommodate varying processing loads and support healthcare organizations ranging from small clinics processing hundreds of notes daily to large hospital systems analyzing tens of thousands of medical documents each day. The distributed approach may include fault tolerance mechanisms that enable workload redistribution when computing nodes become unavailable, ensuring continuous system operation even when individual hardware components fail.

With continued reference to FIG. 4, the system 400 may be implemented using cloud-based infrastructure utilizing services from providers such as Amazon Web Services, Microsoft Azure, or Google Cloud Platform to access computing, storage, and database resources on demand. Cloud-based infrastructure may provide flexible and scalable computing resources for medical documentation analysis without requiring substantial on-premises hardware investments. The cloud implementations may offer advantages including elastic scalability to accommodate varying processing loads, geographic distribution of computing resources for multi-site healthcare organizations, and access to specialized hardware accelerators for machine learning operations through cloud service provider offerings.

The cloud-based implementation may utilize HIPAA-compliant cloud services that provide appropriate security controls and data protection mechanisms for handling protected health information in accordance with healthcare regulatory requirements. Cloud architectures may enable healthcare organizations to access advanced computational capabilities including high-performance graphics processing units, tensor processing units, and specialized artificial intelligence accelerators without requiring direct hardware procurement and maintenance. The cloud approach may provide cost optimization benefits through pay-per-use pricing models and automatic resource scaling that adjusts computational capacity based on actual processing demand patterns.

As further shown in FIG. 4, the system 400 may be implemented using hybrid computing architectures that combine on-premises hardware resources with cloud-based services to balance data security requirements with scalability needs. Hybrid implementations may maintain sensitive patient data and knowledge graph systems within on-premises infrastructure while utilizing cloud resources for computationally intensive language model processing operations. The hybrid approach may provide flexibility for healthcare organizations to optimize cost, performance, and security considerations based on specific operational requirements and regulatory compliance obligations while leveraging the benefits of both local control and cloud scalability.

The hybrid architecture may enable healthcare organizations to maintain critical medical data within their direct control while accessing cloud-based computational resources for processing operations that do not require local data storage. The hybrid implementation may include secure communication channels between on-premises and cloud components, data encryption mechanisms that protect information during transmission and processing, and access control systems that ensure appropriate authorization for cloud-based processing operations. The hybrid approach may provide optimal balance between security, performance, and cost considerations for healthcare organizations with diverse operational requirements and varying levels of cloud adoption readiness.

Referring again to FIG. 4, the system 400 architecture may support both centralized and distributed implementations of medical note analysis functionality through flexible deployment configurations that accommodate diverse organizational requirements and technical constraints. The system 400 may provide deployment flexibility enabling healthcare organizations to select implementation approaches that align with their existing infrastructure, security policies, and operational requirements while maintaining comprehensive medical documentation analysis capabilities. The architectural flexibility may enable gradual migration between different implementation approaches as organizational requirements evolve or as new technological capabilities become available.

The system 400 may include configuration management capabilities that enable healthcare organizations to customize system behavior, performance characteristics, and integration requirements based on their specific clinical workflows and technical environments. The configuration capabilities may include settings for knowledge graph access methods, language model processing parameters, alert generation criteria, and notification delivery preferences that can be tailored to different clinical specialties and organizational practices. The flexible configuration approach may ensure that the system 400 can be effectively adapted to diverse healthcare environments while maintaining consistent analytical capabilities and clinical utility across different implementation scenarios.

Referring to FIG. 4, the one or more processors 404 may include various types of processing resources optimized for different aspects of the medical documentation analysis workflow to provide comprehensive computational capabilities that support natural language processing, knowledge graph access, and clinical reasoning operations. The processors 404 may be configured to handle diverse computational requirements including general-purpose computing tasks, specialized machine learning inference operations, and high-performance parallel processing activities that enable real-time medical documentation analysis in clinical environments. The processing resources may be selected and configured based on the specific computational demands of different workflow components to optimize overall system performance and analytical accuracy.

The processors 404 may include central processing units for general-purpose computing tasks including data management, workflow coordination, and knowledge graph query operations. Modern multi-core central processing units from vendors such as Intel Xeon Scalable processors, AMD EPYC processors, or ARM-based server processors may provide substantial parallel processing capabilities for handling multiple analysis tasks concurrently. The central processing units may enable efficient processing of multiple medical notes simultaneously, reducing overall analysis time and supporting real-time clinical decision support applications through coordinated execution of workflow management, data processing, and system coordination operations.

As shown in FIG. 4, the processors 404 may include graphics processing units from vendors such as NVIDIA for language model inference operations that benefit from parallel processing architectures. Graphics processing units from NVIDIA, including A100, H100, or L40S models, or AMD Instinct accelerators may provide specialized hardware acceleration for language model inference operations that significantly reduce the time required for natural language processing of medical documentation. The parallel processing architecture of graphics processing units may enable efficient execution of the matrix operations and neural network computations that form the basis of large language model inference, potentially reducing analysis time from minutes to seconds for complex medical notes.

The graphics processing units may provide substantial performance improvements for transformer-based language model architectures through optimized execution of attention mechanisms, matrix multiplication operations, and neural network layer computations that are fundamental to medical entity extraction and text analysis processes. Healthcare organizations processing high volumes of medical documentation may achieve substantial performance improvements by utilizing graphics processing unit acceleration for language model operations, enabling real-time analysis capabilities that support immediate clinical decision support during patient encounters. The specialized parallel processing capabilities may enable the system 400 to maintain multiple concurrent analysis operations while preserving analytical accuracy and response time requirements.

With continued reference to FIG. 4, the processors 404 may include tensor processing units and other specialized artificial intelligence accelerators such as TPUs developed by Google for optimized machine learning inference operations. Tensor processing units may provide power-efficient processing capabilities specifically designed for neural network operations that are commonly utilized in medical documentation analysis workflows. The specialized artificial intelligence accelerators may offer advantages including reduced energy consumption compared to general-purpose graphics processing units, optimized memory architectures for machine learning workloads, and integrated support for common neural network operations that are frequently utilized in natural language processing applications.

The tensor processing units may provide optimized hardware architectures that are specifically designed for the mathematical operations required by transformer-based language models and other neural network architectures used in medical text analysis. The specialized processing units may include dedicated matrix multiplication units, optimized memory hierarchies, and integrated support for common activation functions that enable efficient execution of language model inference operations. Healthcare organizations may utilize tensor processing units to reduce operational costs while maintaining high-performance medical documentation analysis capabilities through energy-efficient processing that reduces power consumption and cooling requirements.

As further shown in FIG. 4, the specialized artificial intelligence accelerators may provide custom processing capabilities that are optimized for specific types of machine learning operations commonly encountered in medical documentation analysis workflows. The custom accelerators may include dedicated hardware for attention mechanism computations, optimized data pathways for sequential processing operations, and specialized memory architectures that minimize data movement during neural network inference. The specialized processing capabilities may enable the system 400 to achieve optimal performance for medical entity extraction, knowledge graph integration, and clinical reasoning operations through hardware architectures that are specifically designed for artificial intelligence workloads.

The processors 404 may provide technical benefits including substantially reduced processing time for medical documentation analysis compared to manual review processes or software-only implementations. Automated analysis using optimized hardware configurations may process complex medical notes in seconds rather than the minutes or hours required for manual coding and verification operations. The rapid processing capabilities may enable real-time clinical decision support during patient encounters, allowing physicians to receive immediate feedback about potential coding errors, diagnostic inconsistencies, or missing clinical information while documentation is being created during clinical workflows.

Referring again to FIG. 4, the processing resources may provide improved analytical accuracy through specialized hardware acceleration that enables more sophisticated language model architectures and comprehensive knowledge graph integration operations. The enhanced computational capabilities may support larger language models with improved medical terminology recognition, more comprehensive knowledge graph access operations, and sophisticated reasoning processes that improve the accuracy of medical entity extraction and clinical relationship identification. The specialized processing hardware may enable the system 400 to utilize state-of-the-art artificial intelligence models that provide superior analytical performance compared to implementations constrained by general-purpose processing limitations.

The processors 404 may provide enhanced scalability benefits that enable healthcare organizations to process increasing volumes of medical documentation without proportional increases in processing time or resource requirements. Distributed computing architectures utilizing multiple graphics processing units or tensor processing units may accommodate growth in documentation volume by adding processing resources as needed, providing flexible capacity management capabilities. The scalable architecture may support healthcare organizations ranging from small clinics processing hundreds of notes daily to large hospital systems analyzing tens of thousands of medical documents each day through coordinated utilization of specialized processing resources.

As shown in FIG. 4, the processing resources may enable parallel processing capabilities that allow simultaneous analysis of multiple medical documents while maintaining consistent analytical quality and response times. The parallel processing architecture may distribute analysis tasks across multiple processing units, enabling concurrent execution of language model inference operations, knowledge graph access procedures, and clinical reasoning processes. The parallel execution capabilities may reduce overall processing latency and improve system throughput, enabling the system 400 to support high-volume clinical environments where multiple healthcare providers require simultaneous access to medical documentation analysis capabilities.

The processors 404 may provide energy efficiency benefits through utilization of specialized artificial intelligence accelerators and optimized hardware configurations that deliver substantially higher computational performance per watt compared to general-purpose processing approaches. Modern tensor processing units and graphics processing units may provide energy-efficient processing capabilities that reduce operational costs and environmental impact while maintaining high-performance medical documentation analysis capabilities. Healthcare organizations may achieve reduced total cost of ownership through energy-efficient hardware selections that minimize power consumption and cooling requirements while providing superior analytical performance compared to traditional computing architectures.

Referring to FIG. 4, a memory and storage system 402 may provide high-capacity data storage for language model parameters, knowledge graph data structures, medical coding databases, patient documentation repositories, and system configuration data that support comprehensive medical documentation analysis operations. The memory and storage system 402 may include both volatile memory for active processing operations and non-volatile storage for persistent data such as knowledge graphs, medical ontologies, and historical analysis results. The memory and storage system 402 may be configured to provide rapid access to frequently used medical coding systems and knowledge graph structures to support real-time analysis operations during clinical documentation processes while maintaining data integrity and system reliability through advanced storage technologies and memory architectures.

As shown in FIG. 4, the memory and storage system 402 may include volatile memory components that provide high-speed data access for active processing operations including language model inference, knowledge graph queries, and intermediate analysis results. The volatile memory may store language model parameters during inference operations, maintain knowledge graph data structures in memory for rapid access, and provide temporary storage for processing results during multi-step analysis workflows. The volatile memory components may utilize high-capacity configurations ranging from hundreds of gigabytes to multiple terabytes to support large language model inference and knowledge graph operations that require substantial memory resources for optimal performance.

The memory and storage system 402 may include error-correcting code memory using DDR4 or DDR5 memory modules from vendors such as Samsung, Micron, or SK Hynix that provide data integrity protection during processing operations. Error-correcting code memory may detect and correct memory errors that could affect medical documentation analysis accuracy, preventing data corruption that could impact clinical decision support outcomes. The error-correcting code capabilities may be particularly important for healthcare applications where data integrity directly affects patient care quality and clinical decision-making processes. The memory modules may support high-capacity configurations using registered memory modules that enable substantial memory capacity while maintaining system stability and reliability.

With continued reference to FIG. 4, the memory and storage system 402 may utilize multi-channel memory configurations that provide increased memory bandwidth to support the high data throughput requirements of language model inference and knowledge graph access operations. The multi-channel memory architecture may enable parallel data access across multiple memory channels, reducing memory access latency and improving overall system performance during concurrent processing operations. The memory bandwidth optimization may be particularly beneficial for transformer-based language model architectures that require rapid access to large parameter datasets (also referred to as weights or checkpoints) during inference operations and knowledge graph systems that benefit from high-speed data retrieval capabilities.

The memory and storage system 402 may include non-volatile storage components that provide persistent data storage for knowledge graphs, medical coding databases, patient documentation repositories, and system configuration data. The non-volatile storage may maintain comprehensive medical knowledge repositories including ICD-10 Clinical Modification codes, SNOMED CT terminologies, pharmaceutical databases, and laboratory test classifications that support enhanced medical documentation analysis. The persistent storage capabilities may ensure that medical knowledge remains available across system restarts and provide reliable data retention for historical analysis results and system configuration parameters.

As further shown in FIG. 4, the memory and storage system 402 may include solid-state drives using NVMe interfaces from vendors such as Samsung, Intel, Micron, or Western Digital that provide high-performance storage capabilities for knowledge graph data and medical coding databases. The solid-state drives may offer substantially higher data access speeds compared to traditional hard disk drives, enabling rapid retrieval of medical coding information and knowledge graph structures during real-time analysis operations. The NVMe interface technology may provide parallel data access capabilities that support concurrent knowledge graph queries from multiple analysis operations, reducing storage access latency and improving overall system responsiveness.

The solid-state drives may utilize enterprise-grade storage technologies that provide enhanced reliability, endurance, and performance characteristics suitable for healthcare applications that require continuous operation and high data integrity standards. The enterprise solid-state drives may include advanced wear leveling algorithms that distribute write operations across storage cells to maximize device lifespan, power loss protection mechanisms that prevent data corruption during unexpected power interruptions, and enhanced error correction capabilities that maintain data integrity under demanding operational conditions. The high-performance storage capabilities may enable the system 400 to maintain large knowledge graph datasets while providing rapid access during real-time medical documentation analysis operations.

Referring again to FIG. 4, the memory and storage system 402 may implement redundant storage configurations using RAID arrays or distributed storage systems that provide data protection and ensure continuous availability of knowledge graph systems and medical databases. The redundant storage architectures may protect against data loss due to individual storage device failures while maintaining system availability during storage maintenance operations. The redundancy mechanisms may include mirroring configurations that maintain duplicate copies of critical medical knowledge databases, striping configurations that distribute data across multiple storage devices for improved performance, and parity-based protection schemes that enable data recovery from storage device failures.

The memory and storage system 402 may include storage area networks or network-attached storage systems that enable centralized management of knowledge graph data while providing high-speed access from multiple computing nodes in distributed architectures. The networked storage approach may enable multiple processors 404 to access shared medical knowledge repositories simultaneously while maintaining data consistency and access coordination. The centralized storage management may simplify knowledge graph updates, backup operations, and system maintenance activities while providing scalable storage capacity that can accommodate growing medical knowledge databases and expanding clinical documentation repositories.

As shown in FIG. 4, the memory and storage system 402 may store instructions that, when executed by the processors 404, cause the system 400 to perform comprehensive medical documentation analysis operations including receiving medical documentation, processing documents with language models, accessing knowledge graphs, generating augmented contexts, and producing enhanced analysis results. The stored instructions may implement the software components necessary for coordinating interactions between language model processing, knowledge graph access, and clinical decision support operations. The instruction storage may include executable code for implementing AI agent-based reasoning capabilities, template-based query systems, and real-time notification mechanisms that support diverse clinical workflows and analytical requirements.

The stored instructions may configure the processors 404 to execute sophisticated analytical workflows that combine natural language processing capabilities with structured medical knowledge retrieval to generate enhanced clinical insights and decision support recommendations. The instruction implementation may include software modules for medical entity extraction, cross-coding system mapping, diagnostic validation, and consistency checking operations that leverage the comprehensive medical knowledge stored within the memory and storage system 402. The executable instructions may enable the system 400 to adapt analytical approaches based on clinical context, documentation complexity, and organizational requirements while maintaining consistent analytical accuracy and clinical utility.

With continued reference to FIG. 4, the memory and storage system 402 may implement caching mechanisms that store frequently accessed medical knowledge in high-speed memory to reduce storage access latency during repeated analysis operations. The caching systems may maintain hierarchical code structures, semantic relationship data, and cross-referencing information in volatile memory to enable rapid retrieval during subsequent analysis operations. The cache management may include automatic cache invalidation processes that ensure cached medical knowledge remains current with updates to the underlying knowledge graph systems and medical coding databases stored in non-volatile storage components.

The memory and storage system 402 may provide performance optimization capabilities through intelligent data placement strategies that position frequently accessed medical knowledge in high-speed storage tiers while maintaining less frequently used data in cost-effective storage systems. The tiered storage approach may automatically migrate data between different storage performance levels based on access patterns and analytical requirements, optimizing both system performance and storage cost efficiency. The performance optimization may include predictive caching algorithms that anticipate knowledge graph access patterns based on clinical workflows and preload relevant medical knowledge into high-speed memory before analysis operations begin.

As further shown in FIG. 4, the memory and storage system 402 may implement comprehensive backup and disaster recovery capabilities that protect critical medical knowledge databases and system configuration data against data loss or system failures. The backup systems may maintain multiple copies of knowledge graph data, medical coding databases, and historical analysis results in geographically distributed storage locations to ensure data availability during emergency situations or system maintenance activities. The disaster recovery mechanisms may include automated failover capabilities that redirect processing operations to backup storage systems when primary storage components become unavailable, ensuring continuous system operation and clinical decision support availability.

The memory and storage system 402 may provide data encryption capabilities that protect sensitive medical knowledge and patient information during storage and processing operations. The encryption mechanisms may utilize hardware-based encryption engines that provide transparent data protection without affecting system performance, ensuring that medical knowledge databases and analysis results remain secure while maintaining rapid access capabilities. The encryption implementation may include key management systems that control access to encrypted data based on user authentication and authorization policies, providing comprehensive data protection that meets healthcare security requirements and regulatory compliance standards.

Referring to FIG. 4, the communication unit 406 may provide comprehensive network interfaces that enable high-speed data communication between system components and external resources through advanced networking technologies and protocols designed to support healthcare information exchange requirements. The communication unit 406 may implement sophisticated networking capabilities that accommodate integration with existing healthcare information systems and electronic medical record platforms while maintaining data security, transmission reliability, and performance optimization necessary for real-time medical documentation analysis operations. The communication unit 406 may support various protocols and data formats to ensure seamless interoperability with diverse healthcare technology environments and clinical workflow systems.

As shown in FIG. 4, the communication unit 406 may include high-bandwidth network connections using 10 Gigabit Ethernet networking technologies that provide substantial data transmission capabilities for supporting concurrent access to knowledge graphs 440, medical records 450, and LLM 460 across distributed computing environments. The 10 Gigabit Ethernet implementation may enable rapid transfer of large medical datasets, comprehensive knowledge graph queries, and real-time language model processing requests without introducing significant network latency that could affect clinical workflow performance. The high-bandwidth networking may support multiple concurrent connections to external systems while maintaining consistent data transmission speeds and reliable communication channels for healthcare information exchange operations.

The communication unit 406 may implement 25 Gigabit Ethernet or faster networking technologies that provide enhanced data transmission capabilities for high-volume medical documentation processing environments and large-scale healthcare organizations. The 25 Gigabit Ethernet implementation may enable substantially increased data throughput compared to standard networking technologies, supporting rapid transfer of comprehensive medical knowledge databases, large language model parameters, and extensive patient documentation repositories. The faster networking technologies may accommodate growing healthcare data volumes and increasing computational demands associated with advanced artificial intelligence processing while maintaining optimal system performance and response times.

With continued reference to FIG. 4, the communication unit 406 may include redundant network paths that provide fault tolerance and continuous connectivity capabilities even when individual network components experience failures or maintenance requirements. The redundant networking architecture may implement multiple independent network connections that enable automatic failover capabilities when primary network paths become unavailable, ensuring continuous access to the knowledge graphs 440, medical records 450, and LLM 460 during critical clinical operations. The redundant network paths may utilize diverse routing mechanisms and alternative network infrastructure to minimize the risk of simultaneous network failures that could disrupt medical documentation analysis operations.

The communication unit 406 may implement load balancing capabilities that distribute network traffic across multiple network connections and communication channels to optimize data transmission performance and prevent network congestion during high-volume processing operations. The load balancing mechanisms may automatically distribute communication requests across available network paths based on current network utilization, connection reliability, and transmission performance characteristics. The load balancing implementation may enable the system 400 to maintain optimal network performance even during peak usage periods when multiple healthcare providers require simultaneous access to medical documentation analysis capabilities.

As further shown in FIG. 4, the communication unit 406 may support various communication protocols including TCP/IP for reliable data transmission, HTTP/HTTPS for web-based service integration, and specialized healthcare communication protocols such as HL7 FHIR for standardized medical information exchange. The protocol support may enable the system 400 to communicate effectively with diverse healthcare information systems including electronic health record platforms, laboratory information management systems, and clinical decision support applications. The comprehensive protocol implementation may ensure that the communication unit 406 can accommodate existing healthcare technology infrastructure while supporting emerging standards and communication requirements.

The communication unit 406 may implement secure communication capabilities including encryption protocols, authentication mechanisms, and access control systems that protect sensitive medical information during transmission between system components and external resources. The secure communication implementation may utilize industry-standard encryption algorithms to protect data in transit and/or at rest, certificate-based authentication to verify the identity of communicating systems, and role-based access controls that ensure appropriate authorization for different types of medical information access. The security mechanisms may comply with healthcare regulatory requirements including HIPAA and/or ISO 27001 and/or ISO 27799 privacy and security standards while maintaining efficient data transmission capabilities.

Referring again to FIG. 4, the communication unit 406 may provide application programming interface capabilities that enable standardized integration with external healthcare systems and third-party medical applications. The application programming interface implementation may offer RESTful web services, SOAP-based communication methods, and custom integration protocols that accommodate diverse system architectures and integration requirements. The standardized interface capabilities may enable healthcare organizations to integrate the system 400 with existing clinical workflow systems, billing platforms, and quality assurance applications while maintaining consistent data exchange formats and communication protocols.

The communication unit 406 may implement quality of service mechanisms that prioritize critical medical communication traffic and ensure reliable data transmission for time-sensitive clinical operations. The quality of service implementation may allocate network bandwidth based on communication priority levels, ensuring that urgent medical documentation analysis requests receive preferential network access compared to routine administrative communications. The traffic prioritization may include mechanisms for identifying critical clinical communications, reserving network capacity for high-priority operations, and managing network congestion to maintain optimal performance for essential healthcare information exchange activities.

As shown in FIG. 4, the communication unit 406 may support real-time streaming capabilities that enable continuous data exchange with external systems for immediate processing of medical documentation as documents are created or modified within healthcare information systems. The streaming implementation may utilize persistent network connections, event-driven communication protocols, and low-latency data transmission mechanisms that minimize delays between document creation and analysis completion. The real-time capabilities may enable the system 400 to provide immediate clinical decision support during patient encounters by processing medical documentation as healthcare providers create clinical notes and documentation entries.

The communication unit 406 may include network monitoring and diagnostic capabilities that continuously assess communication performance, identify potential network issues, and provide automated troubleshooting mechanisms for maintaining optimal connectivity with external resources. The monitoring implementation may track network latency, bandwidth utilization, connection reliability, and error rates to ensure consistent communication performance. The diagnostic capabilities may include automated alert generation when network performance degrades below acceptable thresholds, detailed logging of communication activities for troubleshooting purposes, and performance analytics that enable proactive network optimization and capacity planning activities.

With continued reference to FIG. 4, the communication unit 406 may provide scalable networking architecture that accommodates growing communication requirements as healthcare organizations expand their use of medical documentation analysis capabilities. The scalable implementation may support dynamic addition of network connections, automatic bandwidth allocation based on demand patterns, and flexible routing configurations that adapt to changing communication requirements. The scalability features may enable the system 400 to accommodate increasing numbers of concurrent users, expanding integration with additional healthcare systems, and growing volumes of medical documentation processing without requiring fundamental changes to the underlying network architecture.

The communication unit 406 may implement comprehensive error handling and recovery mechanisms that maintain reliable communication even when network conditions are suboptimal or when external systems experience temporary availability issues. The error handling implementation may include automatic retry mechanisms for failed communication attempts, intelligent routing that bypasses problematic network segments, and graceful degradation capabilities that maintain partial functionality when complete communication capabilities are not available. The recovery mechanisms may ensure that critical medical documentation analysis operations can continue even during network maintenance activities or temporary system outages affecting external resources.

Referring to FIG. 4, the computerized systems 420 and computerized systems 430 may represent external entities that store, access, or implement various resources that support comprehensive medical documentation analysis operations performed by the system 400. The computerized systems 420 and computerized systems 430 may provide distributed computing capabilities, specialized knowledge repositories, and external services that extend the analytical capabilities of the system 400 beyond local processing resources. The external systems may enable the system 400 to access comprehensive medical knowledge databases, utilize cloud-based language model services, and integrate with existing healthcare information infrastructure while maintaining local control over sensitive processing operations and clinical workflow coordination.

As shown in FIG. 4, the computerized systems 420 and computerized systems 430 may be implemented using various computing architectures including dedicated database servers, cloud-based service platforms, distributed computing clusters, or hybrid infrastructure configurations that combine on-premises and cloud resources. The external systems may provide specialized computational capabilities that complement the local processing resources of the system 400, enabling access to large-scale knowledge repositories, high-performance language model services, and comprehensive medical record databases that may exceed the storage or processing capacity of individual healthcare organizations. The distributed architecture may enable healthcare organizations to leverage external expertise and computational resources while maintaining appropriate data security and regulatory compliance for sensitive medical information.

With continued reference to FIG. 4, the computerized systems 420 and computerized systems 430 may communicate with the system 400 through the communication unit 406 using standardized protocols and secure communication channels that ensure reliable data exchange while protecting sensitive medical information during transmission. The communication between the system 400 and external systems may utilize encrypted communication protocols, authenticated connection mechanisms, and access control systems that comply with healthcare regulatory requirements including HIPAA privacy and security standards. The secure communication implementation may enable the system 400 to access external resources while maintaining data protection standards appropriate for healthcare applications and clinical decision support operations.

As further shown in FIG. 4, the knowledge graphs 440 may be stored, accessed, or implemented by the computerized systems 420 and computerized systems 430 to provide comprehensive medical coding systems and ontologies that support enhanced medical documentation analysis operations. The knowledge graphs 440 may include various medical coding systems such as ICD-10 Clinical Modification codes that provide diagnostic classifications used for medical billing and clinical documentation purposes, SNOMED CT codes that offer comprehensive clinical terminology systems representing medical concepts and their relationships in detailed semantic structures, and other standardized medical terminologies including CPT procedure codes, LOINC laboratory test codes, and RxNorm medication identifiers. The knowledge graphs 440 may be organized in hierarchical structures, semantic relationship networks, or cross-referenced coding systems that enable systematic querying and retrieval of contextual information to support enhanced language model analysis operations.

The knowledge graphs 440 may be maintained by specialized medical terminology organizations, healthcare technology vendors, or government agencies that provide authoritative sources of medical coding standards and clinical knowledge. The external knowledge graph systems may include comprehensive databases maintained by organizations such as the National Library of Medicine, the International Health Terminology Standards Development Organisation, or commercial medical knowledge providers that offer specialized clinical decision support databases. The external knowledge repositories may provide access to continuously updated medical knowledge that reflects current clinical practices, emerging medical terminology, and evolving diagnostic criteria that may not be available through local knowledge storage systems.

Referring again to FIG. 4, the knowledge graphs 440 may be accessed by the system 400 through the communication unit 406 using various query languages and data access protocols including SQL for relational database implementations, Cypher for property graph systems, SPARQL for RDF-based knowledge sources, or custom application programming interfaces for specialized medical knowledge representations. The knowledge graph access may utilize the AI agent 470 to formulate dynamic queries based on extracted medical entities and clinical context, or may employ predetermined query templates managed by the prompting process 480 for efficient retrieval of commonly accessed medical knowledge. The external knowledge graph access may enable the system 400 to leverage comprehensive medical knowledge repositories without requiring local storage of extensive medical coding databases or semantic relationship networks.

The knowledge graphs 440 may provide biomedical knowledge that enables the system 400 to obtain contextual information related to extracted medical entities through systematic retrieval of hierarchical code structures, semantic relationships, and clinical criteria associated with identified medical concepts. The biomedical knowledge graphs may contain structured representations of medical concepts, relationships between different medical entities, and clinical guidelines that support accurate medical entity classification and diagnostic coding verification. The contextual information retrieval may include cross-referencing operations between different medical coding systems, identification of expected laboratory tests for specific diagnoses, and semantic relationship exploration that supports clinical reasoning and consistency validation operations.

As shown in FIG. 4, the medical records 450 may be stored, accessed, or implemented by the computerized systems 420 and computerized systems 430 to provide comprehensive patient medical files and clinical data repositories that support medical documentation analysis operations performed by the system 400. The medical records 450 may include electronic health record systems that maintain structured patient information including demographic data, medical history, diagnostic codes, medication lists, and clinical assessments. The medical records 450 may also include laboratory information management systems that store test results, reference ranges, and clinical interpretations, as well as clinical data warehouses that aggregate patient information from multiple healthcare sources to provide comprehensive longitudinal medical records.

The medical records 450 may be maintained by healthcare organizations, hospital systems, clinic networks, or health information exchanges that provide centralized access to patient medical information across multiple care providers and clinical settings. The external medical record systems may include comprehensive patient databases that contain historical medical documentation, current clinical assessments, and ongoing treatment information that supports continuity of care and clinical decision-making processes. The medical record repositories may provide access to extensive patient populations and diverse clinical scenarios that enable comprehensive medical documentation analysis across different medical specialties and healthcare environments.

With continued reference to FIG. 4, the medical records 450 may be accessed by the system 400 through the communication unit 406 to receive medical documentation from patient medical files for processing and analysis operations. The medical documentation retrieval may utilize standardized healthcare communication protocols such as HL7 FHIR, secure database connections, or custom integration interfaces that enable systematic access to patient medical information while maintaining appropriate privacy protections and access controls. The medical documentation access may include real-time retrieval of newly created clinical notes, batch processing of historical medical records, or streaming access to medical documentation as documents are created or modified within healthcare information systems.

The system 400 may receive medical documentation from the medical records 450 in various formats including structured electronic health record data, unstructured clinical text, standardized medical messaging formats, and multimedia clinical content such as medical images or diagnostic reports. The medical documentation retrieval may include document validation processes that verify data integrity and completeness, format standardization operations that convert diverse document formats into consistent data structures, and content preparation procedures that organize medical information for subsequent language model processing operations. The comprehensive medical documentation access may enable the system 400 to analyze diverse types of clinical content while maintaining compatibility with existing healthcare information systems and clinical workflow processes.

As further shown in FIG. 4, the LLM 460 may be provided by the computerized systems 420 and computerized systems 430 to offer natural language processing capabilities for medical entity extraction, text analysis, and clinical reasoning operations that support enhanced medical documentation analysis. The LLM 460 may be implemented as cloud-based language model services provided by artificial intelligence technology companies, specialized medical natural language processing platforms offered by healthcare technology vendors, or custom language model deployments maintained by research institutions or healthcare organizations. The external language model services may provide access to large-scale transformer-based architectures that have been trained on extensive medical literature, clinical documentation datasets, and healthcare-specific terminology to develop domain-aware text analysis capabilities.

The LLM 460 may provide sophisticated natural language processing capabilities that exceed the computational resources available to individual healthcare organizations through access to large-scale language models with billions or trillions of parameters. The external language model services may offer specialized medical language models that have been fine-tuned for specific clinical domains, medical specialties, or healthcare applications to provide enhanced accuracy for medical entity extraction and clinical text analysis operations. The cloud-based language model access may enable healthcare organizations to utilize state-of-the-art artificial intelligence capabilities without requiring substantial local computational infrastructure or specialized machine learning expertise.

Referring again to FIG. 4, the LLM 460 may be accessed by the system 400 through the communication unit 406 to process medical documentation and extract medical entities using large language model capabilities provided by external computational resources. The language model access may utilize application programming interfaces that enable the system 400 to submit medical documentation for analysis and receive structured outputs containing identified medical entities, classification labels, and confidence scores. The external language model processing may be coordinated by the AI agent 470 to formulate appropriate analysis requests based on clinical context and analytical objectives, or may be managed by the prompting process 480 to generate augmented prompts that combine medical documentation with contextual information retrieved from the knowledge graphs 440.

The system 400 may process the medical documentation to extract medical entities using the large language model capabilities provided by the LLM 460 through sophisticated natural language processing operations that identify and classify medical concepts within clinical text. The medical entity extraction may utilize transformer-based architectures or other neural network approaches that have been specifically trained on medical terminology, clinical abbreviations, and healthcare-specific language patterns to provide domain-aware processing capabilities. The external language model processing may identify various types of medical entities including diagnoses, medications, laboratory results, and medical procedures through named entity recognition techniques that classify different portions of medical text according to predefined medical entity categories.

As shown in FIG. 4, the integration between the system 400 and the external computerized systems 420 and computerized systems 430 may enable comprehensive medical documentation analysis workflows that leverage distributed computational resources, specialized knowledge repositories, and external expertise while maintaining local control over clinical workflow coordination and sensitive data processing operations. The distributed architecture may provide scalability benefits that enable healthcare organizations to access advanced analytical capabilities without requiring substantial local infrastructure investments, while maintaining appropriate data security and regulatory compliance through secure communication protocols and access control mechanisms implemented by the communication unit 406.

The external system integration may enable the system 400 to adapt to evolving healthcare technology environments and emerging artificial intelligence capabilities through flexible communication interfaces that can accommodate new external services and knowledge resources as they become available. The modular architecture may support gradual migration between different external service providers, integration with emerging medical knowledge standards, and adoption of advanced language model technologies without requiring fundamental changes to local system components or clinical workflow processes. The external system integration approach may provide healthcare organizations with access to cutting-edge medical analysis capabilities while maintaining operational flexibility and technology evolution pathways that support long-term strategic objectives and clinical improvement initiatives.

Referring to FIG. 4, the AI agent 470 may provide sophisticated AI agent-based reasoning capabilities that enable dynamic query formulation and complex clinical analysis operations through advanced artificial intelligence frameworks designed to coordinate interactions between language models and knowledge graph systems. The AI agent may include or be in communication with a router. The router accepts request and routes them to the appropriate component. E.g. a router may decide whether a given paragraph requires accessing a KG for additional context. To determine whether an entity tepe classification request requires a state-of-the-art LLM, or can be answered by a simpler ML model. The AI agent 470 may implement sophisticated reasoning processes that extend beyond predetermined template-based analysis methods by enabling adaptive analytical approaches based on specific clinical contexts and documentation complexity. The AI agent 470 may utilize multiple specialized AI agent architectures that work collaboratively to perform comprehensive medical documentation analysis operations including medical entity extraction, diagnostic validation, medication verification, and clinical consistency assessment through coordinated reasoning and action execution cycles.

As shown in FIG. 4, the AI agent 470 may utilize frameworks such as ReAct that enable language model AI agents to alternate between reasoning steps and action execution phases during complex medical analysis tasks. The ReAct framework may provide structured approaches for combining reasoning and action capabilities, enabling the AI agent 470 to formulate analytical plans based on medical documentation content, execute knowledge graph queries as tools to retrieve relevant medical knowledge, and reason about retrieved information to support sophisticated clinical assessments. During reasoning phases, the AI agent 470 may analyze medical documentation to identify relevant clinical questions, evaluate potential knowledge graph sources, and formulate plans for knowledge graph exploration and verification processes that address specific analytical objectives based on the clinical context and documentation characteristics.

The ReAct framework implementation within the AI agent 470 may support action phases that involve execution of specific knowledge graph queries, retrieval of medical knowledge from selected repositories, and processing of query results to inform subsequent reasoning steps. The iterative nature of the ReAct framework may enable multi-step analysis processes where the AI agent 470 refines understanding through successive cycles of reasoning and knowledge graph interaction. The framework may enable the AI agent 470 to modify analytical approaches based on intermediate results, pursue alternative query strategies when initial approaches yield insufficient information, and integrate information from multiple knowledge graph sources to develop comprehensive clinical assessments that support enhanced medical documentation analysis operations.

With continued reference to FIG. 4, the AI agent 470 may utilize libraries for building and deploying AI agents, such as LangChain that provide comprehensive frameworks for building AI agent-based medical analysis systems that integrate language models with knowledge graph tools and external medical resources. The LangChain libraries may offer pre-built components for AI agent construction, tool integration, and workflow management that simplify the development of complex medical analysis applications within the AI agent 470. The LangChain framework may provide unified interfaces that enable the AI agent 470 to interact with diverse knowledge graph types including relational databases, property graphs, and semantic web resources without requiring specialized knowledge of each query language or data format.

The LangChain implementation may support tool interfaces that abstract the underlying query languages and data access methods used by different knowledge graph systems accessed through the computerized systems 420 and computerized systems 430. The tool interfaces may enable the AI agent 470 to formulate queries using natural language descriptions of information requirements, which are then automatically translated into appropriate query languages such as SQL for relational databases, Cypher for property graphs, or SPARQL for semantic web resources. The abstraction provided by LangChain tool interfaces may enable the AI agent 470 to focus on clinical reasoning and analytical logic rather than technical details of knowledge graph access and query formulation, thereby improving the efficiency and accuracy of medical documentation analysis operations.

As further shown in FIG. 4, the AI agent 470 may implement multiple types of specialized AI agents that are configured to perform distinct analytical tasks within the medical documentation analysis workflow. The specialized AI agent architecture may enable parallel processing of different analytical tasks and provide specialized expertise for complex medical documentation scenarios that require coordinated analysis across multiple clinical domains. The multiple types of specialized AI agents may include diagnostic coding AI agents that focus on accurate assignment and verification of diagnostic codes, medication verification AI agents that analyze medication-related information for accuracy and appropriateness, laboratory analysis AI agents that verify laboratory test orders and interpret results, consistency validation AI agents that perform cross-checking operations across different documentation elements, and differential diagnosis AI agents that support clinical reasoning processes through exploration of alternative diagnostic possibilities.

Referring again to FIG. 4, the diagnostic coding AI agents within the AI agent 470 may focus on accurate assignment and verification of diagnostic codes from medical documentation through specialized training on diagnostic terminology, coding guidelines, and clinical criteria. The diagnostic coding AI agents may be configured to analyze clinical descriptions, identify relevant diagnostic entities, and select appropriate ICD-10-CM or SNOMED CT codes based on documented clinical presentations. The diagnostic coding AI agents may interact with the knowledge graphs 440 accessed through the computerized systems 420 and computerized systems 430 to retrieve hierarchical code structures and verify the specificity and accuracy of code assignments through systematic comparison of clinical documentation with coding criteria. The diagnostic coding AI agents may be configured to recognize anatomical locations, clinical modifiers, and severity indicators that affect diagnostic code selection, enabling precise diagnostic classification that accurately reflects documented clinical conditions.

The medication verification AI agents within the AI agent 470 may specialize in analyzing medication-related information within medical documentation to verify prescription accuracy, identify potential drug interactions, and ensure therapeutic appropriateness. The medication verification AI agents may access pharmaceutical knowledge graphs through the computerized systems 420 and computerized systems 430 to retrieve medication information including therapeutic classifications, dosage guidelines, contraindications, and interaction data. The medication verification AI agents may compare documented prescriptions against patient diagnoses to verify therapeutic appropriateness and identify potential prescribing errors or contraindications that warrant clinical review and possible medication adjustments. The medication verification AI agents may utilize comprehensive pharmaceutical databases to perform systematic verification of medication dosages, administration routes, and treatment durations based on established clinical guidelines and therapeutic protocols.

As shown in FIG. 4, the laboratory analysis AI agents within the AI agent 470 may focus on verification of laboratory test orders and interpretation of laboratory results in relation to documented diagnoses through systematic analysis of clinical evidence requirements. The laboratory analysis AI agents may interact with laboratory knowledge graphs accessed through the computerized systems 420 and computerized systems 430 to identify expected tests based on diagnostic codes, verify the presence of appropriate laboratory data, and detect missing or contradictory test results. The laboratory analysis AI agents may be trained to recognize relationships between specific diagnoses and associated laboratory procedures through semantic relationship exploration in medical knowledge graphs, enabling systematic verification of diagnostic consistency and identification of potential gaps in clinical assessment that may affect diagnostic accuracy or treatment planning.

The consistency validation AI agents within the AI agent 470 may perform comprehensive cross-checking operations across different elements of medical documentation to identify potential inconsistencies or contradictions that may indicate documentation errors or clinical issues requiring physician review. The consistency validation AI agents may analyze relationships between diagnoses, medications, laboratory results, and procedures to detect logical inconsistencies or missing clinical information based on established medical knowledge and clinical guidelines. The consistency validation AI agents may utilize multiple knowledge graph sources simultaneously through the computerized systems 420 and computerized systems 430 to perform comprehensive validation operations that span different medical knowledge domains, enabling detection of subtle inconsistencies that may not be apparent through single-domain analysis approaches.

With continued reference to FIG. 4, the differential diagnosis AI agents within the AI agent 470 may support clinical reasoning processes by exploring alternative diagnostic possibilities based on documented symptoms and clinical findings through systematic analysis of medical knowledge repositories. The differential diagnosis AI agents may query disease knowledge graphs accessed through the computerized systems 420 and computerized systems 430 to identify conditions with similar clinical presentations and retrieve comparative diagnostic criteria. The differential diagnosis AI agents may be configured to perform multi-hop reasoning operations that explore complex relationships between symptoms, diseases, and diagnostic tests across multiple knowledge graph systems, enabling comprehensive exploration of diagnostic alternatives and supporting evidence that assists healthcare providers in clinical decision-making processes.

The differential diagnosis AI agents may perform differential diagnosis analysis by querying the knowledge graphs 440 for diseases with similar symptoms or characteristics to extracted medical entities through sophisticated semantic relationship exploration and clinical pattern matching algorithms. The differential diagnosis analysis may utilize comprehensive disease databases that contain symptom profiles, clinical presentations, and diagnostic criteria for multiple medical conditions, enabling systematic comparison of documented clinical findings against alternative diagnostic possibilities. The differential diagnosis AI agents may identify diseases that share similar clinical presentations with documented conditions, retrieve comparative diagnostic criteria that distinguish between related conditions, and provide recommendations for additional clinical assessments or laboratory testing that may help clarify diagnostic uncertainty or confirm specific diagnostic classifications.

As further shown in FIG. 4, the AI agent 470 may implement sophisticated AI agent training processes that utilize supervised learning approaches with annotated medical documentation datasets that include correct coding assignments, verified clinical relationships, and validated analytical outcomes. The supervised learning training may provide the specialized AI agents with labeled examples of medical documentation paired with correct analytical outcomes including accurate diagnostic codes, appropriate medication verifications, and valid consistency assessments. The training datasets may include diverse medical documentation types spanning multiple clinical specialties to ensure broad applicability of trained AI agents across different healthcare domains and documentation styles. The supervised training process may expose AI agents to various documentation styles, clinical abbreviations, and specialty-specific terminology to develop robust analytical capabilities that can handle diverse clinical scenarios and documentation patterns encountered in real-world healthcare environments.

The AI agent 470 may utilize reinforcement learning techniques where AI agents receive feedback based on the accuracy of their analytical outputs and the clinical appropriateness of their recommendations through iterative learning processes that improve performance over time. The reinforcement learning implementation may enable AI agents to receive positive reinforcement when their analytical results match validated clinical assessments and negative feedback when their outputs contain errors or inconsistencies. The reinforcement learning process may enable AI agents to develop optimal query strategies for accessing the knowledge graphs 440 through the computerized systems 420 and computerized systems 430, and refine their reasoning approaches based on accumulated experience with different types of medical documentation and clinical scenarios. The reinforcement learning approach may support continuous improvement of AI agent performance through systematic incorporation of clinical feedback and validation outcomes.

Referring again to FIG. 4, the AI agent 470 may employ multi-task training approaches that enable AI agents to develop capabilities across multiple related analytical tasks simultaneously by training on diverse medical documentation analysis objectives. The multi-task training may improve AI agent generalization capabilities and enable more efficient knowledge transfer between related analytical tasks such as diagnostic coding, medication verification, and laboratory analysis. The multi-task training approach may enable AI agents trained using this methodology to demonstrate improved performance on complex analytical scenarios that require coordination of multiple analytical capabilities and integration of information from diverse medical knowledge domains accessed through the computerized systems 420 and computerized systems 430.

The multi-task training implementation may enable the AI agent 470 to coordinate the activities of multiple specialized AI agents working on different aspects of the same medical documentation analysis task through sophisticated workflow management and information sharing mechanisms. The coordination capabilities may enable AI agents to share intermediate results, coordinate knowledge graph access operations, and integrate their analytical outputs into comprehensive assessment reports that provide holistic evaluation of medical documentation quality and accuracy. The multi-task training approach may enable the AI agent 470 to develop sophisticated reasoning capabilities that can address complex clinical scenarios requiring integration of diagnostic coding accuracy, medication appropriateness assessment, laboratory result validation, and clinical consistency verification through coordinated AI agent collaboration and information synthesis processes.

As shown in FIG. 4, the AI agent 470 may process the medical documentation to extract medical entities using a large language model through sophisticated coordination between AI agent-based reasoning capabilities and natural language processing operations provided by the LLM 460 accessed through the computerized systems 420 and computerized systems 430. The medical entity extraction process may utilize the specialized AI agents within the AI agent 470 to formulate appropriate analysis requests based on clinical context and analytical objectives, coordinate language model processing operations, and interpret extraction results to identify medical entities including diagnoses, medications, laboratory results, and medical procedures. The AI agent 470 may manage the interaction between local processing capabilities provided by the processors 404 and external language model services to optimize extraction accuracy while maintaining efficient processing workflows that support real-time clinical decision support applications.

The AI agent 470 may coordinate the medical entity extraction process through sophisticated prompt engineering techniques that optimize language model performance for specific medical analysis tasks by incorporating clinical context, analytical objectives, and domain-specific instructions. The prompt engineering capabilities may enable the AI agent 470 to generate task-specific instructions, formatting guidelines, and contextual information designed to elicit accurate responses from the LLM 460 for particular types of medical documentation analysis. The AI agent 470 may customize prompting strategies for different medical specialties, documentation types, and analytical objectives to maximize the accuracy and clinical relevance of medical entity extraction operations while maintaining consistency with established medical terminology and coding standards.

With continued reference to FIG. 4, the AI agent 470 may implement iterative processing approaches where initial medical entity extraction results are refined through additional processing cycles that incorporate feedback from knowledge graph validation and clinical consistency assessment operations. The iterative approach may enable the AI agent 470 to improve extraction accuracy through successive refinement cycles that incorporate contextual information retrieved from the knowledge graphs 440, cross-validation against medical coding standards, and consistency checking across multiple extracted entities. The iterative processing capability may support complex clinical scenarios that require multiple analytical perspectives or comprehensive evaluation of alternative entity classifications based on evolving clinical information and knowledge graph resources accessed through the computerized systems 420 and computerized systems 430.

Referring to FIG. 4, the prompting process 480 may provide comprehensive prompt generation and management capabilities that coordinate the integration of original medical documentation with retrieved knowledge graph information to create enhanced analytical inputs for language model processing operations. The prompting process 480 may implement sophisticated prompt engineering techniques that optimize the presentation of medical information and contextual knowledge to support optimal language model comprehension and utilization during medical documentation analysis workflows. The prompting process 480 may manage the complex task of combining unstructured clinical text with structured medical knowledge retrieved from the knowledge graphs 440 through the computerized systems 420 and computerized systems 430 to generate augmented prompts that enable more accurate and comprehensive medical entity extraction and clinical reasoning operations.

As shown in FIG. 4, the prompting process 480 may implement advanced prompt structuring algorithms that organize medical documentation and knowledge graph information into coherent analytical inputs that facilitate systematic language model processing. The prompt structuring capabilities may analyze the original medical documentation to identify key clinical elements, assess the relevance and clinical significance of retrieved knowledge graph information, and organize the combined content into logical sequences that support optimal language model comprehension. The prompting process 480 may utilize template-based structuring approaches for routine analysis scenarios where prompt organization patterns can be predetermined, as well as dynamic structuring algorithms that adapt prompt organization based on the specific characteristics of medical documentation and the complexity of retrieved contextual information.

The prompting process 480 may coordinate with the AI agent 470 to determine appropriate prompt generation strategies based on the analytical objectives, clinical context, and complexity of medical documentation being processed. The coordination between the prompting process 480 and the AI agent 470 may enable adaptive prompt generation that considers the specific requirements of different specialized AI agents including diagnostic coding AI agents, medication verification AI agents, laboratory analysis AI agents, and consistency validation AI agents. The prompting process 480 may generate specialized prompts tailored to the analytical capabilities and information requirements of each AI agent type while maintaining consistency in prompt structure and information presentation across different analytical workflows.

With continued reference to FIG. 4, the prompting process 480 may implement contextual information formatting processes that structure retrieved medical knowledge according to relevance, clinical significance, and hierarchical relationships to support effective language model utilization. The contextual formatting capabilities may analyze knowledge graph information retrieved from the knowledge graphs 440 to identify the most clinically relevant content for specific medical documentation analysis tasks. The formatting processes may organize diagnostic code hierarchies in order of specificity and clinical relevance, structure medication information according to therapeutic classifications and clinical indications, and present laboratory test details with appropriate reference ranges and clinical interpretation guidelines that support accurate analysis and verification operations.

The prompting process 480 may utilize relevance scoring algorithms that assess the clinical significance of retrieved knowledge graph information based on the specific medical entities identified within the original documentation and the analytical objectives of the current processing workflow. The relevance scoring may consider factors such as the specificity of diagnostic codes relative to documented clinical presentations, the therapeutic appropriateness of medication information (such as a prescription) for documented conditions, and the diagnostic utility of laboratory test specifications for clinical validation requirements. The scoring algorithms may enable the prompting process 480 to prioritize the most pertinent knowledge graph information while filtering out excessive or irrelevant details that could overwhelm language model processing or reduce analytical accuracy.

As further shown in FIG. 4, the prompting process 480 may implement multi-source integration capabilities that coordinate the incorporation of information from multiple knowledge graph systems accessed through the computerized systems 420 and computerized systems 430 into comprehensive analytical inputs. The multi-source integration may enable the prompting process 480 to combine diagnostic coding information from ICD-10 Clinical Modification systems with semantic relationship data from SNOMED CT terminologies, pharmaceutical information from medication databases, and laboratory specifications from clinical testing repositories. The integration processes may resolve potential conflicts between different knowledge sources, maintain consistency in information presentation formats, and ensure that combined knowledge provides comprehensive support for medical documentation analysis operations.

The prompting process 480 may utilize sophisticated information synthesis algorithms that merge knowledge from different medical coding systems and knowledge repositories while maintaining the semantic accuracy and clinical utility of the combined information. The synthesis processes may implement cross-referencing operations that identify relationships between concepts from different knowledge systems, hierarchical mapping procedures that align classification structures across different coding frameworks, and semantic reconciliation mechanisms that resolve terminology differences between knowledge sources. The information synthesis capabilities may enable the prompting process 480 to present unified knowledge contexts that leverage the strengths of multiple medical knowledge systems while providing coherent analytical inputs for language model processing.

Referring again to FIG. 4, the prompting process 480 may implement dynamic prompt adaptation mechanisms that modify prompt structure and content based on the characteristics of medical documentation, the complexity of clinical scenarios, and the analytical requirements identified by the AI agent 470. The dynamic adaptation capabilities may enable the prompting process 480 to adjust prompt organization for different medical specialties, modify information presentation formats for various documentation types, and customize analytical instructions based on the specific clinical reasoning requirements of complex medical cases. The adaptive prompt generation may support both routine medical documentation analysis scenarios and sophisticated clinical reasoning tasks that require comprehensive integration of multiple knowledge sources and analytical perspectives.

The prompting process 480 may coordinate with the memory and storage system 402 to implement prompt caching mechanisms that store frequently used prompt templates and knowledge integration patterns to improve processing efficiency during high-volume medical documentation analysis operations. The caching capabilities may maintain optimized prompt structures for common medical entity types, store formatted knowledge graph information for frequently accessed medical codes, and preserve successful prompt patterns that have demonstrated high analytical accuracy in previous processing operations. The prompt caching may reduce processing latency by eliminating redundant prompt generation operations while ensuring that cached prompts remain current with updates to underlying knowledge graph systems and medical coding standards.

As shown in FIG. 4, the prompting process 480 may implement comprehensive prompt validation mechanisms that assess the quality and completeness of generated prompts before submission to language model processing operations provided by the LLM 460 through the computerized systems 420 and computerized systems 430. The prompt validation may include content completeness checks that verify the presence of necessary medical documentation and contextual information, format consistency verification that ensures prompt structure conforms to language model input requirements, and clinical relevance assessment that confirms the appropriateness of included knowledge graph information for the specific analytical objectives. The validation processes may identify potential issues with prompt generation and implement corrective measures to ensure optimal language model performance and analytical accuracy.

The prompting process 480 may utilize sophisticated prompt optimization algorithms that continuously improve prompt generation strategies based on language model performance feedback, analytical accuracy outcomes, and clinical utility assessments provided by healthcare users. The optimization processes may analyze the relationship between prompt structure characteristics and analytical result quality to identify prompt patterns that consistently produce superior medical entity extraction and clinical reasoning outcomes. The prompt optimization may include machine learning algorithms that adapt prompt generation strategies based on accumulated experience with different medical documentation types and clinical scenarios, enabling continuous improvement of prompt effectiveness and analytical performance over time.

With continued reference to FIG. 4, the prompting process 480 may implement specialized prompt generation capabilities for different operational modes including simple mode operations that utilize predetermined prompt templates and complex mode operations that require dynamic prompt formulation based on AI agent-based reasoning processes. The simple mode prompt generation may utilize optimized template structures that efficiently combine medical documentation with predetermined knowledge graph information patterns, while complex mode prompt generation may support sophisticated reasoning workflows that require iterative prompt refinement and adaptive knowledge integration based on intermediate analytical results. The multi-mode prompt generation capabilities may enable the prompting process 480 to support diverse analytical requirements while maintaining optimal processing efficiency for different types of medical documentation analysis scenarios.

The prompting process 480 may coordinate prompt delivery and result collection operations with the communication unit 406 to manage the transmission of augmented prompts to external language model services and the reception of analytical results from the LLM 460 accessed through the computerized systems 420 and computerized systems 430. The prompt delivery coordination may include secure transmission protocols that protect sensitive medical information during communication with external services, result validation mechanisms that verify the completeness and accuracy of received analytical outputs, and error handling procedures that manage communication failures or processing errors that may occur during external language model operations. The coordination capabilities may ensure reliable and secure interaction with external language model services while maintaining data protection standards appropriate for healthcare applications.

As further shown in FIG. 4, the prompting process 480 may implement comprehensive logging and monitoring capabilities that track prompt generation performance, language model processing outcomes, and analytical result quality to support system optimization and quality assurance activities. The logging capabilities may record detailed information about prompt structure characteristics, knowledge graph information utilization, language model processing times, and analytical accuracy metrics to enable systematic analysis of system performance patterns. The monitoring processes may identify trends in prompt effectiveness, detect potential issues with knowledge graph integration, and provide feedback for continuous improvement of prompt generation algorithms and knowledge formatting procedures.

The prompting process 480 may provide configuration management capabilities that enable healthcare organizations to customize prompt generation parameters, knowledge integration preferences, and analytical instruction formats based on institutional requirements, clinical specialty needs, and organizational workflow patterns. The configuration capabilities may include settings for prompt template selection, knowledge graph prioritization criteria, information formatting preferences, and analytical objective specifications that can be tailored to different clinical environments and use case scenarios. The flexible configuration approach may ensure that the prompting process 480 can be effectively adapted to diverse healthcare environments while maintaining consistent analytical capabilities and clinical utility across different implementation scenarios.

Referring again to FIG. 4, the prompting process 480 may implement result interpretation and feedback mechanisms that analyze language model outputs to assess the effectiveness of generated prompts and identify opportunities for prompt optimization and knowledge integration improvement. The result interpretation capabilities may evaluate the accuracy and completeness of medical entity extraction results, assess the clinical relevance of analytical conclusions, and identify patterns in language model performance that correlate with specific prompt characteristics or knowledge integration approaches. The feedback mechanisms may provide systematic input for refining prompt generation algorithms, optimizing knowledge graph utilization strategies, and improving the overall effectiveness of medical documentation analysis operations through continuous learning and adaptation processes.

The prompting process 480 may coordinate with the processors 404 to implement parallel prompt processing capabilities that enable concurrent generation of multiple augmented prompts for batch processing operations or high-volume medical documentation analysis scenarios. The parallel processing coordination may distribute prompt generation tasks across available processing resources, optimize memory utilization during knowledge graph information formatting operations, and coordinate timing of prompt delivery to external language model services to maximize system throughput while maintaining analytical quality. The parallel processing capabilities may enable the prompting process 480 to support large-scale medical documentation analysis operations while maintaining consistent prompt quality and processing efficiency across multiple concurrent analytical workflows.

Referring to FIG. 4, the system 400 may perform comprehensive medical documentation processing operations that coordinate multiple components to receive medical documentation from patient medical files, process the documentation to extract medical entities using large language model capabilities, access biomedical knowledge graphs to obtain contextual information, and generate summaries of medical information that assist physician preparation for patient visits. The system 400 may implement sophisticated workflow coordination mechanisms that manage the sequential execution of documentation retrieval, entity extraction, knowledge graph access, and summary generation operations while maintaining data integrity and processing efficiency throughout the medical information analysis pipeline.

The system 400 may receive medical documentation from patient medical files through the communication unit 406 that establishes secure connections to the medical records 450 maintained by the computerized systems 420 and computerized systems 430. The medical documentation retrieval may access comprehensive patient medical files including electronic health record systems, clinical data repositories, and historical medical documentation that contains relevant clinical information for upcoming patient visits. The documentation retrieval process may utilize standardized healthcare communication protocols to access patient medical files while maintaining appropriate privacy protections and access controls that comply with healthcare regulatory requirements.

As shown in FIG. 4, the system 400 may process the medical documentation to extract medical entities using a large language model provided by the LLM 460 accessed through the computerized systems 420 and computerized systems 430. The medical entity extraction process may utilize the processors 404 to coordinate language model processing operations that identify and classify medical entities within the retrieved patient documentation. The entity extraction may identify diagnoses, medications, laboratory results, and medical procedures documented within patient medical files through sophisticated natural language processing operations that analyze clinical text and generate structured representations of medical information contained within the patient documentation.

The system 400 may access biomedical knowledge graphs to obtain contextual information related to the extracted medical entities through the communication unit 406 that connects to the knowledge graphs 440 maintained by the computerized systems 420 and computerized systems 430. The biomedical knowledge graph access may retrieve structured medical knowledge including diagnostic code hierarchies, medication classifications, laboratory test specifications, and clinical criteria that provide contextual information relevant to the extracted medical entities. The contextual information retrieval may utilize the AI agent 470 to formulate appropriate knowledge graph queries based on the characteristics of extracted medical entities and the clinical context of the patient documentation being analyzed.

With continued reference to FIG. 4, the system 400 may generate a summary of medical information by combining the extracted medical entities with the contextual information from the knowledge graphs through sophisticated information synthesis operations coordinated by the prompting process 480. The summary generation process may integrate the medical entities identified within patient documentation with the structured medical knowledge retrieved from biomedical knowledge graphs to create comprehensive medical information summaries that provide enhanced clinical context and analytical insights. The information combination may organize medical entities according to clinical significance, incorporate relevant diagnostic criteria and clinical guidelines from knowledge graph sources, and structure the combined information into formats that support efficient physician review and clinical decision-making processes.

The medical information summary generation may utilize the memory and storage system 402 to maintain processing state information during complex synthesis operations that combine multiple types of medical entities with diverse contextual information retrieved from different knowledge graph sources. The summary generation process may implement sophisticated data integration algorithms that resolve potential conflicts between different information sources, maintain consistency in medical terminology and coding standards, and ensure that combined information provides accurate and clinically relevant insights for physician preparation activities. The synthesis operations may create structured summaries that highlight relevant medical history, current clinical status, and potential clinical considerations based on the integrated analysis of patient documentation and biomedical knowledge.

As further shown in FIG. 4, the system 400 may provide the summary to assist physician preparation for patient visits through output delivery mechanisms that present the generated medical information summaries in formats suitable for clinical review and utilization. The summary delivery may utilize the communication unit 406 to transmit completed medical information summaries to healthcare providers through secure communication channels that maintain data protection standards appropriate for healthcare applications. The summary presentation may include structured reports that organize medical information according to clinical priority, highlight potential clinical issues or considerations identified through the analysis process, and provide contextual information that supports informed clinical decision-making during patient encounters.

The system 400 may analyze new physician notes in real-time during patient encounters through continuous monitoring capabilities that process medical documentation as documents are created or modified within healthcare information systems. The real-time analysis may utilize streaming data interfaces provided by the communication unit 406 to receive newly created physician notes immediately upon documentation entry, enabling immediate processing and analysis operations that provide timely clinical decision support during active patient care activities. The real-time processing capabilities may coordinate the processors 404, AI agent 470, and prompting process 480 to perform rapid medical entity extraction, knowledge graph access, and summary generation operations within time constraints that support clinical workflow requirements.

Referring again to FIG. 4, the real-time analysis capabilities may enable the system 400 to provide immediate feedback about medical documentation quality, coding accuracy, and clinical consistency while physicians are actively creating patient notes during clinical encounters. The real-time processing may utilize optimized workflow coordination that prioritizes urgent analysis operations, maintains processing state information across multiple concurrent analysis tasks, and coordinates resource allocation to ensure consistent response times during high-volume clinical activity periods. The real-time analysis may support multiple concurrent physician users while maintaining analytical accuracy and system performance through efficient resource management and processing optimization strategies.

The system 400 may provide notifications about potential coding errors or diagnostic inconsistencies identified during the real-time analysis through alert generation mechanisms that evaluate analysis results and create appropriate clinical notifications. The notification generation may utilize the AI agent 470 to assess the clinical significance of identified issues and determine appropriate alert priority levels based on potential impact on patient care and clinical decision-making processes. The notifications may include specific recommendations for addressing identified coding errors, detailed explanations of diagnostic inconsistencies detected through knowledge graph validation, and suggested corrective actions based on clinical guidelines and evidence-based medicine principles retrieved from biomedical knowledge sources.

As shown in FIG. 4, the notification delivery may utilize the communication unit 406 to present alerts through multiple communication channels including visual displays within electronic health record interfaces, mobile notifications to healthcare provider devices, and integrated warnings within clinical documentation systems. The notification system may implement sophisticated alert prioritization algorithms that ensure urgent clinical issues receive immediate attention while preventing alert fatigue through appropriate filtering of less critical findings. The real-time notification capabilities may enable physicians to address potential documentation issues immediately during patient encounters, improving documentation quality and clinical decision support effectiveness through timely intervention and correction opportunities.

The system 400 may support multiple query languages for accessing different types of knowledge graphs through sophisticated database connectivity capabilities implemented by the communication unit 406 and coordinated by the AI agent 470. The multi-language query support may enable the system 400 to access diverse knowledge graph implementations including relational databases that utilize SQL query language, semantic web repositories that require SPARQL query operations, and property graph systems that employ Cypher query syntax. The query language support may provide unified access interfaces that enable seamless integration with different knowledge graph architectures without requiring specialized configuration or manual query formulation for each database type.

With continued reference to FIG. 4, the SQL query support may enable the system 400 to access medical knowledge stored in relational database management systems that organize diagnostic codes, medication databases, and laboratory test classifications in structured table formats. The SQL query capabilities may utilize the processors 404 to generate optimized database queries that retrieve hierarchical code structures, cross-reference medical terminology between different coding systems, and access comprehensive medical knowledge repositories maintained in relational database architectures. The SQL query support may include connection pooling mechanisms that maintain efficient database connections and query optimization strategies that minimize database access latency during real-time medical documentation analysis operations.

The SPARQL query support may enable the system 400 to access medical knowledge represented in Resource Description Framework formats and semantic web technologies that organize medical information as subject-predicate-object triples describing relationships between medical entities. The SPARQL query capabilities may access comprehensive medical ontologies, linked data resources, and semantic knowledge repositories that provide detailed relationship information and conceptual associations between different medical concepts. The SPARQL query support may enable sophisticated semantic reasoning operations that explore complex relationships between medical entities and retrieve contextual information that supports advanced clinical decision support and diagnostic validation processes.

As further shown in FIG. 4, the Cypher query support may enable the system 400 to access medical knowledge stored in property graph databases that represent medical concepts as nodes with associated properties and relationships as edges connecting related medical entities. The Cypher query capabilities may enable complex graph traversal operations that explore multi-hop relationships between medical concepts, diagnostic codes, and clinical procedures through sophisticated relationship path analysis. The Cypher query support may utilize the AI agent 470 to formulate dynamic graph queries that adapt to specific clinical contexts and analytical requirements, enabling comprehensive exploration of medical knowledge networks and semantic relationship structures.

The multiple query language support may enable the system 400 to optimize knowledge graph access based on the specific characteristics and capabilities of different medical knowledge storage systems through intelligent query routing and database selection mechanisms. The query language selection may be determined automatically based on the type of knowledge graph being accessed, the characteristics of medical entities requiring contextual information, and the analytical objectives of the current processing workflow. The unified query interface may abstract the underlying query language differences and provide consistent data access methods to other system components regardless of the specific knowledge graph implementation being utilized, enabling seamless integration of medical knowledge from diverse sources while maintaining processing efficiency and analytical accuracy.

Referring again to FIG. 4, the system 400 may implement comprehensive workflow coordination mechanisms that manage the sequential execution of medical documentation processing operations while maintaining optimal system performance and resource utilization. The workflow coordination may utilize the processors 404 to distribute processing tasks across available computational resources, coordinate timing of knowledge graph access operations to minimize database access conflicts, and manage memory utilization during complex information synthesis operations. The coordinated workflow execution may enable the system 400 to support high-volume medical documentation processing while maintaining consistent analytical quality and response times that meet clinical workflow requirements and physician preparation needs.

The system 400 may provide comprehensive logging and monitoring capabilities that track medical documentation processing performance, knowledge graph access patterns, and summary generation outcomes to support system optimization and quality assurance activities. The monitoring capabilities may utilize the memory and storage system 402 to maintain detailed records of processing operations, analytical accuracy metrics, and physician utilization patterns that enable continuous improvement of system performance and clinical utility. The logging and monitoring may provide feedback for optimizing knowledge graph access strategies, refining summary generation algorithms, and enhancing the overall effectiveness of medical documentation processing operations through systematic analysis of system performance data and clinical outcome measurements.

AI agents and EHR related tasks.

AI agents were mentioned above. Examples and additional information regarding AI agents are provided below.

The AI agents can perform EHR-related tasks by employing KG while maintaining data-provenance and explainability.

The AI agents are imitating human thinking, and in particular the thought processes of physicians.

The computational framework is based on the ReAct paper [Yao et al 2023] and is customized for medical KGs.

### Applications

Each AI agent is tailored-made for a specific task. Tasks can be either real-time (where the physician interacts with the system and gets immediate feedback), batch (data is processed without human involvement). Examples: An AI agent that helps a physician to find the differential diagnosis (real-time). An AI agent that automates medical coding for billing (batch). An AI agent that validates EHR data, looking for potential discrepancies (either real-time or batch). An AI agent that summarizes the patient's EHR records before a visit with a specialist. An AI agent that finds potential matches for clinical trials. A patient-facing customer service AI agent.

Input. The AI agent receives one or more data pieces in various formats. Each datum can be either a structured-data or a free-form, e.g. text (that may originate from OCR), images/videos (e.g. CT or MRI), signals (e.g. EEG, EKG) and others. The structured data will usually include identifiers such as ICD-10-CM codes for diagnoses, LOINC codes for lab tests etc. The text can include terms and verbal descriptions, codes or both. As defined earlier in FIGS. 1-2 we're using an KG-augmented RAG to get the most accurate and specific identifiers. The input can be paths of files in local/remote filesystems, object storage URLs, etc. It can also be ids/references in databases, data warehouses etc.

The AI agent can optionally get a task-description that focuses the AI agent on a specific sub-task, defines the scope, and provides context. E.g. a differential diagnosis AI agent can get the patient's chief complaint, occupation and marital status. A summary AI agent can get the specialty of the medical expert, the reason for visit etc. An AI agent can get a scope that would narrow-down its task. E.g. a temporal scope (e.g. “focus only on records from 2010 onwards”), entity-type based (e.g. “check the consistency of diagnoses only”), geographical (e.g. “restrict your clinical trial search to sites within 20 miles of NYC”), etc. The task-description can be either a free text or some structured format that can be task-specific.

Processing. A computer program produces an output using the following stages: pre-processing, routing, and response.

Pre-processing. The pre-processor fetches the data, and normalizes the input and the task description (if given). Input: E.g. extract text and tables from scanned documents using OCR, recognize named medical entities, and retrieve specific identifiers (as in FIGS. 1-2), use radiology-focused vision models to interpret the imaging, apply time-series models to analyze EKG signals etc.

Task: Convert the textual description (or parts of it) to a structured format. This can be done using various methods depending on the task and complexity. E.g. a rule-based algorithm, pattern-matching, an LLM or any other machine-learning model etc.

The pre-processor may apply various optimizations, e.g.: If a temporal filer is given, the pre-processor may use file metadata to fetch and normalize only files in the given time-range. If a specialty filter or a name of a specific MD was provided, the pre-process will query the DB for the needed progress notes.

Routing. The router decides which components are required to complete the task, and manages the flow of information. If a simple task description was given, the router may use the right tool to provide an answer immediately. E.g. for patient customer service tasks such as “when is my next appointment with Dr. Jekyll?”, the router can directly call the database and with the appropriate query and return an answer. Au contraire, for complex tasks the router may call an LLM-based AI agent and create the proper context for it. I.e. define system and body prompts, tools, choose the most appropriate LLM (e.g. based on accuracy, speed, availability, and costs). See the detailed explanation in the AI agent section. The router handles the information flow with components. It can wait for a final response, or receive partial responses as well. E.g. for progress tracking, time estimation, streaming of LLM responses, etc. Depending on the type of the task and the urgency, the router may act upon partial response without waiting for final results. E.g. if a patient was prescribed a medicine that is dangerous for them.

Response-builder. The response builder receives a structured response from the router, formats it according to the user, the task and the time requirements. E.g. for real-time tasks, it can call an API, store results in a message queue, etc. For batch tasks, it can store the outputs in a pre-defined storage such as a local directory, cloud storage, database etc.

The response-builder may call further post-processing code to act upon the results, e.g. to produce a report of discrepancies, to issue a notification to the user etc.

The response-builder may format its response based on the user. E.g. a physician will receive a response with a professional jargon, while a patient may receive a simplified answer with explanations and references. For patient facing responses, the builder will take care to handle responses which contain medical results with compassion and sensitivity. E.g., if the biopsy result was a malignant tumor, the patient will be referred to their physician.

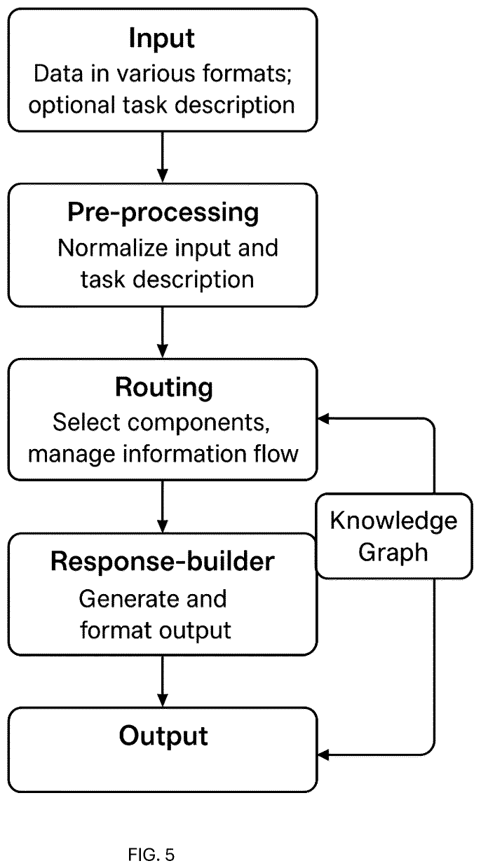

FIG. 5. Illustrates an example of a flow chart of a process related to the AI agent.

### AI Agent

The AI agent receives the normalized inputs, the task description, a list of tools, system prompt and initial body prompt. The AI agent is trying to mimic human reasoning by interleaving observations, reflections and actions. These are task specific, and can be further specialized according to the task description.

### 1. LLM Choices

Here we have several options:
Option A (commercial clinical models): medical-capable models from major providers: Anthropic's Claude for Life Sciences, OpenAI's GPT 5, etc.
Option B:
A fine-tuned/trained model specifically for KG-grounded medical tasks trained using supervised and unsupervised learning on data such as:
   De-identified EHR corpora, e.g. MIMIC-III/IV (notes+structured), eICU
   Task datasets: i2b2/n2c2 clinical NLP datasets Annotated logical forms
   KG: ICD-10, SNOMED CT, LOINC, RxNorm.
   Clinical guidelines & curated knowledge: e.g., UpToDate-like curated rules, public guidelines (e.g., NICE, WHO).
   Human-annotated clinical review logs: clinician corrections and review decisions.

Fine-tune on traces of actions (potentially using bootstrapping) for EHR tasks, using few-shot examples.

### 2. Tools

EHR Connector (FHIR / SQL)

| | |
|---|---|
| • | fetch_patient(patient_id, resources=[“notes”,“labs”,“meds”], |
| | time_window=...) -> {notes:[ ], labs:[ ], ...} |

KG Adapters (one per source: ICD10, SNOMED, LOINC, RxNorm,
Wikidata, hospital KG)

| | |
|---|---|
| • | kg.expand_tree(root_id, depth) -> {nodes:[{id, label, |
| | relations}], provenance_id} |
| • | kg.lookup_term(term) -> {nodes:[{id, label, relations}], |
| | provenance_id |

Retrieval/RAG store (vector DB for note chunks + KG text)

| | |
|---|---|
| • | retrieve(context_query, k=5) -> [documents] |

Verifier Modules (deterministic rules), e.g.

| | |
|---|---|
| | missing tests: |
| | RA → RF, Anti-CCP |
| | Diabetes → HbA1c |
| | CKD → eGFR |
| | timeline consistency: |
| | Diagnosis dated after patient death |
| | Medication start before birth |
| | Chronic disease first noted 1 week ago |
| • | verifier.run(claims, patient_context) -> [{check, status, |
| | severity, evidence}] |

Flow: A ReAct-Like Loop with Typed Tool Calls
a. A dependency DAG
b. LLM proposes a plan.
c. Orchestrator validates plan (typing, security, privacy and legal concerns).
d. Execute first tool action.
e. Feed tool's structured output back to LLM.
f. LLM issues the next step (refine, re-verify, or conclude).
g. Loop ends when LLM emits “FINISH” or max steps reached or verifier returns clean.

### Examples

Task: validate accuracy of diagnoses for this patient.
a. The dependency graph includes (a) labs and measurements, (b) clinical assessments, (c) procedures, and (d) family and social history-all fed to diagnosis.
b. Plan (note some steps can be done in parallel):
   i. Analyze patient's structured data and extract independent variables such as labs, measurements etc. and also the diagnoses (ICD-10-CM codes)
   ii. Analyze patient's notes and extract independent variables such as labs, measurements etc. and also the diagnoses (textual terms).
   iii. For each diagnosis validate it has supporting evidences.
c. Plan validation: make sure that no PHI will be sent to the KGs when calling tools, just terms and identifiers.
d. ReAct Loop trace:
   i. Observation: one of the notes contains the text: “The patient suffers from severe pains in the left knee, likely RA, seropositive.”
   ii. Observation: the note was preprocessed and an ICD-10-CM code was extracted: “Rheumatoid arthritis with rheumatoid factor of left knee without organ or systems involvement: M05.762”
   iii. Thought: I need to convert the ICD code to a SNOMED CUI in order to see the relationships
   iv. Action: call tool icd10 cm_to_snomed_ct (code=′M05.762′)
   V. Observation: found CUI C4759526
   vi. Thought: I need to check the relationships of the SNOMED CT concept
   vii. Action: call tool snomed_ct.expand_node (cui=C4759526)
   viii. Observation: a relation of type ‘interprets’ to ‘Rheumatoid factor measurement’
   ix. Observation: a relation of type ‘Has interpretation’ to ‘Positive’
   x. Thought: to validate this diagnosis, I need to look for RF lab, and make sure there's a positive result
   xi. Action: call tool loinc.lookup_term (“rheumatoid factor”)
   xii. Observation: found LOINC 46437-0
   xiii. Action: call fetch_labs (loinc=‘46437-0’, sort=‘latest’)
   xiv. Observation: 2025 Oct. 7 Negative, 2024 Oct. 7 Negative, 2024 Oct. 7 Negative,
   xv. Thought: No seropositive results
   xvi. Action: call Finish (“Diagnosis Rheumatoid arthritis with rheumatoid factor of left knee without organ or systems involvement not supported by labs”) å

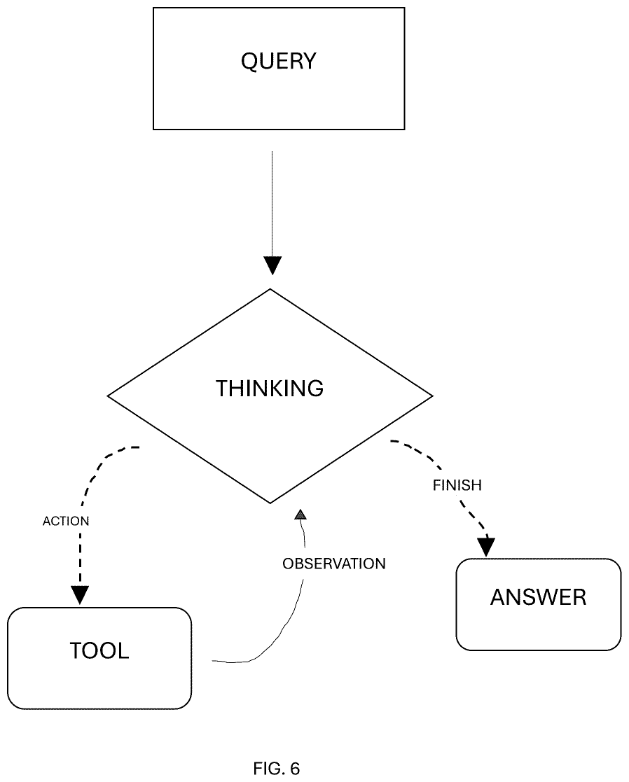

FIG. 6. Illustrates an example of an AI agent ReAct loop.

A number of implementations have been described. Nevertheless, it will be understood that various modifications may be made without departing from the spirit and scope of the disclosure. Accordingly, other implementations are within the scope of the following claims.

Any reference to “may be” may also refer to “may not be”.

In the foregoing detailed description, numerous specific details are set forth to provide a thorough understanding of the one or more embodiments of the disclosure. However, it will be understood by those skilled in the art that the present one or more embodiments of the disclosure may be practiced without these specific details. In other instances, well-known methods, procedures, and components have not been described in detail so as not to obscure the present one or more embodiments of the disclosure.

It will be appreciated that, for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the disclosure may for the most part, be implemented using electronic components and circuits known to those skilled in the art, details will not be explained in any greater extent than that considered necessary as illustrated above, for the understanding and appreciation of the underlying concepts of the present one or more embodiments of the disclosure and in order not to obfuscate or distract from the teachings of the present one or more embodiments of the disclosure.

Any reference in the specification to a method may be applied mutatis mutandis to a system capable of executing the method and may be applied mutatis mutandis to a non-transitory computer readable medium that stores instructions that once executed by a computer result in the execution of the method.

Any reference in the specification to a system and any other component may be applied mutatis mutandis to a method that may be executed by a system and may be applied mutatis mutandis to a non-transitory computer readable medium that stores instructions that may be executed by the system.

Any reference in the specification to a non-transitory computer readable medium may be applied mutatis mutandis to a system capable of executing the instructions stored in the non-transitory computer readable medium and may be applied mutatis mutandis to method that may be executed by a computer that reads the instructions stored in the non-transitory computer readable medium.

Any combination of any module or unit listed in any of the FIGs., any part of the specification and/or any claims may be provided. Especially any combination of any claimed feature may be provided.

In the foregoing specification, the invention has been described with reference to specific examples of embodiments of the invention. It will, however, be evident that various modifications and changes may be made therein without departing from the broader spirit and scope of the invention as set forth in the appended claims.

Those skilled in the art will recognize that the boundaries between logic blocks are merely illustrative and that alternative embodiments may merge logic blocks, circuit elements, or impose an alternate decomposition of functionality upon various logic blocks or circuit elements. Thus, it is to be understood that the architectures depicted herein are merely exemplary, and that in fact many other architectures may be implemented which achieve the same functionality.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Any reference to “consisting”, “having” and/or “including” may be applied mutatis mutandis to “consisting” and/or “consisting essentially of”.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also, for example, in one embodiment, the illustrated examples may be implemented as circuitry located on a single integrated circuit or within a same device. Alternatively, the examples may be implemented as any number of separate integrated circuits or separate devices interconnected with each other in a suitable manner.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

It is appreciated that various features of the embodiments of the disclosure which are, for clarity, described in the contexts of separate embodiments may also be provided in combination in a single embodiment. Conversely, various features of the embodiments of the disclosure which are, for brevity, described in the context of a single embodiment may also be provided separately or in any suitable sub-combination.

It will be appreciated by persons skilled in the art that the embodiments of the disclosure are not limited by what has been particularly shown and described hereinabove. Rather, the scope of the embodiments of the disclosure is defined by the appended claims and equivalents thereof.

1. A computer-implemented method for analyzing medical notes using knowledge graph enhanced language models, comprising:
receiving a medical note containing medical information;
processing the medical note with a large language model to identify medical entities within the medical note;
determining a type of medical entity from the identified medical entities;
accessing a knowledge graph corresponding to the determined type of medical entity, wherein the knowledge graph contains structured medical knowledge relevant to the type of medical entity;
generating an augmented context by retrieving relevant information from the knowledge graph based on the identified medical entities; and
reprocessing the medical note with the large language model using the augmented context to generate enhanced medical analysis results.

2. The computer-implemented method of claim 1, wherein the medical entities comprise diagnoses, medications, laboratory results, or medical procedures.

3. The computer-implemented method of claim 1, wherein the knowledge graph comprises ICD-10 Clinical Modification codes, SNOMED CT codes, or other standardized medical coding systems.

4. The computer-implemented method of claim 1, wherein accessing the knowledge graph operates in a simple mode where an action for accessing the knowledge graph is predetermined based on the type of medical entity identified.

5. The computer-implemented method of claim 4, wherein the simple mode utilizes predefined query templates that are automatically populated with extracted medical entity values.

6. The computer-implemented method of claim 1, wherein accessing the knowledge graph operates in a complex mode where an AI agent-based large language model formulates queries to access the knowledge graph as a tool.

7. The computer-implemented method of claim 6, wherein the AI agent-based large language model utilizes a ReAct framework or LangChain libraries to interact with the knowledge graph.

8. The computer-implemented method of claim 1, wherein the enhanced medical analysis results comprise medical coding verification, diagnosis accuracy checking, or consistency validation.

9. The computer-implemented method of claim 8, further comprising a step of generating alerts for potential inconsistencies between the medical note content and the knowledge graph information.

10. The computer-implemented method of claim 9, further comprising a step of checking for missing laboratory test results based on diagnosis codes and their relationships in the knowledge graph.

11. A system for providing medical information analysis, comprising:
a processor; and
a memory storing instructions that, when executed by the processor, cause the system to:
receive medical documentation from patient medical files;
process the medical documentation to extract medical entities using a large language model;
access biomedical knowledge graphs to obtain contextual information related to the extracted medical entities;
generate a summary of medical information by combining the extracted medical entities with the contextual information from the knowledge graphs; and
provide the summary to assist physician preparation for patient visits.

12. The system of claim 11, wherein the instructions further cause the system to analyze new physician notes in real-time during patient encounters.

13. The system of claim 12, wherein the instructions further cause the system to provide notifications about potential coding errors or diagnostic inconsistencies identified during the real-time analysis.

14. The system of claim 11, wherein the instructions further cause the system to support multiple query languages for accessing different types of knowledge graphs, comprising SQL, SPARQL, and Cypher.

15. The system of claim 11, wherein the instructions further cause the system to perform differential diagnosis analysis by querying knowledge graphs for diseases with similar symptoms or characteristics to extracted medical entities.

16. A computer-readable medium comprising instructions that, when executed by a processor, cause the processor to perform a method for analyzing medical notes using knowledge graph enhanced language models, the method comprising:
receiving a medical note containing medical information;
processing the medical note with a large language model to identify medical entities within the medical note;
determining a type of medical entity from the identified medical entities;
accessing a knowledge graph corresponding to the determined type of medical entity, wherein the knowledge graph contains structured medical knowledge relevant to the type of medical entity;
generating an augmented context by retrieving relevant information from the knowledge graph based on the identified medical entities; and
reprocessing the medical note with the large language model using the augmented context to generate enhanced medical analysis results.

17. The computer-readable medium of claim 16, wherein the medical entities comprise diagnoses, medications, laboratory results, or medical procedures.

18. The computer-readable medium of claim 16, wherein the knowledge graph comprises ICD-10 Clinical Modification codes, SNOMED CT codes, or other standardized medical coding systems.

19. The computer-readable medium of claim 16, wherein accessing the knowledge graph operates in a simple mode where an action for accessing the knowledge graph is predetermined based on the type of medical entity identified.

20. The computer-readable medium of claim 16, wherein the enhanced medical analysis results comprise medical coding verification, diagnosis accuracy checking, or consistency validation.
